(19) United States
(12) Patent Application Publication
(10) Pub. No.: US 2023/0034069 A1
(43) Pub. Date: Feb. 2, 2023

(54) FERMENTATION METHOD AND USES THEREOF

(71) Applicant: CARGILL, INCORPORATED, Wayzata, MN (US)

(72) Inventors: Gary Allan FOLKERT, Bloomington, MN (US); Briana Kristen KOZLOWICZ, Hopkins, MN (US); Chenfeng LU, Edina, MN (US); Thomas William MCMULLIN, Minnetonka, MN (US); Christopher Kenneth MILLER, Andover, MN (US); Kevin WATTS, Minneapolis, MN (US)

(73) Assignee: CARGILL, INCORPORATED, Wayzata, MN (US)

(21) Appl. No.: 17/757,368

(22) PCT Filed: Dec. 18, 2020
(86) PCT No.: PCT/US2020/065883

§ 371 Date: Jun. 15, 2022

Related U.S. Application Data
(60) Provisional application No. 62/952,977, filed on Dec. 23, 2019.

(51) Int. Cl. C12P 7/06; C12N 9/24; C12N 9/16; C12N 15/01

(52) U.S. Cl. CPC C12P 7/06; C12N 9/2402; C12Y 302/01041; C12N 9/16; C12Y 301/03; C12N 15/01

(57) ABSTRACT

Various examples according to the present disclosure provide a fermentation method. The fermentation method includes producing at least about 10 g/L of a bioproduct and one or more heterologous polypeptides by fermenting a medium using an engineered microorganism. About 2 wt % to about 100 wt % of the one or more heterologous polypeptides are encapsulated intercellularly in the engineered microorganism. The method further includes isolating the engineered microorganism including the encapsulated one or more heterologous polypeptides. About 50 wt % to about 100 wt % of the one or more heterologous polypeptides retain functionality following isolation of the engineered microorganism.

### CROSS-REFERENCE TO RELATED APPLICATIONS

This application claims the benefit of U.S. Provisional Application Ser. No. 62/952,977, filed Dec. 23, 2019, and entitled “FERMENTATION METHOD AND USES THEREOF,” which is hereby incorporated by reference herein in its entirety.

### REFERENCE TO A SEQUENCE LISTING SUBMITTED VIA EFS-WEB

The content of the ASCII text file of the sequence listing named “PT740_WO_seglist_ST25.txt” which is 1450 kb in size created on Dec. 17, 2020 and electronically submitted via EFS-Web herewith the application is incorporated by reference in its entirety.

### BACKGROUND

Fermentation processes are used commercially at large scale to produce organic molecules such as ethanol, citric acid and lactic acid. In those processes, a carbohydrate is fed to an organism that is capable of metabolizing it to the desired fermentation product. The carbohydrate and organism are selected together so that the organism is capable of efficiently digesting the carbohydrate to form the product that is desired in good yield. It is becoming more common to use genetically engineered organisms in these processes, in order to optimize yields and process variables, or to enable particular carbohydrates to be metabolized.

### SUMMARY OF THE DISCLOSURE

Various examples according to the present disclosure provide a fermentation method. The fermentation method includes producing at least about 10 g/L of a bioproduct and one or more heterologous polypeptides by fermenting a medium using an engineered microorganism. About 2 wt % to about 100 wt % of the one or more heterologous polypeptides are encapsulated intercellularly in the engineered microorganism. The method further includes isolating the engineered microorganism including the encapsulated one or more heterologous polypeptides. About 50 wt % to about 100 wt % of the one or more heterologous polypeptides retain functionality following isolation of the engineered microorganism.

Various examples according to the present disclosure provide a fermentation method. The fermentation method includes producing at least about 10 g/L ethanol and one or more heterologous polypeptides by fermenting a medium using an engineered yeast including an expression vector, an edited genome, or a combination thereof. About 20 wt % to about 100 wt % of the one or more heterologous polypeptides are encapsulated intercellularly in the engineered yeast. The method further includes isolating the engineered yeast including the encapsulated one or more heterologous polypeptides. One or more heterologous polypeptides exhibit enzymatic activity following isolation of the engineered yeast.

According to various further examples, an engineered microorganism can be adapted to ferment a medium and produce at least about 10 g/L of a bioproduct. The engineered microorganism can include one or more heterologous polypeptides encapsulated in the engineered microorganism. The one or more heterologous polypeptides include at least about 5% of a total cellular polypeptide level in the engineered microorganism. About 50 wt % to about 100 wt % of the one or more heterologous polypeptides retain their functionality following isolation of the engineered microorganism.

According to various further examples, an engineered yeast can include one or more heterologous polypeptides encapsulated in the yeast. The one or more heterologous polypeptides can account for at least about 30% of a total cellular polypeptide level in the yeast. A majority of the one or more heterologous polypeptides can exhibit enzymatic activity following a fermentation process to which the yeast is subjected.

According to various further examples, a mixture can include an engineered microorganism that can be adapted to ferment a medium and produce at least about 10 g/L of a bioproduct. The engineered microorganism can include one or more heterologous polypeptides encapsulated in the engineered microorganism. The one or more heterologous polypeptides include at least about 5% of a total cellular polypeptide level in the engineered microorganism. About 50 wt % to about 100 wt % of the one or more heterologous polypeptides retain their functionality following isolation of the engineered microorganism. The engineered microorganism can be distributed about a substrate.

According to various further examples, a pharmaceutical composition can include a heterologous polypeptide isolated from an engineered microorganism. The engineered microorganism that can be adapted to ferment a medium and produce at least about 10 g/L of a bioproduct. The engineered microorganism can include one or more heterologous polypeptides encapsulated in the engineered microorganism. The one or more heterologous polypeptides include at least about 5% of a total cellular polypeptide level in the engineered microorganism. About 50 wt % to about 100 wt % of the one or more heterologous polypeptides retain their functionality following isolation of the engineered microorganism.

According to various further examples, a fermentation method for producing at least about 10 g/L ethanol and one or more heterologous polypeptides can include fermenting a medium using an engineered yeast comprising an expression vector, an edited genome, or a combination thereof. About 20 wt % to about 100 wt % of the one or more heterologous polypeptides are encapsulated intercellularly in the engineered yeast. The method can further include introducing an amino acid analogue to the engineered microorganism to result in overexpression of the one or more heterologous polypeptides relative to an engineered microorganism that is free of the amino acid analogue. The method can further include isolating the engineered yeast comprising the encapsulated one or more heterologous polypeptides. The one or more heterologous polypeptides can exhibit enzymatic activity following isolation of the engineered yeast. The method can further include isolating the one or more heterologous polypeptides from the isolated engineered yeast. The method can further include distilling a liquid biproduct produced during fermentation to obtain ethanol.

### BRIEF DESCRIPTION OF THE FIGURES

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a flow diagram illustrating a fermentation and processing method, in accordance with various embodiments.

FIG. 2 is a graph showing growth profiles in the presence of varying concentrations of AZC of Example 11, in accordance with various embodiments.

FIG. 3 is a graph showing ethanol production values of Example 23, in accordance with various embodiments.

FIG. 4 is a graph showing glucose consumption profiles of Example 23, in accordance with various embodiments.

FIG. 5 is a graph showing LC/MS analysis of Example 23, in accordance with various embodiments.

FIGS. 6A and 6B are a sequence alignment of the phytase polypeptides of SEQ ID NOs:268, 439, 446, 267, 326, 270, 440, 252, and 311.

FIGS. 7A and 7B are a sequence alignment of the phytase polypeptides of SEQ ID NOs: 268, 439, 446, 267, 326, 270, and 440.

FIG. 8 is a sequence alignment of the phytase polypeptides of SEQ ID NOs:252 and 311. The consensus sequence is SEQ ID NO:448.

FIGS. 9A and 9B are a sequence alignment of the amylase polypeptides of SEQ ID NOs: 373 (amino acids 37-475), 379 (amino acids 25-469), 348 (amino acids 46-477), 393 (amino acids 46-478), 362 (amino acids 144-644), 336 (amino acids 25-511), 356 (amino acids 48-498), and 403 (amino acids 178-703).

FIG. 10 is a sequence alignment of the amylase polypeptides of SEQ ID NOs: 348 and 393. While identities, positives, and gaps were calculated across the full-length sequences, only the first 540 amino acids of the alignment is shown. The consensus sequence is SEQ ID NO:449.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading can occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some examples, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some examples, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some examples, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some examples, the cycloalkyl group can have 3 to about 8-12 ring members, whereas in other examples the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some examples, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain examples there is no hydrocarbyl group.

As used herein, the term “heterologous” refers to a biomolecule such a polypeptide, amino acid, antigen, cofactor, hormone, vitamin, lipid, etc., derived from (e.g., obtained from) a genotypically distinct organism (e.g., a different species) from the rest of the entity to which it is being compared or introduced into.

As used herein, the term “exogenous” refers to a biomolecule originating from outside the host organism.

As used herein, the term “animal” means any organism belonging to the kingdom Animalia and includes, without limitation, poultry, cattle, swine, goat, sheep, cat, dog, mouse, aquaculture, horse, human, or the like.

As used herein “Liquifact”, is corn starch that has undergone liquefaction, with a dextrose equivalents in the range of about 10 to about 15. A corn wet milling process can be used to provide steep-water, which can be used for fermentation. Corn kernels can be steeped and then milled, and separated into their major constituent fractions. Light steep water is a byproduct of the steeping process, and contains a mixture of soluble proteins, amino acids, organic acids, carbohydrates, vitamins, and minerals.

According to various examples of the present disclosure, a fermentation method is described that allows for coproduction of a bioproduct and a heterologous biomolecule such as a heterologous polypeptide. As understood in the art, production of a polypeptide or other biomolecule in an engineered microorganism can be expensive and the time consuming. This makes it potentially undesirable or at least not economical to dedicate the amount of resources necessary to produce these biomolecules absent an additional benefit. However, as discussed in further detail herein, the fermentation methods of the instant disclosure allow for the coproduction of the heterologous biomolecule and a useable (e.g., distillable) amount of a biproduct such as ethanol. The ability to engage in this type of coproduction of a biomolecule and biproduct can make it economically feasible to engage in using the engineered microorganisms described herein to produce the disclosed bioproducts.

In the following discussion where a heterologous polypeptide is used to illustrate various examples of the present disclosure, it is understood that the discussion can pertain to other heterologous biomolecules such as a heterologous amino acid, a heterologous antigen, a heterologous cofactor, a heterologous hormone, a heterologous vitamin, a heterologous lipid, a heterologous pharmaceutical, or a mixture thereof. The heterologous polypeptide that is coproduced with the bioproduct can be substantially encapsulated or retained in an engineered microorganism. The engineered microorganism that includes the heterologous polypeptide can be suited for many acceptable uses. For example, the engineered microorganisms can be integrated into another substance, which can be consumed and the heterologous polypeptide can be delivered to an animal by consuming the substance.

A fermentation process, as described herein, can be anaerobic or aerobic. Under aerobic conditions, engineered microorganisms such as yeast cells can break down sugars to bioproducts such as CO₂ and H₂O. Under anaerobic conditions, engineered microorganism can utilize an alternative pathway to produce bioproducts such as an organic acid (e.g., CO₂, succinic acid, malonic acid) or ethanol. Many of the fermentation procedures described herein are described in the context of an anaerobic fermentation reaction to produce an ethanol bioproduct. Although ethanol is contemplated as the bioproduct, it is possible for other bioproducts to be produced such as a different substituted or unsubstituted (C₁-C₂₀)hydrocarbyl. For example, if a different microorganism is used, fermentation can be used to create methane. Methane fermentation can convert all types of polymeric materials to methane and carbon dioxide under anaerobic conditions. This may be achieved as a result of the consecutive biochemical breakdown of polymers to methane and carbon dioxide in an environment in which a variety of microorganisms including fermentative microbes (acidogens), hydrogen-producing, acetate-forming microbes (acetogens), and methane-producing microbes (methanogens), grow harmoniously and produce the reduced end-products.

Enzymes such as, lipases can convert lipids to long-chain fatty acids. Clostridia and the micrococci are the examples of extracellular lipase producers. Proteins can be generally hydrolyzed to amino acids by proteases, secreted by Bacteroides, Butyrivibrio, Clostridium, Fusobacterium, Selenomonas, and Streptococcus. The amino acids produced can then be degraded to fatty acids such as acetate, propionate, and butyrate, and to ammonia as found in Clostridium, Peptococcus, Selenomonas, Campylobacter, and Bacteroides.

Polysaccharides such as cellulose, starch, and pectin can be hydrolyzed by cellulases, amylases, and pectinases. Most anaerobic bacteria undergo hexose metabolism via the Emden-Meyerhof-Pamas pathway (EMP) which produces pyruvate as an intermediate along with NADH. The pyruvate and NADH thus generated, can then be transformed into fermentation endo-products such as lactate, propionate, acetate, and ethanol by other enzymatic activities which may vary with microorganism species.

Thus, in hydrolysis and acidogenesis, sugars, amino acids, and fatty acids produced by engineered microorganisms by degradation of biopolymers are metabolized to fermentation endo-products such as lactate, propionate, acetate, carbon dioxide, and ethanol by other enzymatic activities which vary with microorganism species. Methanogens such as, Methanosarcina spp. and Methanothrix spp., are also methane producers in anaerobic digestion. Although acetate and H₂/CO₂ are the main substrates available in the natural environment, formate, methanol, methylamines, and CO can also be converted to CH₄.

There are a variety of carbon sources that can be used in the fermentation process of the present disclosure. The raw material for various methods of commercial alcohol production can include a carbohydrate such as a glucose, glucose oligomer, or a mixture thereof. The carbohydrate can be provided from a variety of different sources including corn, wheat, milo, oat, barley, rice, rye, sorghum, potato, whey, sugar beets, taro, cassaya, fruits, fruit juices, and sugar cane. The carbon sources used in the fermentation process can be natural, chemically modified, or genetically modified. The examples of the carbon source that can be fermented by engineered microorganisms of the present disclosure, include corn, canola, alfalfa, rice, rye, sorghum, sunflower, wheat, soybean, tobacco, potato, peanut, cotton, sweet potato, cassaya, coffee, coconut, citrus trees, cocoa, tea, fruits such as, banana, fig, pineapple, guava, mango, oats, barley, vegetables, ornamentals, and conifers. Further examples of carbon sources can include crop plants such as cereals and pulses, maize, wheat, milo, oats, amaranth, rice, sorghum, millet, cassaya, barley, pea, tapioca, taro, potatoes, and other root, tuber, or seed crops. A biomass in the form of wastes from agriculture such as corn stover, rice straw, manure, etc., and biomass crops such as switch grass or poplar trees, and even municipal wastes such as newspaper can all be converted into alcohol. The carbon source can include any appropriate carbon source such as wood, waste paper, manure, cheese whey, molasses, sugar beets or sugar cane. This carbon source can also include unhydrolyzed corn syrup or starch which is an inexpensive carbon source.

As described above, the fermentation methods can use a variety of microorganisms as a suitable host chassis. The microorganisms can be considered to be engineered microorganisms in that they are engineered to produce the heterologous polypeptides described herein. The engineered microorganisms according to the instant disclosure can include eukaryotic organisms or prokaryotic organisms. Examples of prokaryotic organisms include a bacteria. Examples of eukaryotic organisms can include a fungus or yeast. Examples of bacteria that can be used can include Streptococcus, Lactobacillus, Bacillus, Escherichia, Salmonella, Neisseria, Acetobactor, Arthrobacter, Aspergillus, Bifdobacterium, Corynebacterium, Pseudomonas fluorescens, or a mixture thereof. In some specific examples, the engineered microorganism can include Escherichia coli alone. Examples of suitable eukaryotic organisms can include a yeast or another fungi. According to various examples, the yeast can include Saccharomyces cerevisiae, Komagataella sp., Kluyveromyces lactis, Yarrowia lipolytica, Issatchenkia orientalis or a mixture thereof. According to various examples, the fungi can include a filamentous fungi such as Aspergillus, Trichoderma, Myceliophthora thermophila, or a mixture thereof.

The engineered microorganism can be engineered or modified in that it can produce a heterologous polypeptide or other heterologous biomolecule. The engineered microorganism can be a chemically modified or a genetically modified microorganism. According to various embodiments, the cells used in the cell culture are genetically modified by genetic engineering techniques (e.g., recombinant technology), classical microbiological techniques, or a combination of such techniques and can also include naturally occurring genetic variants.

As described in a previous application (WO2016127083, which is incorporated herein by reference in its entirety), naturally secreted enzymes from various fungal and bacterial species generally include a “signal sequence”. In some embodiments, the signal sequence of a naturally secreted polypeptide of interest may need to be removed such that the polypeptide remains inside the engineered microorganism. Various other terms may be used to indicate a “signal sequence” as known in the art, such as where the word “signal” is replaced with “secretion” or “targeting” or “localization” or “transit” or leader,” and the word “sequence” is replaced with “peptide” or “signal.” Generally, a signal sequence is a short amino acid stretch (typically in the range of 5-30 amino acids in length) that is located at the amino terminus of a newly synthesized protein. Most signal peptides include a basic N-terminal region (n-region), a central hydrophobic region (h-region) and a polar C-terminal region (c-region) (e.g., see von Heijne, G. (1986) Nucleic Acids Res. 14, 4683-4690). SignalP (http://www.cbs.dtu.dk/services/SignalP-4.0) can be used to predict whether a polypeptide of interest contains a signal sequence as well as a corresponding cleavage site (SignalP 4.0: discriminating signal peptides from transmembrane regions Thomas Nordahl Petersen, Soren Brunak, Gunnar von Heijne & Henrik Nielsen, Nature Methods, 8:785-786, 2011).

An engineered microorganism, such as a genetically modified microorganism, can include a microorganism in which the genome of the microorganism is edited such that nucleic acid molecules have been inserted, deleted or modified (e.g., mutated; e.g., by insertion, deletion, substitution, and/or inversion of nucleotides), in such a manner that the modifications provide the desired effect production of the heterologous polypeptide and bioproduct during fermentation. As used herein, genetic modifications that result in a decrease in gene expression, in the function of the gene, or in the function of the gene product (e.g., the nutrient such as, protein encoded by the gene) can be referred to as inactivation (complete or partial), deletion, interruption, blockage or down-regulation of a gene. For example, a genetic modification in a gene which results in a decrease in the function of the protein encoded by such gene, can be the result of a complete deletion of the gene (e.g., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity). Genetic modifications which result in an increase in gene expression or function can be referred to as amplification, overproduction, overexpression, activation, enhancement, addition, or up-regulation of a gene. Addition of exogenous polynucleotides to the microorganism to increase gene expression can include maintaining the exogenous polynucleotide(s) on replicating plasmids or integrating the sequence of the exogenous polynucleotide(s) into the genome of the microorganism at a target or localization site. Furthermore, increasing the expression of desired exogenous polynucleotide(s) can include operatively linking the exogenous polynucleotide(s) to native or heterologous transcriptional control elements, for example, a protomer.

The target or localization site for integration of the exogenous polynucleotide sequence may be any suitable site in the host microorganism genome that supports the replication and expression of the desired sequence. The localization site may be, but is not limited to, the FCY1 (SEQ ID NO:450), CAN1 (SEQ ID NO:451), CYB2 (SEQ ID NO:452), GPP1 (SEQ ID NO:453), DLD1 (SEQ ID NO:454), and ADH2 (SEQ ID NO:455).

A microorganism may be modified by methods known in the art and they are within the scope of this disclosure. By way of example only, the method includes manipulating at least one of the structural genes in the nutrients' biosynthetic pathway, optionally manipulating the regulatory controls of the synthetic pathway, and optionally manipulating the nutrients' transport processes out of and into the microorganism. For example, the engineered microorganism may have mutations in a particular gene for biosynthesis. The method can further include manipulating at least one of the structural genes to regulate synthesis of a heterologous polypeptide.

In addition to heterologous polypeptides, the engineered microorganisms can be modified to overproduce a heterologous nutrient such as an essential amino acid, vitamin, hormone, protein, and/or lipid. Where desired, the production of one or more nutrients is under the control of a regulatory sequence that controls directly or indirectly the production in a time-dependent fashion during a fermentation reaction. According to various examples, the regulatory sequences directly or indirectly control the production such that the desired nutrient is produced when the fermentation reaction has reached a desired percentage of completion, such as about 50% completion to about 95% completion, about 60% completion to about 70% completion, at least about 50% of completion, at least about 60% completion, at least about 70%, or at least about 95% completion. When controlled in this manner, the yield of fermentation products such as alcohol and gaseous products is unlikely to be affected.

In some examples, the disclosure includes an engineered microorganism, useful for a fermentation reaction, includes an exogenous polynucleotide encoding a polypeptide, wherein expression of the exogenous polynucleotide may be under the control of a regulatory sequence, e.g. a promoter. In various examples, the regulatory sequences can directly or indirectly suppress expression of an exogenous polynucleotide until the fermentation reaction has reached a desired percentage of completion (e.g., measure by yield of the bioproduct), such as about 50% completion to about 95% completion, about 60% completion to about 70% completion, at least about 50% of completion, at least about 60% completion, at least about 70%, or at least about 95% completion. A variety of suitable regulatory sequences can be employed according to the present disclosure.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

In some embodiments, a microorganism is induced with a construct such as, an expression vector comprising an exogenous sequence encoding a polypeptide of interest. A “construct” is synonymous with “genetic vehicle” used in U.S. 62/952,977. Polynucleotide constructs prepared for introduction into a prokaryotic or eukaryotic host can include a replication system (e.g., a vector) recognized by the host, including the intended polynucleotide fragment encoding the desired polypeptide and can also include transcription and translational initiation regulatory sequences operably linked to the polypeptide-encoding segment. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included where appropriate, from secreted polypeptides of the same or related species, which allow the protein to cross and/or lodge in cell membranes or be secreted from the cell.

A vast number of constructs are suitable for the present disclosure are available. They include both viral and non-viral expression vectors. Non-limiting examples of viral expression vectors are vectors derived from RNA viruses such as retroviruses, and DNA viruses such as adenoviruses and adeno-associated viruses. Non-viral expression vectors include, for example, cosmids, and DNA/liposome complexes. Where desired, the constructs can be engineered to carry regulatory sequences that direct organelle specific expression of the exogenous genes carried therein. For example, leader or signal sequence can be added to direct the exogenous sequence to inclusion bodies of a suitable microorganism. The constructs can be inserted into a host microorganism by any of a number of appropriate means, including electroporation, transfection employing calcium chloride, rubidium chloride, calcium phosphate, DEAE-dextran, or other substances, microprojectile bombardment, lipofection, and infection. The expression vector could be employed for any amino acid or peptide and can be used in the case of E. coli, yeast, or other microorganisms to increase the amino acid or peptide production.

Promoters useful in the practice of the present invention include, but are not limited to constitutive, inducible, temporally-regulated, developmentally regulated, chemically regulated, tissue-preferred, and tissue-specific promoters. The heterologous promoter may be a plant, animal, bacterial, fungal, or synthetic promoter. Suitable promoters for use with the polynucleotide sequences and constructs described herein include, but are not limited to, the TDH3 (gene encoding Glyceraldehyde-3-phosphate dehydrogenase 3), GPD1 (gene encoding glycerol-3-phosphate dehydrogenase 1), URA3 (gene encoding orotidine 5′-phosphate decarboxylase), TEF1 (gene encoding translation elongation factor), ScTDH3 (the TDH3 gene promoter from Saccharomyces cerevisiae), PDC1 (gene encoding pyruvate decarboxylase), ADH1 (gene encoding alcohol dehydrogenase), PGK1 (gene encoding 3-phosphoglycerate kinase), HXK2 (gene encoding hexokinase isoenzyme 2), EFB1 (gene encoding translation elongation factor 1 beta), SAM2 (gene encoding S-adenosylmethionine synthatase), TPS1 (gene encoding trehalase-6-phosphate synthase), CTT1 (gene encoding catylase T), HXK5 (gene encoding hexokinase 5), TPI1 (gene encoding triosephosphate isomerase 1), and GPD2 (gene encoding glycerol-3-phosphate dehydrogenase 1) promoters. Those of skill in the art are familiar with a wide variety of additional promoters for use in various cell types.

In some examples, the engineered microorganism may be free of genetic modification or a vector. However, the engineered microorganism may still overexpress a biomolecule of interest. This can be possible, for example, by introducing an amino acid analog into the engineered microorganism. Introduction of this amino acid analog can result in incorporation of that analog into the biomolecule (e.g., polypeptide). This can lead to a defective polypeptide. To compensate for this, the engineered microorganism can overexpress all or a portion of the polypeptides in the engineered organism. Once the supply of the amino acid analogue is exhausted, functional polypeptides will be produced in an increased proportion, thus resulting in the higher than normal levels of the biomolecule of interest in the engineered microorganism. Although this technique can be used to produce the biomolecules of interest without editing the genome of the engineered microorganism or including a vector therein, it is possible to include the amino acid analogue or analogues in an engineered microorganism that includes an edited genome or an expression vector.

Variants or sequences having substantial identity or homology with the polynucleotides encoding enzymes can be utilized in the practice of the disclosed methods. Such sequences can be referred to as variants or modified sequences. That is, a polynucleotide sequence can be modified yet further to still retain the ability to encode a polypeptide exhibiting the desired activity. Such variants or modified sequences are thus equivalents. Generally, the variant or modified sequence may include greater than about 45%, 45, 50, 55, 60, 65, 70, 75, 80, 85, 90, or 95% sequence identity with the wild-type, naturally occurring or native polynucleotide sequence.

Variants or sequences having substantial identity or homology with the polypeptides described herein can be utilized in the practice of the disclosed methods. Such sequences can be referred to as variants or modified sequences. That is, a polypeptide sequence can be modified yet further to still retain the ability to exhibit the desired activity. Generally, the variant or modified sequence may include or greater than about 45%, 45, 50, 55, 60, 65, 70, 75, 80, 85, 90, or 95% sequence identity with the wild-type, naturally occurring polypeptide sequence.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Some alignment methods take into account conservative amino acid substitutions. Such conservative substitutions, explained in more detail below, generally preserve the charge and hydrophobicity at the site of the amino acid substitution, thus preserving the structure (and therefore function) of the polypeptide. Percent identity for amino acid sequences may be determined as understood in the art. (See, e.g., U.S. Pat. No. 7,396,664). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

In order to optimize the expression of the peptide or protein in the host, the sequence of the peptide or protein can be selected to utilize specific tRNAs that are prevalent in the host. Alternatively, selected tRNAs may be co-expressed in the host to facilitate expression of the peptide or protein. Alternatively, single and multiple codon usage patterns can be adjusted for optimal yield, folding, and localization. The recombinantly-engineered peptide or proteins can include specific sequences to facilitate purification of the peptide or proteins. The proteins may also include “leader sequences” that target the protein to specific locations in the host cell such as the periplasm, or to target the protein for secretion. The recombinantly-engineered peptide or proteins can also include protease cleavage sites to facilitate cleavage of the proteins in the abomasum and enhance delivery of amino acids in the peptide or protein to the small intestine. For example, one such protease is pepsin, one of the protein-digesting enzymes of the abomasum in cattle. Pepsin demonstrates a preferential cleavage of peptides at hydrophobic preferentially aromatic, residues in the P1 and P1′ positions. In particular, pepsin cleaves proteins on the carboxy side of phenylalanine, tryptophan, tyrosine, and leucine. More favorably, the polypeptide is readily cleavable by animal proteases generally.

In some examples of the disclosure, a microorganism can be engineered in such a way that the modified microorganism is enriched in vitamins. The vitamins include but are not limited to, vitamin A (retinol), vitamin B1 (thiamine), vitamin B2 (riboflavin), vitamin B3 (Niacin), vitamin B5 (Pantothenic acid), vitamin B6 (Pyridoxine), vitamin B7 (Biotin), vitamin B9 (Folic acid), vitamin B12 (cyanocobalamin), vitamin C (ascorbic acid), vitamin D1-D4 (lamisterol, ergocalciferol, calciferol, dihydrotachysterol, 7-dehydrositosterol), vitamin E (tocopherol), and vitamin K (naphthoquinone).

The engineered microorganism as discussed above can be cultured in a fermentation medium for production of nutrients, for example, the bioproduct and the heterologous polypeptide. An appropriate, or effective, fermentation medium refers to any medium in which a modified microorganism of the present invention, when cultured, is capable of producing nutrients. Such a medium can be an aqueous medium comprising assimilable carbon, nitrogen and phosphate sources. Such a medium can also include appropriate salts, minerals, metals, and other nutrients. It should be recognized, however, that a variety of fermentation conditions are suitable and can be selected by those skilled in the art.

As used herein, “fermentation conditions” refers to the collective environment of the microorganism during fermentation and includes the temperature, oxygen content, humidity, medium composition and other characteristics necessary to product the bioproduct and heterologous polypeptides described herein. While fermentation conditions generally applicable to the fermentation embodiments described herein are recited, a skilled artisan would recognize other fermentation conditions suitable for use with the engineered microorganisms and methods described herein.

Sources of assimilable carbon which can be used in a suitable fermentation medium include, but are not limited to, sugars and their polymers, including, dextrin, sucrose, maltose, lactose, glucose, fructose, mannose, sorbose, arabinose and xylose; fatty acids; organic acids such as acetate; primary alcohols such as ethanol and n-propanol; and polyalcohols such as glycerine. Examples of carbon sources in the present disclosure include monosaccharides, disaccharides, and trisaccharides.

The concentration of a carbon source, such as glucose, in the fermentation medium should promote cell growth, but not be so high as to repress growth of the microorganism used. Typically, fermentations are run with a carbon source, such as glucose, being added at levels to achieve the desired level of growth and biomass. In other embodiments, the concentration of a carbon source, such as glucose, in the fermentation medium is greater than about 1 g/L, greater than about 2 g/L, or greater than about 5 g/L. In addition, the concentration of a carbon source, such as glucose, in the fermentation medium can be less than about 100 g/L, less than about 50 g/L, or less than about 20 g/L. It should be noted that references to fermentation component concentrations can refer to both initial and/or ongoing component concentrations. In some examples, it may be desirable to allow the fermentation medium to become depleted of a carbon source during fermentation.

Sources of assimilable nitrogen that can be used in a suitable fermentation medium include, but are not limited to, simple nitrogen sources, organic nitrogen sources, and complex nitrogen sources. Such nitrogen sources include anhydrous ammonia, ammonium salts, and substances of animal, vegetable, and/or microbial origin. Suitable nitrogen sources include, but are not limited to, protein hydrolysates, microbial biomass hydrolysates, peptone, yeast extract, ammonium sulfate, urea, and amino acids. Hydrolyzed grain products form a suitable nitrogen source. In various examples, the concentration of the nitrogen sources, in the fermentation medium can be greater than about 0.1 g/L, greater than about 0.25 g/L, or greater than about 1.0 g/L. Beyond certain concentrations, however, the addition of a nitrogen source to the fermentation medium may not be advantageous for the growth of the microorganisms. As a result, the concentration of the nitrogen sources, in the fermentation medium may be less than about 20 g/L, less than about 10 g/L or less than about 5 g/L. Further, in some instances it may be desirable to allow the fermentation medium to become depleted of the nitrogen sources during fermentation.

The effective fermentation medium can contain other compounds such as inorganic salts, vitamins, trace metals, or growth promoters. Such other compounds can also be present in carbon, nitrogen or mineral sources in the effective medium or can be added specifically to the medium.

The fermentation medium can also contain a suitable phosphate source. Such phosphate sources include both inorganic and organic phosphate sources. Suitable phosphate sources include, but are not limited to, phosphate salts such as mono or dibasic sodium and potassium phosphates, ammonium phosphate and mixtures thereof. Typically, the concentration of phosphate in the fermentation medium can be greater than about 1.0 g/L, about 2.0 g/L, or about 5.0 g/L. Beyond certain concentrations, however, the addition of phosphate to the fermentation medium may not be advantageous for the growth of the microorganisms. Accordingly, the concentration of phosphate in the fermentation medium can be less than about 20 g/L, less than about 15 g/L, or less than about 10 g/L.

A suitable fermentation medium can also include a source of magnesium, which may be in the form of a physiologically acceptable salt, such as magnesium sulfate heptahydrate, although other magnesium sources in concentrations that contribute similar amounts of magnesium can be used. The concentration of magnesium in the fermentation medium can greater than about 0.5 g/L, about 1.0 g/L, or about 2.0 g/L. Beyond certain concentrations, however, the addition of magnesium to the fermentation medium may not be advantageous for the growth of the microorganisms. Accordingly, the concentration of magnesium in the fermentation medium can be less than about 10 g/L, less than about 5 g/L, or less than about 3 g/L. Further, in some instances it may be desirable to allow the fermentation medium to become depleted of a magnesium source during fermentation.

The fermentation medium can also include a biologically acceptable chelating agent, such as the dihydrate of trisodium citrate. In such instance, the concentration of a chelating agent in the fermentation can be greater than about 0.2 g/L, greater than about 0.5 g/L, or greater than about 1 g/L. Beyond certain concentrations, however, the addition of a chelating agent to the fermentation medium may not be advantageous for the growth of the microorganisms. Accordingly, the concentration of a chelating agent in the fermentation medium can be less than about 10 g/L, less than about 5 g/L, or less than about 2 g/L.

The fermentation medium can also initially include a biologically acceptable acid or base to maintain the desired pH of the fermentation medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof.

The fermentation medium can also include a biologically acceptable calcium source, including, but not limited to, calcium chloride. Typically, the concentration of the calcium source, such as calcium chloride, dihydrate, in the fermentation medium is within the range of from about 5 mg/L to about 2000 mg/L, about 20 mg/L to about 1000 mg/L, or about 50 mg/L to about 500 mg/L.

The fermentation medium can also include sodium chloride. Typically, the concentration of sodium chloride in the fermentation medium can be within the range of from about 0.1 g/L to about 5 g/L, about 1 g/L to about 4 g/L, or from about 2 g/L to about 4 g/L.

The fermentation medium can also include trace metals. Such trace metals can be added to the fermentation medium as a stock solution that, for convenience, can be prepared separately from the rest of the fermentation medium. Typically, the amount of such a trace metals solution added to the fermentation medium can be greater than about 1 mil/L, greater than about 5 ml/L, or greater than about 10 ml/L. Beyond certain concentrations, however, the addition of a trace metals to the fermentation medium may not be advantageous for the growth of the microorganisms. Accordingly, the amount of such a trace metals solution added to the fermentation medium can be less than about 100 ml/L, less than about 50 ml/L, or less than about 30 ml/L. A suitable trace metal solution can include, but is not limited to sodium selenate; ferrous sulfate; heptahydrate; cupric sulfate, pentahydrate; zinc sulfate, heptahydrate; sodium molybdate, dihydrate; cobaltous chloride; Selenium or chromium solution; hexahydrate; and manganous sulfate monohydrate. Hydrochloric acid may be added to the stock solution to keep the trace metal salts in solution.

The fermentation medium can also include vitamins. Such vitamins can be added to the fermentation medium as a stock solution that, for convenience, can be prepared separately from the rest of the fermentation medium, Typically, the amount of such vitamin solution added to the fermentation medium can be greater than 1 ml/L, greater than 5 mL or greater than 10 ml/L. Beyond certain concentrations, however, the addition of vitamins to the fermentation medium may not be advantageous for the growth of the microorganisms. Accordingly, the amount of such a vitamin solution added to the fermentation medium can be less than about 50 ml/L, less than 30 ml/L, or less than 20 ml/L.

The fermentation medium can also include sterols. Such sterols can be added to the fermentation medium as a stock solution that is prepared separately from the rest of the fermentation medium. Sterol stock solutions can be prepared using a detergent to aid in solubilization of the sterol. Typically, an amount of sterol stock solution is added to the fermentation medium such that the final concentration of the sterol in the fermentation medium is within the range of from about 1 mg/L to 3000 mg/L, about 2 mg/L to 2000 mg/L, or about 5 mg/L to 2000 mg/L.

Engineered microorganisms of the present disclosure can be cultured in conventional fermentation modes, which include, but are not limited to, batch, fed-batch, cell recycle, and continuous. In a fed-batch mode, when during fermentation some of the components of the medium are depleted, it may be possible to initiate the fermentation with relatively high concentrations of such components so that growth is supported for a period of time before additions are required. The ranges of these components are maintained throughout the fermentation by making additions as levels are depleted by fermentation. Levels of components in the fermentation medium can be monitored by, for example, sampling the fermentation medium periodically and assaying for concentrations. Alternatively, once a standard fermentation procedure is developed, additions can be made at timed intervals corresponding to known levels at particular times throughout the fermentation. The additions to the fermenter may be made under the control of a computer in response to fermenter conditions or by a preprogrammed schedule. Moreover, to avoid introduction of foreign microorganisms into the fermentation medium, addition is performed using aseptic addition methods, as are known in the art. In addition, a small amount of anti-foaming agent may be added during the fermentation, or anti-foaming device may be employed.

The temperature of the fermentation medium can be any temperature suitable for growth and production of the nutrients of the present disclosure. For example, prior to inoculation of the fermentation medium with an inoculum, the fermentation medium can be brought to and maintained at a temperature in the range of from 0° C. to 45° C., 0° C. to 32° C., 0° C. to 20° C., 20° C. to 45° C., 25° C. to 40° C., or 28° C. to 32° C.

The pH of the fermentation medium can be controlled by the addition of acid or base to the fermentation medium. In such cases when ammonia is used to control pH, it also conveniently serves as a nitrogen source in the fermentation medium. The pH can be maintained from about 3.0 to about 8.0, about 3.5 to about 7.0, or from about 4.0 to about 6.5.

The fermentation medium can also be maintained to have a dissolved oxygen content during the course of fermentation to maintain cell growth and to maintain cell metabolism for production of the nutrients. The oxygen concentration of the fermentation medium can be monitored using known methods, such as through the use of an oxygen electrode. Oxygen can be added to the fermentation medium using methods such as agitation and aeration of the medium by stirring, shaking or sparging. The oxygen concentration in an aerobic fermentation medium can be in the range of from about 20% to about 100% of the saturation value of oxygen in the medium based upon the solubility of oxygen in the fermentation medium at atmospheric pressure and at a temperature in the range of from about 20° C. to about 40° C. Periodic drops in the oxygen concentration below this range may occur during fermentation, however, without adversely affecting the fermentation.

Although aeration of the medium has been described herein in relation to the use of air, other sources of oxygen can be used. Particularly useful is the use of an aerating gas that contains a volume fraction of oxygen greater than the volume fraction of oxygen in ambient air. In addition, such aerating gases can include other gases which do not negatively affect the fermentation. In some embodiments, fermentation is performed under conditions well established in the art.

The fermentation medium can be inoculated with an actively growing culture of microorganisms of the present disclosure in an amount sufficient to produce, after a reasonable growth period, a high cell density. Suitable inoculation cell densities are within the range of from about 0.01 g/L to about 10 g/L, about 0.2 g/L to about 5 g/L, or from about 0.05 g/L to about 1.0 g/L, based on the dry weight of the cells. In production scale fermenters, however, greater inoculum cell densities are preferred. The cells are then grown to a cell density in the range of from about 10 g/L to about 100 g/L, about 20 g/L to about 80 g/L, or about 50 g/L to about 70 g/L. The residence times for the engineered microorganisms to reach the desired cell densities during fermentation can be less than about 200 hours, less than about 120 hours, or less than about 96 hours.

The engineered microorganisms described herein can be designed to produce a desired amount of the bioproducts described herein. For example the engineered microorganisms can produce at least about 10 g/L of bioproduct during fermentation, about 20 g/L, about 30 g/L, about 40 g/L, about 50 g/L, about 60 g/L, about 70 g/L, about 80 g/L, about 90 g/L, about 100 g/L, about 110 g/L, about 120 g/L, about 130 g/L, about 140 g/L, about 150 g/L, about 160 g/L, about 170 g/L, about 180 g/L, about 190 g/L, about 200 g/L, about 10 g/L to about 200 g/L, about 40 g/L to about 160 g/L, or about 50 g/L to about 100 g/L. The amount of bioproduct produced is chosen to be a useable or threshold amount of bioproduct. For example, if the bioproduct includes ethanol, a useable amount of ethanol can be considered to be an amount of ethanol that is capable of being distilled.

In examples where the engineered microorganism comprises the one or more encapsulated heterologous polypeptides, following fermentation, those one or more heterologous polypeptides can constitute at least about 10% of a total cellular polypeptide level in the engineered microorganism, at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, or at least about 90%, or are in a range of from about 10% to about 70% of a total cellular polypeptide level in the engineered microorganism, or about 15% to about 30% of a total cellular polypeptide level in the engineered microorganism.

Notably, a high degree of the heterologous polypeptides produced during fermentation using the engineered microorganisms are able to retain their functionality following fermentation and isolation of the engineered microorganism. For example, if one or more of the heterologous polypeptides are enzymes, then the enzymes are able to substantially retain catalytic activity following fermentation. If one or more of the heterologous polypeptides are non-enzymatic, then retaining their functionality can include retaining their structure (e.g. their tertiary structure). According to various examples about 70 wt % to about 100 wt % of the of the one or more heterologous polypeptides retain functionality following isolation of the engineered microorganism, about 85 wt % to about 90 wt %, less than, equal to, or greater than about 70 wt %, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 wt %.

The heterologous polypeptides produced by the fermentation methods described herein can be understood to constitute different classes of heterologous polypeptides. For example, the fermentation method can produce a first class of heterologous polypeptide and a second class of heterologous polypeptides. According to some examples, a first class of the heterologous polypeptides can be enzymatic while another class of heterologous polypeptides can be non-enzymatic. Examples of enzymatic heterologous polypeptides can include one or more enzymes chosen from oxidoreductases, transferases, hydrolases, lyases, isomerases, ligases, and translocases. Oxioreductases, designated as enzyme class EC1, catalyze redox reactions. Transferases, designated as enzyme class EC2, catalyze the transfer or exchange of functional groups or motifs amongst a substrate. Hydrolases, designated as enzyme class EC3, accelerate the hydrolysis of substrates. Lyases, designated as enzyme class EC4, promote the removal of a group from a substrate to catalyze a double bond reaction or catalyze the reverse of the reaction. Isomerases, designated as enzyme class EC5, facilitate the conversion of isomers, geometric isomers or optical isomers. Ligases, designated as enzyme class EC6, catalyze the synthesis of two molecular substrates into one molecular compound with the release energy. Translocases, designated as enzyme class EC6, catalyze the movement of ions or molecules across membranes or their separation within membranes. Non-limiting examples of heterologous polypeptides of the various classes include a phytase, an alpha-amylase, a glucoamylase, a glucanase, a glycosidase a lipase, an alkaline extracellular protease, an invertase, a galactanase I, an α-amylase, an α-galactosidase, a peroxidase, an aspartic proteinase II, or a mixture thereof. Examples of non-enzymatic heterologous polypeptide can include an al-antitrypsin, a hiruidin, a transferrin, an insulin, a serum albumin, collagen, an interferon-alpha 2b, gluten, collagen, gelatin, elastins, or mixtures thereof. Some heterologous polypeptides can have pharmaceutical properties (e.g., small molecules or proteins with pharmaceutical properties). Other heterologous polypeptides can be used for personal care such as a heterologous hydrolysate (a polypeptide that that can boost the activity of hyaluronic acid).

The heterologous polypeptides produced by the fermentation methods described herein may be one or more phytase enzymes. Phytase enzymes (EC 3.1.3.8, 3.1.3.26, and 3.1.3.72) catalyze the hydrolysis of phytic acid to release inorganic phosphorous. Examples of phytase polypeptides suitable for use in the methods described herein include, but are not limited to, the phytase enzymes from Rhizomucor pusillus, Arabidopsis thaliana, Saccharomyces mikatae, A. fumigatus, Thermomyces lanuginosus, A. niger, Peniophora lycii, Agrocybe pediades, Ceriporia sp., Trametes pubescens, Bacillus licheniformis, Bacillus sp. DS11, Aspergillus awamori, Aspergillus flavus, B. amyloliquefaciens, Pectobacterium carotovorum, Lupinus albus, Pediococcus pentosaceus, Pediococcus claussenii, Magnaportha grisea, Lactobacillus rhamnosus GG, Lactobacillus casei, Lactococcus lactis, Lactobacillus amylovorus, Rhizopus microspores, Mus musculus, Citrobacter freundii, Citrobacter werkmanii, Kosakonia sacchari, Citrobacter amalonaticus, Cronobacter sakazakii, Cronobacter turicensis, Azorhizobium caulinodans ORS 571, Xanthomonas campestris pv. raphani 756C, Azospirillum sp. B510, Escherichia coli, Escherichia albertii, Candidatus hamiltonella defense, Edwardsiella ictaluri 93-146, Myroides odoratus, Advenella kashmirensis, Penicillium oxalicum, Cladosporium, Parcubacteria group bacterium, Streptomyces fradiae, Natranaerobius thermophilus, Marinomonas posidonica, Leptotrichia buccalis, Rhodothermus marinus, Sphaerobacter thermophilus, Thermotoga petrophila, Paenibacillus sp., Microbulbifer aggregans, Dethiobacter alkaliphilus, Salinispora tropica, Arenibacter algicola, Proteiniborus sp., Marinimicrobia bacterium, Halorhabdus tiamatea, Streptomyces pratensis, Geobacter uraniireducens, Streptomyces fulvissimus, Rubellimicrobium mesophilum, Akkermansia muciniphila, Beggiatoa sp., Pseudomonas fluorescens, Aspergillus oryzae, Talaromyces thermophilus, Aspergillus japonicus, Streptomyces sp., Thielavia heterothallica, Pseudomonas mendocina, Cellvibrio sp., Asticcacaulis biprosthecum, Marinomonas gallaica, Marinomonas mediterranea, Thermobaculum terrenum, Moraxella catarrhalis, Erythrobacter gangjinensis, Novosphingobium sp., Lilium longiflorum, Bacillus mycoides, and Citrobacter braakii. The phytase enzyme may include a sequence at least 50%, 55%, 60%, 65%, 70%, 75%, 80%, 85%, 90%, 95%, 98%, or 99% identical to any one of SEQ ID NOs:240-326 and 437-447. The phytase enzyme may include a sequence at least 50%, 55%, 60%, 65%, 70%, 75%, 80%, 85%, 90%, 95%, 98%, or 99% identical to any one of SEQ ID NOs:273, 267, 244, 252, 268, 269, 246, 270, 326, 271, 311, and 437-447. The phytase enzyme may include a sequence at least 50%, 55%, 60%, 65%, 70%, 75%, 80%, 85%, 90%, 95%, 98%, or 99% identical to any one of SEQ ID NOs: 267, 252, 268, 270, 326, 311, 439, 440, and 446. The phytase enzyme may include a sequence at least 50%, 55%, 60%, 65%, 70%, 75%, 80%, 85%, 90%, 95%, 98%, or 99% identical to any one of SEQ ID NOs: 267, 269, 268, 270, 326, 439, 440, and 446. The phytase enzyme may include a sequence at least 50%, 55%, 60%, 65%, 70%, 75%, 80%, 85%, 90%, 95%, 98%, or 99% identical to any one of SEQ ID NOs: 252 and 311.

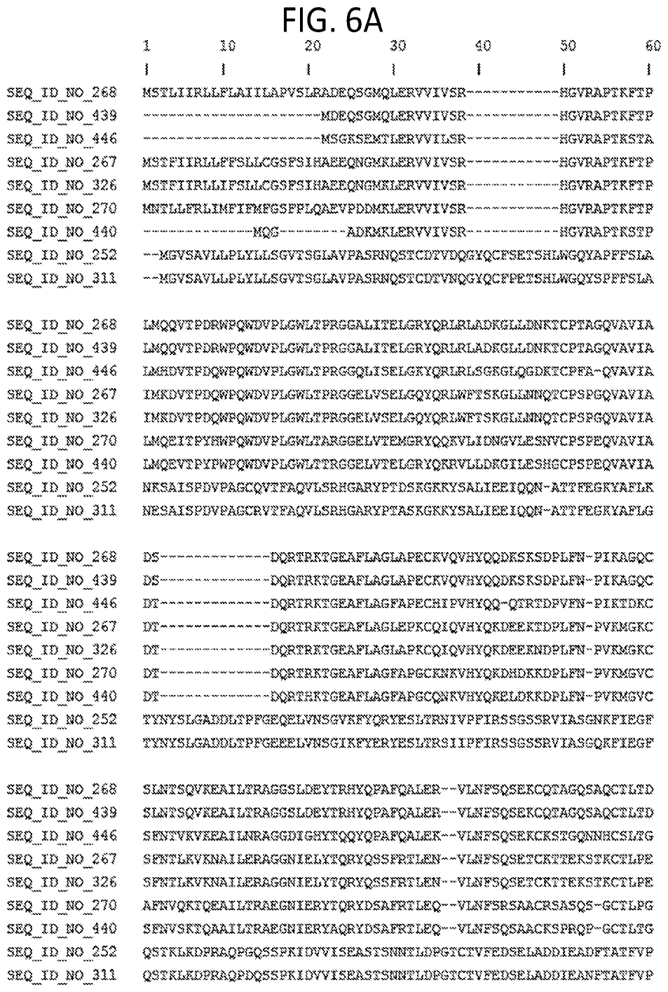

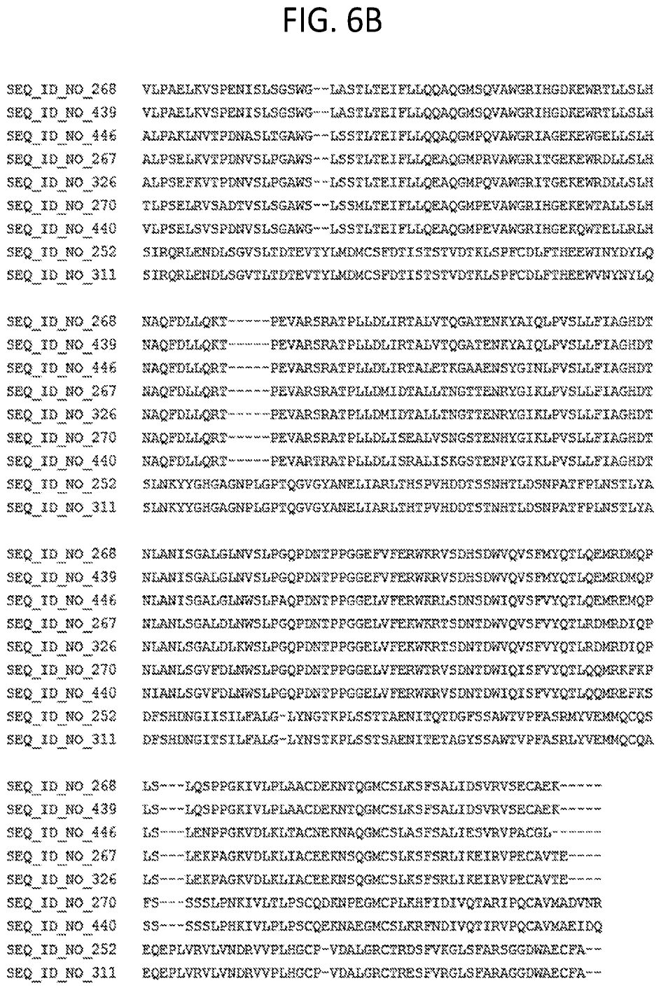

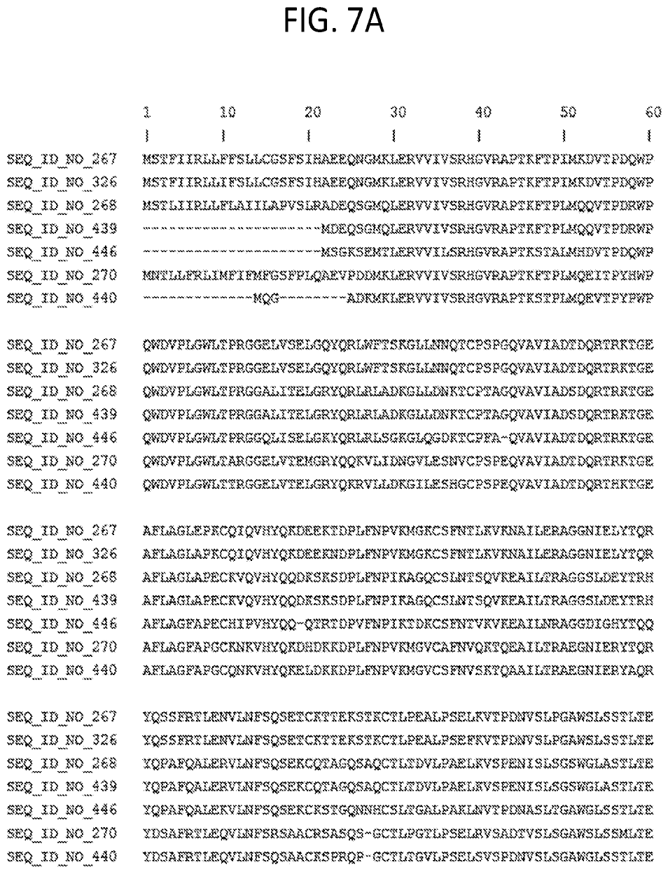

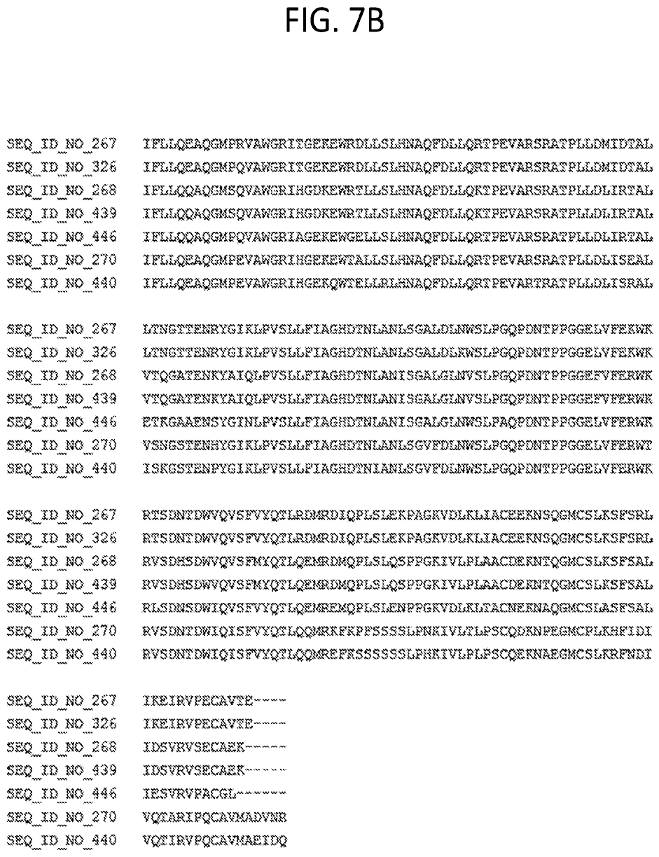

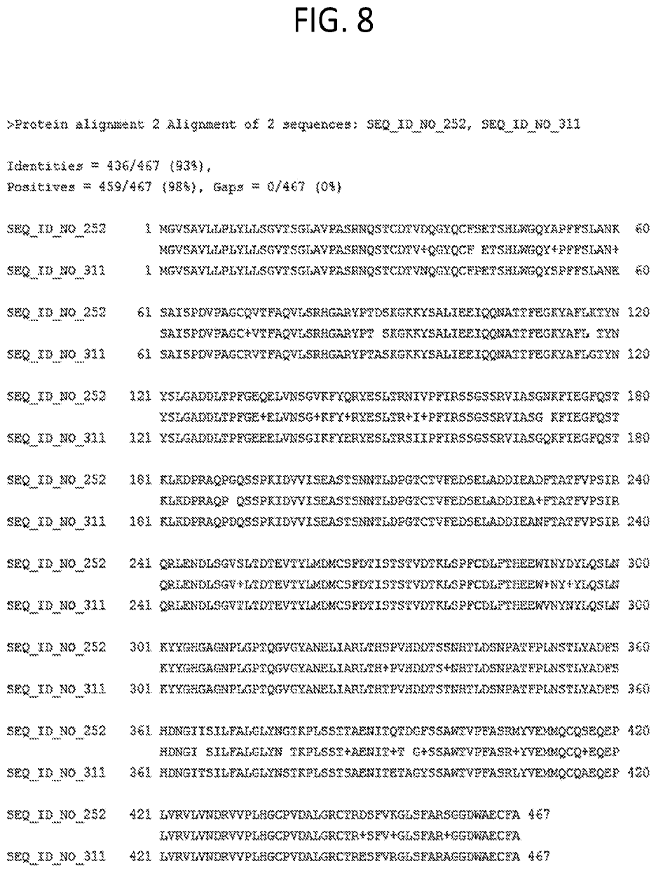

FIGS. 6, 7, and 8 show sequence alignments including some of the phytase polypeptides disclosed as SEQ ID NOs: 240-326 and 437-447. Based on these alignments it becomes immediately apparent to a person of ordinary skill in the art that various amino acid residues may be altered (i.e. substituted, deleted, etc.) without substantially affecting the phytase activity of the polypeptide. For example, a person of ordinary skill in the art would appreciate that substitutions in a reference phytase polypeptide could be based on alternative amino acid residues that occur at the corresponding position in other phytase polypeptides from other species. SEQ ID NOs: 240-326 and 437-447 may also include phytase polypeptides that are not shown in FIGS. 6, 7, and 8. A person of ordinary skill in the art, however, could easily align these polypeptide sequences with the polypeptide sequences shown in FIGS. 6, 7, and 8 to determine what additional variants could be made to the phytase polypeptides.

The heterologous polypeptides produced by the fermentation methods described herein may be one or more amylase enzymes, for example, an alpha amylase. Alpha amylase enzymes (EC 3.2.1.1) hydrolyze alpha bonds of large alpha-linked polysaccharides, such as starch and glycogen, to yield smaller monosaccharides, disaccharides, or polysaccharides, such as glucose, maltose, or dextrin. Examples of amylase polypeptides suitable for use in the methods described herein include, but are not limited to, the amylase enzymes from Lachnospiraceae bacterium G11, Butyrivibrio fibrisolvens, Blautia schinkii, Lachnospiraceae bacterium, Schwanniomyces occidentalis, Parabacteriodes goldsteinii, Bacteroides vuglatus, Bacteroides thetaiotamicron, Bacteroides xylanisolvens XB1A, Drosophila melanogaster, Saccharomycopsis fibuligera, Haloarcula japonica, Bacillus lichenmformis, Bacillus amyloliquefaciens, Aspergillus oryzae, Aspergillus fumigatus, Aspergillus awamori, Apis mellfera, Acarus siro, Aspergillus niger, Aspergillus rambellii, Grosmannia clavigera kw1407, Penicillium nordicum, Penicillium freii, Aspergillus sydowii, Aspergillus kawachii, Alteromonas macleodii, Streptococcus sp., Dictyoglomus thermophilum, Xanthomonas campestris, Streptomyces lividans TK24, Crassostrea gigas, Hordeum vulgare, Streptomyces thermoviolaceus, Geobacillus thermodenitrificans, Lactobacillus amylovorus, Bacillus sp., Pseudomonas sp. KFCC10818, Tribolium castaneum, Sulfolobus solfataricus, Thermoactinomyces vulgaris, Thermococcus sp., Thermotoga maritima, Streptomyces lividans, Bacillus subtilis, Lactobacillus manihotivorans, Bacillus halodurans, Drosophila yakuba, Streptomyces lividans, Clostridium acetobutylicum, Penicillium oxalicum, Zea mays, Mus musculus, Aedes aegypti, Thermomyces langinosus, Escherichia coli, Lipomyces spencermartinsiae, and Cellosobruchus chinensis. The amylase polypeptide may include a sequence at least 50%, 55%, 60%, 65%, 70%, 75%, 80%, 85%, 90%, 95%, 98%, or 99% identical to any one of SEQ ID NOs:327-414. The amylase polypeptide may include a sequence at least 50%, 55%, 60%, 65%, 70%, 75%, 80%, 85%, 90%, 95%, 98%, or 99% identical to any one of SEQ ID NOs:379, 375, 336, 373, 393, 378, 362, 348, 356, and 403. The amylase polypeptide may include a sequence at least 50%, 55%, 60%, 65%, 70%, 75%, 80%, 85%, 90%, 95%, 98%, or 99% identical to any one of SEQ ID NOs:373, 379, 348, 393, 362, 336, 356, and 403. The amylase polypeptide may include a sequence at least 50%, 55%, 60%, 65%, 70%, 75%, 80%, 85%, 90%, 95%, 98%, or 99% identical to any one of SEQ ID NOs:375 and 378. The amylase polypeptide may include a sequence at least 50%, 55%, 60%, 65%, 70%, 75%, 80%, 85%, 90%, 95%, 98%, or 99% identical to any one of SEQ ID NOs:348 and 393. The amylase polypeptide may include a sequence at least 50%, 55%, 60%, 65%, 70%, 75%, 80%, 85%, 90%, 95%, 98%, or 99% identical to any one of SEQ ID NOs:379 and 373.

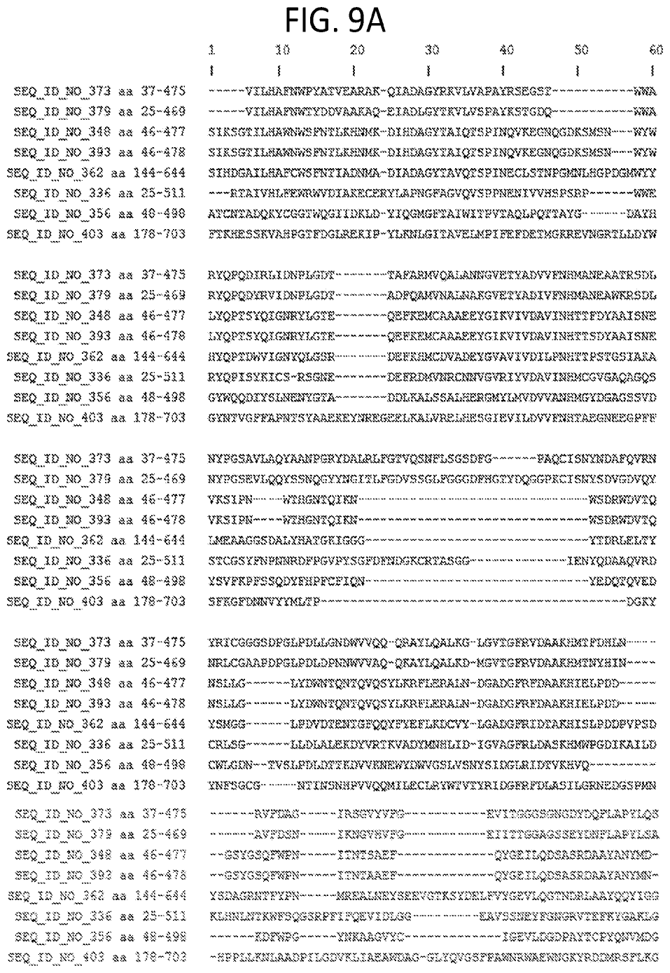

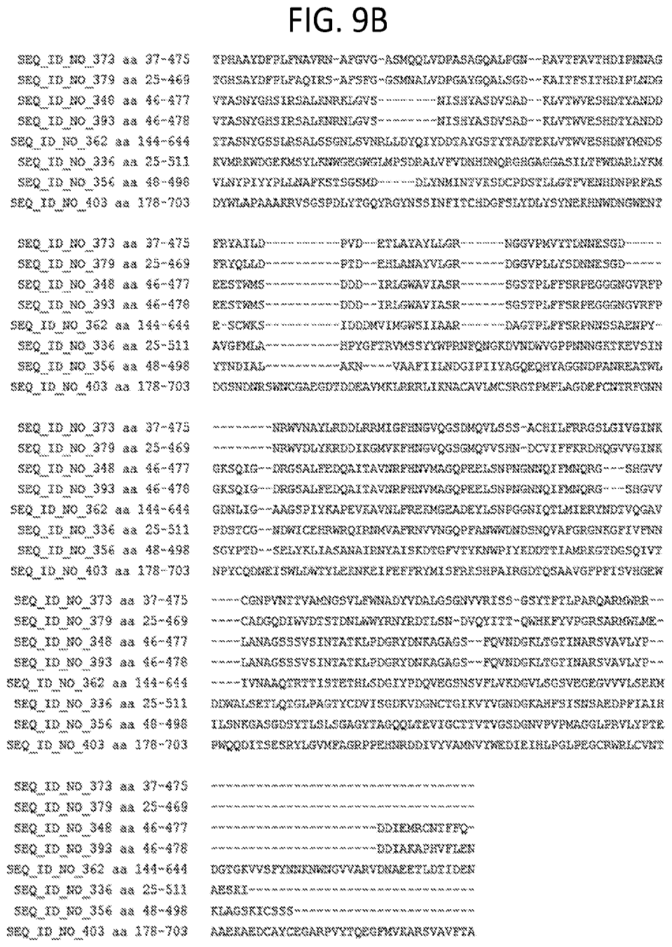

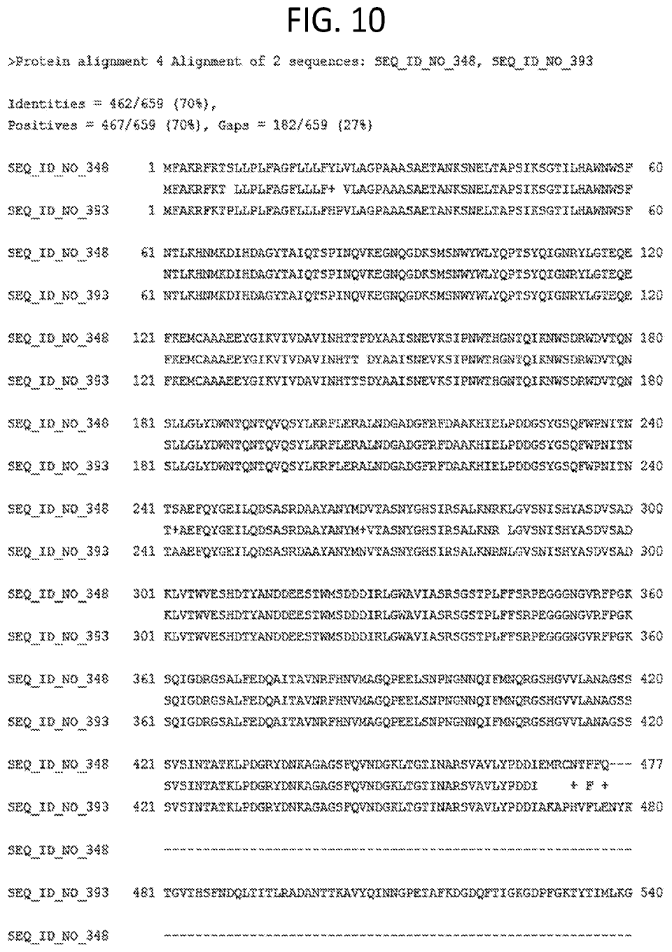

FIGS. 9 and 10 show sequence alignments including some of the amylase polypeptides disclosed as SEQ ID NOs: 327-414. Based on these alignments it becomes immediately apparent to a person of ordinary skill in the art that various amino acid residues may be altered (i.e. substituted, deleted, etc.) without substantially affecting the amylase activity of the polypeptide. For example, a person of ordinary skill in the art would appreciate that substitutions in a reference amylase polypeptide could be based on alternative amino acid residues that occur at the corresponding position in other phytase polypeptides from other species. SEQ ID NOs: 327-414 may also include phytase polypeptides that are not shown in FIGS. 9 and 10. A person of ordinary skill in the art, however, could easily align these polypeptide sequences with the polypeptide sequences shown in FIGS. 9 and 10 to determine what additional variants could be made to the amylase polypeptides.

The heterologous polypeptides produced by the fermentation methods described herein may be one or more pullulanase enzymes. Pullulanase enzymes (EC 3.2.1.41) catalyze the hydrolytic cleavage of alpha-glucan polysaccharides. Examples of pullulanase polypeptides suitable for use in the methods described herein include, but are not limited to, the pullulanase enzymes from Klebsiella pneumoniae. The pullulanase polypeptide may include a sequence at least 50%, 55%, 60%, 65%, 70%, 75%, 80%, 85%, 90%, 95%, 98%, or 99% identical to SEQ ID NO:456.

The fermentation methods described herein are capable of producing additional heterologous biomolecule. For example, an engineered microorganism, during fermentation, can produce a heterologous amino acid, a heterologous antigen, a heterologous cofactor, a heterologous hormone, a heterologous vitamin, a heterologous lipid, a heterologous protein, or a mixture thereof. According to some examples, the amino acid can be an essential amino acid to an animal. Examples of essential amino acid can include lysine, methionine, phenylalanine, threonine, isoleucine, tryptophan, valine, leucine, arginine, taurine, histidine, or mixtures thereof. The amino acids can be produced according to some examples by hydrolyzing the heterologous polypeptides produced to short chain polypeptides or to individual amino acids. Examples of vitamins that can be produced by the engineered microorganism during fermentation can include vitamin A, vitamin B1, vitamin B2, vitamin B3, vitamin B5, vitamin B6, vitamin B7, vitamin B9, vitamin B12, vitamin C, vitamin D1, vitamin D2, vitamin D3, vitamin D4, a tocopherol, vitamin K, or mixtures thereof. Examples of hormones that can be produced by the engineered microorganism during fermentation can include an insulin precursor, a glucagon, or a mixture thereof. Examples of antigens that can be produced by the engineered microorganism during fermentation can include a hepatitis surface antigen. Additional heterologous biomolecules that can be produced can include a heterologous cholesterol, a heterologous prebiotic (e.g., a mannan-oligosaccharide, a fracto-oligosaccharide, or a human milk oligosaccharide) a heterologous antibody, or heterologous immune signaling molecules (e.g., those capable of producing intracrine signals, autocrine signals, juxtracrine signals, paracrine signals, or endocrine signals).

During fermentation, it can be possible for any of the heterologous polypeptides, heterologous amino acids, heterologous antigens, heterologous cofactors, heterologous hormones, heterologous vitamins, heterologous lipids, or heterologous pharmaceuticals to be encapsulated in the engineered microorganism or secreted from the microorganism during fermentation. Whether any of the heterologous biomolecules described herein are capable of being retained by way of encapsulation or secreted can be a function of the engineered microorganism's natural function. Alternatively, the engineered microorganism can be designed in such a manner that secretion of a specific heterologous biomolecule or mixture of heterologous biomolecules can be blocked. Where the one or more heterologous polypeptides are desired to be encapsulated intercellularly, a greater amount of the one or more heterologous polypeptides can be encapsulated than secreted.

Where the heterologous polypeptide has enzymatic activity, the enzymatic activity can be any positive integer. For example, according to various examples the enzymatic activity of the heterologous polypeptide can be in a range of from about 0.1 Units (U)/mg to about 5 U/mg, about 0.2 U/mg to about 1 U/mg, or greater than 0.1 U/mg, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 U/mg. The enzymatic activity of the heterologous polypeptide can be determined through various assays. A unit of enzyme activity is defined as the amount of the enzyme that catalyzes the conversion of one micromole of substrate per minute under the specified conditions of the assay method. One of skill in the art will recognize the appropriate assay method for determining a unit of enzyme activity for a give enzyme.

Following expression in the engineered microorganism, at least a portion of the one or more heterologous polypeptides can be post-translationally modified. Post-translation modification can include acetylation, amidation, hydroxylation, methylation, N-linked glycosylation, O-linked glycosylation, phosphorylation, pyrrolidone carboxylic acid, sulfation, ubiquitylation, or a combination thereof. Post-translational modification can result from the natural activities of the engineered microorganism or can be an engineered result stemming from the modification of the microorganism.

In total, the fermentation process can be run for any suitable amount of time. For example, the fermentation process can be run for an amount of time in a range of from about 0.5 hours to about 72 hours, about 1 hour to about 40 hours, about 2 hours to about 10 hours, less than, equal to, or greater than 0.5 hours, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, or 72 hours. The amount of time that fermentation is run can be controlled. Factors that influence the amount of time that the fermentation reaction is run at include not letting the reaction occur past a point at which bioproduct production, heterologous polypeptide production, or both are reduced or otherwise negatively impacted.

The bioproduct produced by the fermentation process can be distilled to obtain the bioproduct in a substantially purified form. Distillation can be accomplished according to many suitable industrial distillation processes. Distillation can be accomplished by isolating a mixture including the bioproduct from the engineered microorganisms and distilling the desired bioproduct therefrom. Alternatively, distillation can be carried out on a mixture of the bioproduct and the engineered microorganism. In this case, the heterologous polypeptides, or any other heterologous biomolecule encapsulated within the engineered microorganism, can substantially retain their functionality after exposure to a distillation process. The amount of the heterologous biomolecules that can retain their functionality can be in a range of from about 10 wt % to about 100 wt % of the total number of heterologous biomolecules about 20 wt % to about 90 wt %, about 30 wt % to about 50 wt %, less than, equal to, or greater than 10 wt %, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 wt %.

In operation, following fermentation, the heterologous biomolecules such as the heterologous polypeptides can be removed and isolated from the engineered microorganism. Removal can be accomplished naturally through secretion or the engineered microorganism can be lysed and the heterologous polypeptides can be separated from the resulting solution. Lysing can occur, for example, by exposing the engineered microorganism to a detergent that can break a cell membrane or cell wall. Lysing can also occur by introducing an enzyme that is able to cause lysing of the engineered microorganism. Additional methods of causing lysing can include subjecting the engineered microorganism to a suitable pressure to cause the heterologous biomolecules to diffuse from the microorganism via osmosis. Lysing can also occur by introducing strain on the engineered microorganism through one or more freeze-thaw cycles, exposure to ultrasonication, or through homogenization. In some examples, secretion may not be desirable and can occur nevertheless during fermentation. In some examples, where this occurs, the engineered microorganism can be engineered such that the engineered microorganism secretes comparatively less of the one or more heterologous polypeptides than a corresponding naturally occurring microorganism.

According to various further examples, any of the engineered microorganisms that include the heterologous biomolecules or polypeptides can be individually isolated from the solution in which it is disposed. It may be necessary to purify the isolated heterologous biomolecules from other constituents of the engineered microorganisms. Examples of suitable purification techniques can include differential centrifugation, differential salt precipitation, differential solvent precipitation, preparative electrophoresis (e.g., using polyacrylamide gels or isoelectric focusing), column chromatography (e.g., using gel filtration, ion-exchange, or affinity chromatograph), or dialysis.

Once isolated, the engineered microorganism can be combined with a substrate. The substrate can include cellulose, a wood chip, a vegetable, a biomass, animal waste, oat, wheat, corn, barley, milo, millet, rice, rye, sorghum, potato, sugar beets, taro, cassaya, a fruit, a fruit juice, a sugar cane, or mixtures thereof. According to various examples, the substrate can be an animal feed, a pharmaceutical, gasoline or any other desirable substrate. The animal feed can be a feed used for livestock (e.g., cattle, swine, or poultry). The animal feed can also refer to a food product for human consumption. If designed for human consumption, the heterologous biomolecule can be a heterologous polypeptide with beneficial properties for different segments of the human population. Once mixed with the substrate, the encapsulated heterologous biomolecule or heterologous polypeptide can be separated from the engineered microorganism and mixed with the substrate. In effect, the encapsulated heterologous biomolecule can be protected in the engineered microorganism through a fermentation process, distillation process, or both and delivered, intact, to a substrate of interest so as to provide a product having the heterologous polypeptide of interest incorporated therein.

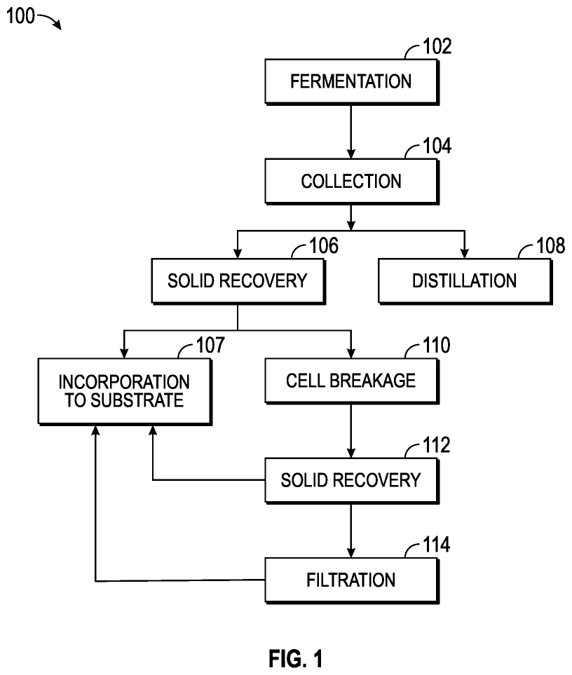

An example of a fermentation process of the instant disclosure is shown in FIG. 1. As shown in FIG. 1, fermentation and processing method 100 is depicted as a flow diagram. At operation 102, an engineered microorganism, such as any of those described herein is subjected to fermentation to produce the heterologous biomolecules and the bioproduct, both of which are collected in a receiver at operation 104. Operation 102 and operation 104 can be performed in the same vessel, e.g., the collection in operation 104 can be optional and the materials resulting from the fermentation in operation 102 (or a portion thereof) can be sent directly to operations 106 and/or 108. The fermentation in operation 102 can be performed in multiple fermentation vessels, operating either in series or in parallel. Following collection at operation 104, the solid engineered microorganisms are separated from the collected fermentation materials at operation 106. As an example, operation 106 can be accomplished using centrifugation. Following centrifugation at operation 106, the liquid supernatant, which can include a bioproduct such as ethanol, is removed and subjected to distillation at operation 108. Alternatively, the bioproduct can be recovered in operation 108 via methods other than distillation, for example via extraction if the bioproduct is a solid or if distillation is not desired to be used for recovery.

The solids recovered at operation 106 can be directly incorporated to a substrate (e.g., animal feed) at operation 107. Alternatively, the solids can be subjected to further processing. For example, at operation 110, the engineered microorganisms in the recovered solids can be subjected to cell breakage. Cell breakage can include lysing. The heterologous bioproduct can be separated from the lysed contents through further centrifugation at operation 112. The heterologous bioproducts can be isolated from the solids obtained at operation 112 through various filtration techniques at operation 114 such as affinity chromatography. The obtained heterologous bioproduct can then be incorporated into the substrate. Fermentation and processing method 100 can also include additional steps, for example purification of the heterologous bioproducts after recovery at operations 106 or 112.

Although FIG. 1 shows separation of the solid engineered microorganism at operation 106 occurring before distillation at operation 108, it is understood that in various examples, operation 108 can occur before operation 106. According to various examples, a reason to have operation 106 occur before operation 108 is to minimize the extent to which any heterologous polypeptides of interest are exposed to high temperatures and possibly denatured as a consequence. However, in examples where operation 108 does occur before operation 106, the risk of denaturization can be mitigated using vacuum distillation. As is understood, vacuum distillation is a method of distillation performed under reduced pressure, which lowers the boiling point of most liquids and reduces or eliminates the need to expose sensitive materials to heating.

With respect to any of the methods and products described herein, the choice of the heterologous polypeptide to be coproduced during fermentation can be made using screening to determine whether a particular heterologous polypeptide is suited for the methods described herein. For example, a strain of any heterologous polypeptide can be screened for enzymatic activity. If the heterologous polypeptide shows enzymatic activity at or above a threshold value, that can indicate that the heterologous polypeptide can be well suited for inclusion in the methods described herein. The enzymatic activity that can be screened for can include screening for intracellular activity as well as for extracellular activity. Additionally, if it is desired for the heterologous polypeptides to be substantially retained within the engineered microorganism, then the heterologous polypeptides can be further screened for their ability be substantially retained intercellularly. Another consideration in choosing the heterologous polypeptide is whether expression of the heterologous polypeptide in the engineered microorganism creates a burden on the production of the bioproduct during fermentation. For example, if production of the one or more heterologous polypeptides brings the production of a bioproduct such as ethanol to an amount below those described herein, then it may not be feasible to go through with producing the heterologous polypeptide. Therefore, it can be desirable to screen heterologous polypeptides for a balance of enzymatic activity and a lack of bioproduct production burden.

### WORKING EXAMPLES

Various embodiments of the present disclosure can be better understood by reference to the following Working Examples which are offered by way of illustration. The present disclosure is not limited to the Working Examples given herein.

Protocols referenced in Working Examples herein are described below.

One unit of Phytase activity will liberate 1.0 μmole of inorganic phosphorus from 4.4 mM phytate per minute at 37° C. at the respective pH. Phytase activity was determined by the following protocol:
1. Prepare fresh phytate substrate (4.4 mM phytic acid sodium salt (Sigma P8810) in 200 mM glycine-HCl buffer, pH 3.5). Prepare from color reagent solution (1 part 5% ammonium molybdate solution, 1 part 5N sulfuric acid and 2 parts acetone).
2. Prepare dilutions of the test samples containing phytase, as appropriate.
3. Each sample tested produces have 2 absorbance readouts: Test and Substrate Blank (SB). There will also be an enzyme blank (EB) well. SB will account for inorganic phosphate already present in the sample. EB accounts for inorganic phosphate released from the phytate substrate under the reaction conditions, for reasons other than phytase action. BB: Blank buffer, B: Blank water.
4. Reaction set-up in 96-well PCR plates:
   Test: 150 μL phytate substrate+15 μL sample;
   SB: 150 μL buffer+15 μL sample;
   EB: 150 μL phytate substrate+15 μL buffer;
   BB: 165 μL buffer; and
   B: 165 μL water
   Incubate the sealed plate at 37° C. in a thermocycler.
5. Prepare 96-well plates (clear, flat-bottom) with 200 μL color reagent solution in each well, for each time-point. Collect 22 μL of the reaction mix at different incubation timepoints (15 min, 30 min, 45 min and so on; a minimum of 4 timepoints) and add to the CRS-containing wells.
6. Record absorbance observations using SpectraMax plate reader at 400 nm wavelength with respect to water blank (B).
7. Prepare a standard curve for inorganic phosphate ranging from 0-10 mM potassium phosphate solution. Different concentrations of inorganic phosphate can be prepared in glycine buffer. 22 μL of each concentration is added to 200 μL of color reagent solution in a 96-well clear flat-bottom plate to develop color and absorbance recorded at 400 nm wavelength.
8. Protein concentration in the samples was estimated using PIERCE®660 nm protein assay reagent (Thermo Scientific Product #22660). 5 μL of sample (as well as BSA solution for standard curve) was added to 195 μL of protein assay reagent in a 96-well clear flat-bottom plate, followed by incubation at room temperature for 5 minutes. Absorbance was recorded at 660 nm using SpectraMax plate reader.
9. Phytase activity calculations:
   Step I: Phosphate standard curve: Plot absorbance at 400 nm reading (Y-axis) versus amount of phosphate in reaction mix (μmoles) and determine slope (S_{P}_{ᵢ}).
   Step II: Protein standard curve: Plot absorbance at 660 nm reading (Y-axis) versus protein concentration (mg/mL) and determine slope (Sₚᵣₒₜₑᵢₙ).
   Step III: Phytase activity in terms of rate of phosphate released (μmoles/min):
      a) Calculate the net absorbance for the test samples as per the following formula:

Net Abs₄₀₀=Test−SB−EB=Abs_{P}_{ᵢ}
b) Determine the amount of inorganic phosphate for each sample, at each incubation timepoint, using the slope from the phosphate standard curve.

Amount of phosphate released (μmoles)=Abs_{P}_{ᵢ}/S_{P}_{ᵢ}
c) Plot the amount of phosphate released (μmoles) (Y-axis) versus assay time (min) and determine slope (r_{P}_{ᵢ}, μmoles/min).
Step IV: Normalize phytase activity with respect to total protein, used for the reaction, to obtain phytase activity in terms of rate of phosphate released per mg protein (μmoles/min·mg):
   a) Determine protein concentration in each sample using slope from the protein standard curve as follows:

Sample protein concentration (Cₚᵣₒₜₑᵢₙ, mg/mL)=Net Abs₆₆₀/Sₚᵣₒₜₑᵢₙ
b) Determine amount of protein used in the reaction for each sample, i.e. amount of protein in 2 μL (volume of sample in the phosphate estimation reaction):

Amount of protein used for reaction (mg)=Cₚᵣₒₜₑᵢₙ*2/1000=mₚᵣₒₜₑᵢₙ
c) Determine normalized phytase activity (μmoles/min·mg), by dividing the rate of Pᵢ released by amount of protein in the reaction volume.

Phytase activity (μmoles/min·mg)=r_{P}_{ᵢ}/mₚᵣₒₜₑᵢₙ
10. Phytase activity at pH 5.5 was determined by replacing the glycine buffer in the phytate substrate buffer with 200 mM Citrate-HCl, pH 5.5.

Amylase activity will liberate 1.0 μmole of reducing sugar from a 1% starch solution per minute at 30° C. at the pH 5.6. Amylase activity was determined by the following protocol:
1. Prepare fresh 1% starch solution by dissolving 1 g of soluble starch in 100 mls of 90° C. water, allow to cool to room temperature, and add 2 mls of 3M NaOAc.
2. Prepare fresh DNS reagent by dissolving 1 g 3,5-dinitrosalicylic acid, 30 g sodium potassium tartrate tetrahydrate, 20 mls of 2N NaOH in 30 mls of water. Bring up to 100 mls total volume.
3. Prepare a fresh glucose standard by dissolving 3 g D-Glucose monohydrate (Sigma #49161) in 1 L of water. Make a series of six 1:1 dilutions in water.
4. Prepare dilutions of the test samples containing amylase, as appropriate.
5. Each sample tested will have 2 absorbance readouts: Test and Substrate Blank (SB). SB will account for glucose already present in the sample. Enzyme blank will serve as the enzyme blank, to account for any glucose present in the starch solution (EB). Water serves as the plate blank (B).
6. Reaction set-up in 96-well PCR plates:
   Test: 100 μL 1% starch solution+50 μL sample+50 μL water
   SB: 150 μL water+50 μL sample
   EB: 150 μL 1% starch solution+50 μL buffer
   B: 200 μL water
7. Incubate the plate at 30° C. for approximately one hour
8. In a separate 96-well PCR plate add 75 μL of DNS reagent to each well. Remove 75 μL of the starch assay mix and add to the 75 μL of DNS and incubate at 99° C. for 10 minutes.
9. Remove 100 μL and place in a new flat bottom 96-well plate and read absorbance at 540 nm.
10. Protein concentration in the samples was estimated using PIERCE®660 nm protein assay reagent (Thermo Scientific Product #22660). 5 μL of sample (as well as BSA solution for standard curve) was added to 195 μL of protein assay reagent in a 96-well clear flat-bottom plate, followed by incubation at room temperature for 5 minutes. Absorbance was recorded at 660 nm using SpectraMax plate reader.
11. Amylase activity calculations:
   Step I: Glucose standard curve: Plot absorbance at 540 nm reading (Y-axis) versus amount of glucose in reaction mix (μmoles) and determine slope (S_{P}_{ᵢ}).
   Step II: Protein standard curve: Plot absorbance at 660 nm reading (Y-axis) versus protein concentration (mg/mL) and determine slope (Sₚᵣₒₜₑᵢₙ).
   Step III: Amylase activity in terms of rate of glucose released (μmoles/min):
      d) Calculate the net absorbance for the test samples as per the following formula:

Net Abs₄₀₀=Test−EB−SB=Abs_{P}_{ᵢ}
e) Determine the amount of glucose for each sample, at each incubation timepoint, using the slope from the glucose standard curve.

Amount of glucose released (μmoles)=Abs_{P}_{ᵢ}/S_{P}_{ᵢ}
f) Plot the amount of glucose released (μmoles) (Y-axis) versus assay time (min) and determine slope (r_{P}_{ᵢ}, μmoles/min).
Step IV: Normalize amylase activity with respect to total protein, used for the reaction, to obtain amylase activity in terms of rate of reducing sugar released per mg protein (μmoles/min·mg):
   d) Determine protein concentration in each sample using slope from the protein standard curve as follows:

Sample protein concentration (Cₚᵣₒₜₑᵢₙ, mg/mL)=Net Abs₆₆₀/Sₚᵣₒₜₑᵢₙ
e) Determine amount of protein used in the reaction for each sample, i.e. amount of protein in 2 μL (volume of sample in the phosphate estimation reaction):

Amount of protein used for reaction (mg)=Cₚᵣₒₜₑᵢₙ*2/1000=mₚᵣₒₜₑᵢₙ
f) Determine normalized amylase activity (μmoles/min·mg), by dividing the rate of glucose released by amount of protein in the reaction volume.

Amylase activity (μmoles/min·mg)=r_{P}_{ᵢ}/mₚᵣₒₜₑᵢₙ

One unit of Pullanase activity will liberate 1.0 μmole of reducing sugar from 1% pullulan solution per minute at 30° C. Pullulanase activity was determined by the following protocol:
1. Prepare fresh 1% pullulan solution by dissolving 1 g of pullulan in 20 mM pH 6.9 sodium phosphate buffer with 6.7 mM sodium chloride.
2. Prepare fresh DNS reagent by dissolving 12 g sodium potassium tartrate tetrahydrate in 8 mL 2M NaOH, 20 mL 96 mM 3,5-dinitrosalicyclic acid, 12 mL water.
3. Prepare a fresh glucose standard by dissolving 3 g D-Glucose monohydrate (Sigma #49161) in 1 L of water. Make a series of six 1:1 dilutions in water.
4. Prepare dilutions of the test samples containing pullulanase, as appropriate.
5. Each sample tested will have 2 absorbance readouts: Test and Substrate Blank (SB). SB will account for glucose already present in the sample. Enzyme blank will serve as the enzyme blank, to account for any glucose present in the pullulan solution (EB). Water serves as the plate blank (B).
6. Reaction set-up in 96-well plates:
   Test: 20 μL 1% pullulan solution+5 μL sample
   SB: 20 μL water+5 μL sample
   EB: 20 μL 1% pullulan solution+5 μL buffer
   B: 25 μL water
7. Incubate the plate at 30° C. for 5 minutes (time may vary)
8. Add 96-well PCR plate add 225 μL of DNS reagent to each well, and read absorbance at 540 nm.
9. Protein concentration in the samples was estimated using PIERCE®660 nm protein assay reagent (Thermo Scientific Product #22660). 5 μL of sample (as well as BSA solution for standard curve) was added to 195 μL of protein assay reagent in a 96-well clear flat-bottom plate, followed by incubation at room temperature for 5 minutes. Absorbance was recorded at 660 nm using SpectraMax plate reader.
10. Pullulanase activity calculations:
   Step I: Glucose standard curve: Plot absorbance at 540 nm reading (Y-axis) versus amount of glucose in reaction mix (μmoles) and determine slope (S_{P}_{ᵢ}).
   Step II: Protein standard curve: Plot absorbance at 660 nm reading (Y-axis) versus protein concentration (mg/mL) and determine slope (Sₚᵣₒₜₑᵢₙ).
   Step III: Pullulanase activity in terms of rate of glucose released (μmoles/min):
      g) Calculate the net absorbance for the test samples as per the following formula:

Net Abs₄₀₀=Test−EB−SB=Abs_{P}_{ᵢ}
h) Determine the amount of glucose for each sample, at each incubation timepoint, using the slope from the glucose standard curve.

Amount of glucose released (μmoles)=Abs_{P}_{ᵢ}/S_{P}_{ᵢ}
i) Plot the amount of glucose released (μmoles) (Y-axis) versus assay time (min) and determine slope (r_{P}_{ᵢ}, μmoles/min).
Step IV: Normalize Pullulanase activity with respect to total protein used for the reaction, to obtain pullulanase activity in terms of rate of reducing sugar produced per mg protein (μmoles/min·mg):
   g) Determine protein concentration in each sample using slope from the protein standard curve as follows:

Sample protein concentration (Cₚᵣₒₜₑᵢₙ, mg/mL)=Net Abs₆₆₀/Sₚᵣₒₜₑᵢₙ
h) Determine amount of protein used in the reaction for each sample, i.e. amount of protein in 2 μL (volume of sample in the phosphate estimation reaction):

Amount of protein used for reaction (mg)=Cₚᵣₒₜₑᵢₙ*2/1000=mₚᵣₒₜₑᵢₙ
i) Determine normalized pullulanase activity (μmoles/min·mg), by dividing the rate of glucose released by amount of protein in the reaction volume.

Pullulanase activity (μmoles/min·mg)=r_{P}_{ᵢ}/mₚᵣₒₜₑᵢₙ

### Working Example #1. Saccharomyces cerevisiae Base Strain Construction

Strain 1-3, described in International Patent Application Publication No. WO 2016/160584, filed 25 Mar. 2016, is a Saccharomyces cerevisiae strain (Strain 14883, a version of Ethanol Red™ Saccharomyces cerevisiae) in which both copies of the URA3 gene are deleted. For the purposes of this disclosure, strain 1-3 is referred to as Strain 1.

### Working Example #2. Constructing a Haploid Ura3Δ Saccharomyces cerevisiae Strain

Strain 1 was cultured for 24 hours at 30° C. on plates containing 5% Dextrose, 3% Difco Nutrient Broth, 1% yeast extract, 2% Bacto Agar and 2 mg/ml uracil. A small amount of cells was transferred to 10 ml of Liquid Sporulation Media containing 1% KOAc, 0.005% ZnOAc, pH 7.0, 2.5 μL/ml of a uracil stock solution (2 mg/ml) and incubated at 200 RPM at 23° C. for 1 week. Four spore tetrads were dissected and arrayed onto YPD plates. Mating type for each haploid was determined using standard methods. A MATa haploid was designated Strain 2.

### Working Example #3. Constructing a Haploid Ura3Δ Leu2Δ Saccharomyces Cerevisiae Strain

Strain 2 was transformed with SEQ ID NO: 1. SEQ ID NO: 1 contains i) homology upstream of the ScLEU2 locus, ii) an expression cassette for a isomaltose transporter from Saccharomyces mikatae, iii) homology downstream of the LEU2 locus. Transformants were selected on YNB+1% isomaltose. Resulting transformants were struck to similar media for single colony isolation. Correct integration of SEQ ID NO 1: into the ScLEU2 locus is verified by PCR. A PCR verified isolate was designated Strain 3.

### Working Example #4. Transformation of Phytase Library in Strain 1

Strain 1 was transformed with SEQ ID NO 2: in combination with one or more of SEQ ID NO: 3 through SEQ ID NO: 103. As outlined in Table 1, SEQ ID NO: 3 through SEQ ID NO: 103 can be transformed individually or in combination with another sequence selected from SEQ ID NOs: 3-103. SEQ ID NO: 2 contains i) an empty expression cassette containing the TDH3 promoter and ScCYC1 terminator; ii) a ScURA3 expression cassette; iii) the Saccharomyces cerevisiae CEN6 centromere for stable replication; and iv) a beta-lactamase expression cassette. SEQ ID NO: 3 through SEQ ID NO: 103 contain individual phytase homologues, or parts of a phytase homologue, codon optimized for Saccharomyces cerevisiae with an additional 45 bp on the 5′ and 3′ ends that are homologous to the 5′ and 3′ ends on SEQ ID NO: 2 to facilitate shuttle vector cloning. In some cases more than a single SEQ ID per phytase was transformed as the phytase was broken into two gene fragments due to DNA synthesis length requirements. Transformants were selected on synthetic dropout media lacking uracil (ScD-Ura). A representative strain from each was saved as Strain 1-1 through Strain 1-87

### Working Example #5. Phytase Activity Analysis in Strains Harboring Phytase Library

A patch of cells from a ScD-Ura plate containing Strain 1-1 through Strain 1-87 was used to inoculate a 15 mL falcon tube containing 3 mL of ScD-Ura media (6.7 g/L yeast nitrogen base, 1.9 g/L ScD-Ura drop out mix, 100 g/L glucose, and 0.1M MES pH 6.0) and grown for 16 hours at 32° C. with an agitation of 250 RPM. The cultures were harvested by centrifuging at 4000 RPM, 4° C. for 10 minutes, followed by a wash with ice cold water and storage of pellets at −80° C. For the phytase assay, the pellets were thawed on ice, resuspended in 500 μL lysis buffer (50 mM Tris HCl buffer pH 6.8 containing 1% β-mercaptoethanol and 1× Halt™ Protease inhibitor cocktail) and transferred to mechanical lysis plate containing 500μ glass beads. Two bead beating cycles were carried out for lysis and cell free lysate was collected after centrifuging at 6000 RPM for 5 minutes. Phytase activity was determined using the protocol described above. Phytase activity was determined at both pH 3.5 and 5.5, the results of which are compiled in Table 1. At pH 3.5, 50 strains tested positive for phytase activity, while at pH 5.5 64 strains tested positive.

Phytases with the highest activities when expressed in Strain 1 are sourced from (including polypeptide sequence) Azorhizobium caulinodans (SEQ ID NO 273), Citrobacter freundii (SEQ ID NO 267), Thermomyces lanuginosus(SEQ ID NO 244), Aspergillus awamori (SEQ ID NO 252), Citrobacter werkmanii (SEQ ID NO 268), Kosakonia sacchari (SEQ ID NO 269), Peniophora lycii (SEQ ID NO 246), Citrobacter amalonaticus (SEQ ID NO 270), Citrobacter braakii (SEQ ID NO 326), Cronobacter sakazakii (SEQ ID NO 271), and Aspergillus japonicus (SEQ ID NO 311).

TABLE 1
Strain ID, SEQ ID, Phytase source, Phytase activity pH 3.5, activity pH 5.5

| | | | Phytase | Phytase |
|---|---|---|---|---|
| | Corresponding | | Activity @ pH 3.5 | Activity @ pH 5.5 |
| Strain ID | SEQ ID | Phytase source | (umoles/min · mg) | (umoles/min · mg) |
| Strain 1-1 | SEQ ID NO: 3 | Rhizomucor pusillus | 0.040 | 0.023 |
| Strain 1-2 | SEQ ID NO: 4 | Arabidopsis thaliana | 0.002 | 0.002 |
| Strain 1-3 | SEQ ID NO: 5 | Saccharomyces mikatae | 0.007 | 0.008 |
| Strain 1-4 | SEQ ID NO: 6 | A. fumigatus | 0.015 | 0.031 |
| Strain 1-5 | SEQ ID NO: 7 | Thermomyces lanuginosus | 0.041 | 0.128 |
| Strain 1-6 | SEQ ID NO: 8 | A. niger 3-phytase A | 0.003 | 0.008 |
| Strain 1-7 | SEQ ID NO: 9 | Peniophora lycii | 0.129 | 0.200 |
| Strain 1-8 | SEQ ID NO: 10 | Agrocybe pediades | 0.030 | 0.047 |
| Strain 1-9 | SEQ ID NO: 11 | Ceriporia sp. | −0.002 | 0.014 |
| Strain 1-10 | SEQ ID NO: 12 | Trametes pubescens | 0.019 | 0.090 |
| Strain 1-11 | SEQ ID NO: 13 | Bacillus licheniformis | −0.004 | −0.009 |
| Strain 1-12 | SEQ ID NO: 14 | Bacillus sp. DS11 | 0.015 | −0.005 |
| Strain 1-13 | SEQ ID NO: 15 | Aspergillus awamori | 0.114 | 0.217 |
| Strain 1-14 | SEQ ID NO: 16 | Aspergillus flavus | 0.002 | 0.001 |
| Strain 1-15 | SEQ ID NO: 17 | B. amyloliquefaciens | −0.003 | −0.001 |
| Strain 1-16 | SEQ ID NO: 18 | Pectobacterium carotovorum | −0.002 | −0.031 |
| Strain 1-17 | SEQ ID NO: 19 | Lupinus albus | 0.020 | −0.001 |
| Strain 1-18 | SEQ ID NO: 20 | Pediococcus pentosaceus | 0.002 | 0.003 |
| Strain 1-19 | SEQ ID NO: 21 | Pediococcus claussenii | 0.003 | 0.001 |
| Strain 1-20 | SEQ ID NO: 22 | Magnaportha grisea | 0.010 | 0.002 |
| Strain 1-21 | SEQ ID NO: 23 | Pediococcus pentosaceus | 0.005 | 0.003 |
| Strain 1-22 | SEQ ID NO: 24 | Lactobacillus rhamnosus GG | 0.019 | −0.003 |
| Strain 1-23 | SEQ ID NO: 25 | Lactobacillus casei | 0.004 | −0.009 |
| Strain 1-24 | SEQ ID NO: 26 | Lactococcus lactis | −0.007 | 0.001 |
| Strain 1-25 | SEQ ID NO: 27 | Lactobacillus amylovorus | 0.031 | −0.004 |
| Strain 1-26 | SEQ ID NO: 28 | Rhizopus microsporas | 0.006 | −0.003 |
| Strain 1-27 | SEQ ID NO: 29 | Mus musculus | −0.006 | −0.003 |
| Strain 1-28 | SEQ ID NO: 30 | Citrobacter freundii | 0.153 | 0.136 |
| Strain 1-29 | SEQ ID NO: 31 | Citrobacter werkmanii | 0.213 | 0.266 |
| Strain 1-30 | SEQ ID NO: 32 | Kosakonia sacchari | 0.075 | 0.132 |
| Strain 1-31 | SEQ ID NO: 33 | Citrobacter amalonaticus | 0.061 | 0.145 |
| Strain 1-32 | SEQ ID NO: 34 | Cronobacter sakazakii | 0.014 | 0.106 |
| Strain 1-33 | SEQ ID NO: 35 | Cronobacter turicensis | 0.027 | 0.014 |
| Strain 1-34 | SEQ ID NO: 36 | Azorhizobium caulinodans | 0.267 | −0.008 |
| | | ORS 571 | | |
| Strain 1-35 | SEQ ID NO: 37 | Xanthomonas campestris pv. | −0.012 | 0.001 |
| | | raphani 756C | | |
| Strain 1-36 | SEQ ID NO: 38 | Azospirillum sp. B510 | 0.006 | −0.003 |
| Strain 1-37 | SEQ ID NO: 39 | Periplasmic appA protein | 0.034 | 0.025 |
| | | precursor [Escherichia coli | | |
| | | CFT073] | | |
| Strain 1-38 | SEQ ID NO: 40 | Phytase AppA [Escherichia | 0.076 | 0.095 |
| | | albertii)] | | |
| Strain 1-39 | SEQ ID NO: 41 | Candidatus Hamiltonella | −0.010 | −0.010 |
| | | defensa | | |
| Strain 1-40 | SEQ ID NO: 42 | Edwardsiella ictaluri 93-146 | 0.003 | 0.001 |
| Strain 1-41 | SEQ ID NO: 43 | Agrocybe pediades | 0.083 | 0.031 |
| Strain 1-42 | SEQ ID NO: 44 | Myroides odoratus | 0.015 | −0.016 |
| Strain 1-43 | SEQ ID NO: 45 | Advenella Kashmirensis | 0.004 | −0.027 |
| Strain 1-44 | SEQ ID NO: 46 | Penicillium oxalicum | 0.006 | −0.001 |
| Strain 1-45 | SEQ ID NO: 47 | Cladosporium | 0.018 | 0.004 |
| Strain 1-46 | SEQ ID NO: 48 | Peniophora lycii | 0.012 | 0.016 |
| Strain 1-47 | SEQ ID NO: 49 | Parcubacteria group bacterium | −0.004 | 0.013 |
| Strain 1-48 | SEQ ID NO: 50, 90 | Streptomyces fradiae | 0.010 | 0.009 |
| Strain 1-49 | SEQ ID NO: 51, 91 | Natranaerobius thermophilus | 0.015 | −0.049 |
| Strain 1-50 | SEQ ID NO: 52, 92 | Marinomonas posidonica | −0.014 | −0.002 |
| Strain 1-51 | SEQ ID NO: 53, 93 | Leptotrichia buccalis | −0.013 | −0.008 |
| Strain 1-52 | SEQ ID NO: 54, 94 | Rhodothermus marinus | −0.009 | 0.001 |
| Strain 1-53 | SEQ ID NO: 55 | Sphaerobacter thermophilus | −0.014 | 0.014 |
| Strain 1-54 | SEQ ID NO: 56, 95 | Thermotoga petrophila | 0.015 | −0.004 |
| Strain 1-55 | SEQ ID NO: 57 | Paenibacillus sp. | −0.002 | 0.000 |
| Strain 1-56 | SEQ ID NO: 58 | Microbulbifer aggregans | −0.018 | 0.000 |
| Strain 1-57 | SEQ ID NO: 59, 96 | Dethiobacter alkaliphilus | 0.013 | 0.024 |
| Strain 1-58 | SEQ ID NO: 60 | Salinispora tropica | −0.010 | 0.011 |
| Strain 1-59 | SEQ ID NO: 61 | Arenibacter algicola | −0.025 | 0.021 |
| Strain 1-60 | SEQ ID NO: 62 | Proteiniborus sp. | −0.004 | 0.015 |
| Strain 1-61 | SEQ ID NO: 63 | Marinimicrobia bacterium | 0.008 | 0.032 |
| Strain 1-62 | SEQ ID NO: 64 | Halorhabdus tiamatea | −0.037 | 0.015 |
| Strain 1-63 | SEQ ID NO: 65 | Streptomyces pratensis | 0.004 | 0.016 |
| Strain 1-64 | SEQ ID NO: 66 | Geobacter uraniireducens | −0.002 | 0.018 |
| Strain 1-65 | SEQ ID NO: 67, 97 | Streptomyces fulvissimus | 0.020 | 0.006 |
| Strain 1-66 | SEQ ID NO: 68 | Rubellimicrobium | −0.003 | 0.003 |
| | | mesophilum | | |
| Strain 1-67 | SEQ ID NO: 69, 98 | Akkermansia muciniphila | −0.010 | 0.007 |
| Strain 1-68 | SEQ ID NO: 70 | Beggiatoa sp. | −0.023 | 0.009 |
| Strain 1-69 | SEQ ID NO: 71 | Pseudomonas fluorescens | −0.014 | 0.002 |
| Strain 1-70 | SEQ ID NO: 72 | Aspergillus oryzae | −0.021 | 0.021 |
| Strain 1-71 | SEQ ID NO: 73 | Talaromyces thermophilus | −0.002 | 0.039 |
| Strain 1-72 | SEQ ID NO: 74 | Aspergillus japonicus | 0.073 | 0.160 |
| Strain 1-73 | SEQ ID NO: 75 | Streptomyces sp. | 0.002 | −0.008 |
| Strain 1-74 | SEQ ID NO: 76, 99 | hydrothermal vent | −0.025 | 0.008 |
| | | metagenome | | |
| Strain 1-75 | SEQ ID NO: 77, 100 | Thielavia heterothallica | 0.013 | 0.062 |
| Strain 1-76 | SEQ ID NO: 78 | Pseudomonas mendocina | −0.023 | 0.008 |
| Strain 1-77 | SEQ ID NO: 79, 101 | Cellvibrio sp. | −0.009 | 0.016 |
| Strain 1-78 | SEQ ID NO: 80, 102 | Asticcacaulis biprosthecum | −0.018 | 0.004 |
| Strain 1-79 | SEQ ID NO: 81 | Marinomonas gallaica | −0.014 | 0.009 |
| Strain 1-80 | SEQ ID NO: 82, 103 | Marinomonas mediterranea | −0.012 | 0.001 |
| Strain 1-81 | SEQ ID NO: 83 | Thermobaculum terrenum | 0.008 | 0.012 |
| Strain 1-82 | SEQ ID NO: 84 | Moraxella catarrhalis | −0.014 | 0.022 |
| Strain 1-83 | SEQ ID NO: 85 | Erythrobacter gangjinensis | −0.009 | 0.024 |
| Strain 1-84 | SEQ ID NO: 86 | Novosphingobium sp. | −0.008 | 0.033 |
| Strain 1-85 | SEQ ID NO: 87 | Lilium longiflorum | −0.019 | 0.023 |
| Strain 1-86 | SEQ ID NO: 88 | Bacillus mycoides | −0.013 | 0.006 |
| Strain 1-87 | SEQ ID NO: 89 | Citrobacter braakii | 0.420 | 0.270 |

### Working Example #6. Transformation of Amylase Library in Strain 1

Strain 1 was transformed with SEQ ID NO: 2 in combination with one or more of SEQ ID NO: 104 through SEQ ID NO: 233 (described in Table 2). SEQ ID NO: 2 contains i) an empty expression cassette containing the TDH3 promoter and ScCYC1 terminator; ii) a ScURA3 expression cassette; iii) the Saccharomyces cerevisiae CEN6 centromere for stable replication; and iv) a beta-lactamase expression cassette. SEQ ID NO: 104 through SEQ ID NO: 233 contain individual amylase homologues, or parts of individual homologues, codon optimized for Saccharomyces cerevisiae with an additional 45 bp on the 5′ and 3′ ends that are homologous to the 5′ and 3′ ends on SEQ ID NO: 2 to facilitate shuttle vector cloning. In some cases more than a single SEQ ID per amylase was transformed as the amylase was broken into two gene fragments due to DNA synthesis length requirements. Transformants were selected on synthetic dropout media lacking uracil (ScD-Ura). A representative strain from each was saved as Strain 1-88 through Strain 1-177

### Working Example #7a. Amylase Activity in Strains Expressing Amylase Library

Strains 1-88 through 1-175 were grown overnight in 300 μL of ScD-Ura (6.7 g/L yeast nitrogen base, 1.9 g/L ScD-Ura drop out mix, 100 g/L glucose, and 0.1M MES pH 6.0) per well, in a 96 deep well plate. The plate was placed in an anti-evaporation box and grown overnight at 400 RPM and 30° C. with 80% humidity in an Infors shaker for 24 hours. From this plate, 6 μL of the cells was transferred to another plate containing 600 μL of media and incubated overnight with the same conditions. The following morning the cells were centrifuged at 4000 RPM, washed with ice-cold water prior to freezing the cell pellet at −80 C. Cell pellets were thawed on ice, and resuspended in 500 μL of ice-cold lysis buffer (100 mM potassium phosphate buffer pH 7.0 1× Halt™ Protease inhibitor cocktail). An equivalent volume of glass beads (500μ, Sigma G8772) was added. Cells were lysed in a bead mill, including three 1 minute cycles with 1 minute on ice between cycles, followed by centrifugation at 4° C., 14000 RPM for 10 minutes, from which the cell free lysate was collected prior to analysis. Alpha amylase activity was determined using the protocol described above.

Table 2 describes the Strain ID, corresponding SEQ ID NOs for the specific amylase, the amylase source, the resulting alpha amylase activity, and the associated ethanol titer (See Working Example #7b). The top ten highest alpha amylase activities, having at least 0.2 U/mg or higher belong to (including the polypeptide sequence): Alteromonus macleodii (SEQ ID NO 379), Streptococcus mutans (SEQ ID NO 375), Mus musculus (SEQ ID NO 336), Xanthomonus campestris (SEQ ID NO 373), Bacillus amyloliquiefaciens (SEQ ID NO 393), Bacillus sp. (SEQ ID NO 378), Butyrivibrio fibrisolvens (SEQ ID NO 362), Bacillus subtilis (SEQ ID NO 348), Aspergillus niger (SEQ ID NO 356), and Blautia schinkii (SEQ ID NO 403).

### Working Example #7b. Ethanol Production for Strains Expressing Amylase Library

Strains 1-88 through 1-175 were inoculated into 96 deep well plates containing 600 uL of ScD-Ura media (6.7 g/L yeast nitrogen base, 1.9 g/L ScD-Ura drop out mix, 50 g/L glucose). Plates were incubated overnight at 30° C. and 800 RPM in a Infors HT plate shaker. Seed cultures were then checked for growth by measuring OD600 and culture was transferred to 96 well deep well plates containing 600 uL of media to a target OD600 of 0.2. The shake flask medium includes of 590 g partially hydrolyzed corn starch, 180 g filtered light steep water, 158 g water, 72 g 50% glucose (w/v). Plates were incubated for 24 hrs at 30° C., 800 RPM in a Infors HT plate shaker. Plates were then pulled out of the shaker after 24 hrs and analyzed for ethanol by HPLC. Glucoamylase was not included in this assay, therefore saccharification was done by the expressed amylase. It is expected that addition of a glucoamylase will increase ethanol titer in an equivalent assay.

TABLE 2
Strain ID, SEQ ID, Amylase source, Amylase activity, Ethanol Titer.

| | | | | Ethanol |
|---|---|---|---|---|
| | Corresponding | | Amylase Activity | titer |
| Strain ID | SEQ ID | Amylase source | (umoles/min · mg) | (g/L) |
| Strain 1-88 | SEQ ID NO: 104 | >AAM48112.1 ALPHA-AMYLASE PRECURSOR | 0.0065 | 23.37 |
| | | [THERMOCOCCUS SP. GU5L5] | | |
| Strain 1-89 | SEQ ID NO: 105 | >BAD83655.1 ALPHA-AMYLASE | 0.0012 | 22.18 |
| | | [CALLOSOBRUCHUS CHINENSIS] | | |
| Strain 1-90 | SEQ ID NO: 106 | >CAZ78877.1 UNNAMED PROTEIN PRODUCT | 0.0155 | 26.99 |
| | | [XANTHOMONAS CAMPESTRIS] | | |
| Strain 1-91 | SEQ ID NO: 107 | >ABL75259.1 ALPHA-AMYLASE [BACILLUS | n.d. | n.d. |
| | | LICHENIFORMIS] | | |
| Strain 1-92 | SEQ ID NO: 108, | >AAC49622.1 ALPHA-AMYLASE [LIPOMYCES | 0.0011 | 21.63 |
| | SEQ ID NO: 192 | SPENCERMARTINSIAE] | | |
| Strain 1-93 | SEQ ID NO: 109, | >AAB18548.1 ALPHA-AMYLASE [ESCHERICHIA | 0.0058 | 21.37 |
| | SEQ ID NO: 193 | COLI STR. K-12 SUBSTR. MG1655] | | |
| Strain 1-94 | SEQ ID NO: 110, | >ACD93218.3 ALPHA-AMYLASE [BACILLUS SP. | 0.0035 | 21.53 |
| | SEQ ID NO: 194 | KR-8104] | | |
| Strain 1-95 | SEQ ID NO: 111 | >CAA03110.1 UNNAMED PROTEIN PRODUCT | 0.0029 | 21.18 |
| | | [THERMOMYCES LANUGINOSUS] | | |
| Strain 1-96 | SEQ ID NO: 112, | >AAB60935.1 AMYLASE [AEDES AEGYPTI] | 0.0036 | 21.02 |
| | SEQ ID NO: 195 | | | |
| Strain 1-97 | SEQ ID NO: 113 | >BAC26543.1 UNNAMED PROTEIN PRODUCT | 0.5402 | 21.37 |
| | | [MUS MUSCULUS] | | |
| Strain 1-98 | SEQ ID NO: 114 | >ACF88424.1 UNKNOWN [ZEA MAYS] | 0.0077 | 21.23 |
| Strain 1-99 | SEQ ID NO: 115, | >EPS26265.1 ALPHA-AMYLASE AMY13A | 0.1267 | 23.56 |
| | SEQ ID NO: 196 | [PENICILLIUM OXALICUM 114-2] | | |
| Strain 1-100 | SEQ ID NO: 116 | >AAX84031.1 AMYF [BACILLUS SP. WS06] | 0.0056 | 23.46 |
| Strain 1-101 | SEQ ID NO: 117, | >AAD47072.1 ALPHA-AMYLASE PRECURSOR | 0.0043 | 21.12 |
| | SEQ ID NO: 197 | (PLASMID) [CLOSTRIDIUM ACETOBUTYLICUM | | |
| | | ATCC 824] | | |
| Strain 1-102 | SEQ ID NO: 118, | >NP_342630.1 HYPOTHETICAL PROTEIN SSO1172 | 0.0044 | 21.82 |
| | SEQ ID NO: 198 | [SULFOLOBUS SOLFATARICUS P2] | | |
| Strain 1-103 | SEQ ID NO: 119, | >CAB06816.1 AMLC [STREPTOMYCES LIVIDANS] | n.d. | n.d. |
| | SEQ ID NO: 199 | | | |
| Strain 1-104 | SEQ ID NO: 120 | >AAG60011.1 ALPHA-AMYLASE PRECURSOR | n.d. | n.d. |
| | | [DROSOPHILA YAKUBA] | | |
| Strain 1-105 | SEQ ID NO: 121 | >BAA00336.1 TAKA-AMYLASE A PRECURSOR | 0.0071 | 21.28 |
| | | [ASPERGILLUS ORYZAE] | | |
| Strain 1-106 | SEQ ID NO: 122, | >BAF93484.1 ALKALINE AND THERMOSTABLE | 0.0054 | 21.02 |
| | SEQ ID NO: 200 | AMYLASE [BACILLUS HALODURANS] | | |
| Strain 1-107 | SEQ ID NO: 123, | >AAD45245.1 ALPHA-AMYLASE PRECURSOR | 0.0000 | 6.61 |
| | SEQ ID NO: 201 | [LACTOBACILLUS MANIHOTIVORANS] | | |
| Strain 1-108 | SEQ ID NO: 124 | >CAZ78870.1 UNNAMED PROTEIN PRODUCT | 0.0022 | 21.96 |
| | | [BACILLUS SP.] | | |
| Strain 1-109 | SEQ ID NO: 125 | >CAA26086.1 UNNAMED PROTEIN PRODUCT | 0.2790 | 52.45 |
| | | [BACILLUS SUBTILIS] | | |
| Strain 1-110 | SEQ ID NO: 126 | >CAZ78892.1 UNNAMED PROTEIN PRODUCT | 0.0043 | 22.48 |
| | | [DICTYOGLOMUS THERMOPHILUM] | | |
| Strain 1-111 | SEQ ID NO: 127, | >AAK17994.1 ALPHA-AMYLASE 4 | 0.0307 | 21.27 |
| | SEQ ID NO: 202 | [PSEUDOMONAS SP. KFCC10818] | | |
| Strain 1-112 | SEQ ID NO: 128, | >CAB06622.1 ALPHA-AMYLASE [STREPTOMYCES | 0.0047 | 21.45 |
| | SEQ ID NO: 203 | LIVIDANS] | | |
| Strain 1-113 | SEQ ID NO: 129 | >AGL50368.1 GLYCOGEN BRANCHING ENZYME, | 0.0035 | 21.22 |
| | | GH-57-TYPE, ARCHAEAL [THERMOTOGA | | |
| | | MARITIMA MSB8] | | |
| Strain 1-114 | SEQ ID NO: 130 | >ABU98335.1 ALPHA-AMYLASE | 0.0804 | 21.92 |
| | | [THERMOCOCCUS SP. HJ21] | | |
| Strain 1-115 | SEQ ID NO: 131 | >AAC35010.1 INTRACELLULAR A-AMYLASE | 0.0119 | 21.07 |
| | | [STREPTOCOCCUS MUTANS] | | |
| Strain 1-116 | SEQ ID NO: 132, | >CAZ78902.1 UNNAMED PROTEIN PRODUCT | 0.0009 | 21.80 |
| | SEQ ID NO: 204 | [THERMOACTINOMYCES VULGARIS] | | |
| Strain 1-117 | SEQ ID NO: 133 | >CAL15438.1 UNNAMED PROTEIN PRODUCT | 0.2743 | 26.67 |
| | | [ASPERGILLUS NIGER] | | |
| Strain 1-118 | SEQ ID NO: 134 | >AEC23345.1 PUTATIVE ALPHA-AMYLASE | 0.0029 | 21.48 |
| | | GLYCOSIDE HYDROLASE FAMILY 57, PARTIAL | | |
| | | [UNCULTURED BACTERIUM] | | |
| Strain 1-119 | SEQ ID NO: 135, | >AAK41420.1 CONSERVED HYPOTHETICAL | 0.0020 | 21.16 |
| | SEQ ID NO: 205 | PROTEIN [SULFOLOBUS SOLFATARICUS P2] | | |
| Strain 1-120 | SEQ ID NO: 136 | >AGW27504.1 ALPHA AMYLASE, PARTIAL | 0.0025 | 21.21 |
| | | [TRIBOLIUM CASTANEUM] | | |
| Strain 1-121 | SEQ ID NO: 137 | >CAB36742.1 ALPHA-AMYLASE-LIKE PROTEIN | 0.0032 | 21.40 |
| | | [ARABIDOPSIS THALIANA] | | |
| Strain 1-122 | SEQ ID NO: 138 | >AAA86835.1 ALPHA-AMYLASE PRECURSOR | 0.0797 | 17.29 |
| | | [PSEUDOMONAS SP. KFCC10818] | | |
| Strain 1-123 | SEQ ID NO: 139, | >AAA23005.1 ALPHA-AMYLASE [BUTYRIVIBRIO | 0.3475 | 22.69 |
| | SEQ ID NO: 206 | FIBRISOLVENS] | | |
| Strain 1-124 | SEQ ID NO: 140, | >BAN63122.1 ALPHA-AMYLASE [BACILLUS SP. | 0.0049 | 21.68 |
| | SEQ ID NO: 207 | AAH-31] | | |
| Strain 1-125 | SEQ ID NO: 141, | >AAQ01675.1 ALPHA-AMYLASE [ALKALIPHILIC | 0.0041 | 21.80 |
| | SEQ ID NO: 208 | BACTERIUM N10] | | |
| Strain 1-126 | SEQ ID NO: 142, | >AAC45781.1 ALPHA-AMYLASE | n.d. | n.d. |
| | SEQ ID NO: 209 | [LACTOBACILLUS AMYLOVORUS] | | |
| Strain 1-127 | SEQ ID NO: 143 | >ABX83871.1 ALPHA AMYLASE, PARTIAL | 0.0041 | 21.19 |
| | | [GEOBACILLUS THERMODENITRIFICANS] | | |
| Strain 1-128 | SEQ ID NO: 144, | >ADK21254.1 AMYLASE [UNCULTURED | 0.0023 | 20.87 |
| | SEQ ID NO: 210 | BACTERIUM] | | |
| Strain 1-129 | SEQ ID NO: 145 | >AAA26697.1 ALPHA-AMYLASE [STREPTOMYCES | 0.0045 | 20.76 |
| | | THERMOVIOLACEUS] | | |
| Strain 1-130 | SEQ ID NO: 146 | >AAA32935.1 ALPHA-AMYLASE [HORDEUM | 0.0063 | 21.65 |
| | | VULGARE] | | |
| Strain 1-131 | SEQ ID NO: 147 | >CAD62442.2 ALPHA-AMYLASE HOMOLOG MDE5 | 0.0062 | 22.21 |
| | | [SCHIZOSACCHAROMYCES POMBE] | | |
| Strain 1-132 | SEQ ID NO: 148 | >AAL37183.1 ALPHA AMYLASE A [CRASSOSTREA | 0.0799 | 22.50 |
| | | GIGAS] | | |
| Strain 1-133 | SEQ ID NO: 149, | >CAA49759.1 AMY [STREPTOMYCES LIVIDANS | 0.0005 | 21.90 |
| | SEQ ID NO: 211 | TK24] | | |
| Strain 1-134 | SEQ ID NO: 150 | >AAO62080.1 ALPHA AMYLASE [XANTHOMONAS | 0.4296 | 19.39 |
| | | CAMPESTRIS] | | |
| Strain 1-135 | SEQ ID NO: 151 | >CAA31586.1 UNNAMED PROTEIN PRODUCT | 0.0031 | 20.03 |
| | | [DICTYOGLOMUS THERMOPHILUM H-6-12] | | |
| Strain 1-136 | SEQ ID NO: 152 | >NP_721927.1 ALPHA-AMYLASE | 0.6526 | 20.27 |
| | | [STREPTOCOCCUS MUTANS UA159] | | |
| Strain 1-137 | SEQ ID NO: 153, | >AEM89278.1 PUTATIVE AMYLASE | 0.0039 | 20.82 |
| | SEQ ID NO: 212 | [UNCULTURED BACTERIUM] | | |
| Strain 1-138 | SEQ ID NO: 154, | >BAM75337.1 ALPHA-AMYLASE FAMILY | 0.0015 | 21.31 |
| | SEQ ID NO: 213 | STARCH-CONVERTING ENZYME [HALOARCULA | | |
| | | JAPONICA] | | |
| Strain 1-139 | SEQ ID NO: 155 | >AAV01045.1 SEQUENCE 2 FROM PATENT | 0.3792 | 22.10 |
| | | U.S. Pat No. 6,743,616 | | |
| Strain 1-140 | SEQ ID NO: 156 | >AGT20491.1 ALPHA-AMYLASE AMYSTU | 0.9975 | 21.26 |
| | | [ALTEROMONAS MACLEODII] | | |
| Strain 1-141 | SEQ ID NO: 157, | >ABS94974.1 Aspergillus kawachii | 0.0005 | 23.34 |
| | SEQ ID NO: 214 | | | |
| Strain 1-142 | SEQ ID NO: 158, | >OJJ55916 Aspergillus sydowii | 0.0041 | 21.09 |
| | SEQ ID NO: 215 | | | |
| Strain 1-143 | SEQ ID NO: 159, | >KUM56060 Penicillium freii | 0.0040 | 20.13 |
| | SEQ ID NO: 216 | | | |
| Strain 1-144 | SEQ ID NO: 160, | >KOS42177 Penicillium nordicum | 0.0039 | 4.14 |
| | SEQ ID NO: 217 | | | |
| Strain 1-145 | SEQ ID NO: 161 | >ADX42122 Aspergillus niger | 0.0026 | 20.29 |
| Strain 1-146 | SEQ ID NO: 162, | >XP_014174648 Grosmannia clavigera kw1407 | 0.0031 | 21.57 |
| | SEQ ID NO: 218 | | | |
| Strain 1-147 | SEQ ID NO: 163 | >KKK22656 Aspergillus rambellii | 0.0056 | 21.75 |
| Strain 1-148 | SEQ ID NO: 164 | >2GUY_A Aspergillus niger | −0.0003 | 22.55 |
| Strain 1-149 | SEQ ID NO: 165 | >ABL09312.1 Acarus siro | 0.0005 | 22.12 |
| Strain 1-150 | SEQ ID NO: 166 | >BAA86909.1 Apis mellifera | 0.0706 | 21.18 |
| Strain 1-151 | SEQ ID NO: 167, | >BAD06005.1 Aspergillus awamori | 0.0029 | 20.71 |
| | SEQ ID NO: 219 | | | |
| Strain 1-152 | SEQ ID NO: 168 | >OXN29793.1 Aspergillus fumigatus | 0.0000 | 20.49 |
| Strain 1-153 | SEQ ID NO: 169, | >OOO15193.1 Aspergillus oryzae | 0.0034 | 20.81 |
| | SEQ ID NO: 220 | | | |
| Strain 1-154 | SEQ ID NO: 170, | >ADH93703.1 Bacillus amyloliquefaciens | 0.3966 | 23.37 |
| | SEQ ID NO: 221 | | | |
| Strain 1-155 | SEQ ID NO: 171 | >P06278 Bacillus licheniformis | 0.0483 | 21.70 |
| Strain 1-156 | SEQ ID NO: 172, | >L8B068 Haloarcula japonica | −0.0009 | 22.50 |
| | SEQ ID NO: 222 | | | |
| Strain 1-157 | SEQ ID NO: 173 | >D4P4Y7 Saccharomycopsis fibuligera | 0.0519 | 21.83 |
| Strain 1-158 | SEQ ID NO: 174 | >P08144 Drosophila melanogaster | 0.1033 | 22.24 |
| Strain 1-159 | SEQ ID NO: 175, | >CBK65614.1 Bacteriodes xylanisolvens XB1A | 0.0020 | 20.77 |
| | SEQ ID NO: 223 | | | |
| Strain 1-160 | SEQ ID NO: 176 | >WP_055228734.1 Bacteriodes thetaiotamicron | 0.0007 | 20.91 |
| Strain 1-161 | SEQ ID NO: 177, | >WP_005849630.1 Bacteriodes vuglatus | 0.0991 | 20.95 |
| | SEQ ID NO: 224 | | | |
| Strain 1-162 | SEQ ID NO: 178, | >WP_007657674.1 Parabacteriodes goldsteinii | 0.0348 | 21.61 |
| | SEQ ID NO: 225 | | | |
| Strain 1-163 | SEQ ID NO: 179 | >AAB22383.2 Schwanniomyces occidentalis | 0.0647 | 25.03 |
| Strain 1-164 | SEQ ID NO: 180, | WP_044938644.1 Blautia schinkii | 0.2092 | 22.29 |
| | SEQ ID NO: 226 | | | |
| Strain 1-165 | SEQ ID NO: 181, | WP_089869030.1 Lachnospiraceae bacterium | −0.0054 | 21.37 |
| | SEQ ID NO: 227 | | | |
| Strain 1-166 | SEQ ID NO: 182, | WP_089869030.1 Lachnospiraceae bacterium G11] | 0.1870 | 20.70 |
| | SEQ ID NO: 228 | | | |
| Strain 1-167 | SEQ ID NO: 183, | WP_044947041.1 Blautia schinkii | 0.0030 | 20.59 |
| | SEQ ID NO: 229 | | | |
| Strain 1-168 | SEQ ID NO: 184, | WP_044946534.1 Blautia schinkii | 0.0275 | 20.36 |
| | SEQ ID NO: 230 | | | |
| Strain 1-169 | SEQ ID NO: 185, | WP_089864016.1 Lachnospiraceae bacterium G11 | 0.0053 | 20.43 |
| | SEQ ID NO: 231 | | | |
| Strain 1-170 | SEQ ID NO: 186 | WP_089868288.1 Lachnospiraceae bacterium G11 | 0.1189 | 22.22 |
| Strain 1-171 | SEQ ID NO: 187, | WP_044946435.1 Blautia schinkii | 0.0059 | 22.42 |
| | SEQ ID NO: 232 | | | |
| Strain 1-172 | SEQ ID NO: 188 | WP_049975157.1 Blautia schinkii | −0.0002 | 21.51 |
| Strain 1-173 | SEQ ID NO: 189, | WP_074756684.1 Butyrivibrio fibrisolvens | 0.0304 | 21.45 |
| | SEQ ID NO: 233 | | | |
| Strain 1-174 | SEQ ID NO: 190 | WP_027215175.1 Butyrivibrio fibrisolvens | 0.0025 | 20.80 |
| Strain 1-175 | SEQ ID NO: 191 | WP_089868286.1 Lachnospiraceae bacterium G11 | 0.0029 | 20.23 |

### Working Example #8. Transformation of Pullulanases in Strain 4

Strain 4 (CEN.PK 113-7D MATa wild type) was transformed with SEQ ID NO: 234. SEQ ID NO: 234 contains i) GPD1 promoter, ii) a truncated pullulanase from Klebsiella pneumoniae, iii) a CYC1 terminator, iv) a ScURA3 expression cassette, v) 2 micron origin of replication, and vi) a beta-lactamase expression cassette. Transformants were selected on synthetic dropout media lacking uracil (ScD-Ura). A representative strain from each was saved as Strain 4-1.

### Working Example #9. Pullulanase Activity in Strains Expressing Truncated Pullulanases from Klebsiella pneumoniae

Strain 4 and Strain 4-1 were grown aerobically for 24 hours at 30° C. in 50 mls of ScD-Ura media (6.7 g/L yeast nitrogen base, 1.9 g/L ScD-Ura drop out mix, 150 g/L glucose) contained in a 250 mL baffled shake flask. Cells were harvested by centrifugation at 4000 RPM for 5 minutes, washed with ice-cold water, and stored at −80° C. For the pullulanase assay, the pellets were thawed on ice, resuspended in 500 μL lysis buffer (50 mM Tris HCl buffer pH 6.8 containing 1% β-mercaptoethanol and 1× Halt™ Protease inhibitor cocktail) and transferred to mechanical lysis plate containing 500μ glass beads. Three bead beating cycles were carried out for lysis and cell lysate was collected after centrifuging at 14000 RPM for 10 minutes at 4.0° C. Pullulanase activity was determined using the protocol described above. Pullulanase activity for Strain 4.1 was 2.1 U/mg.

### Working Example #10. Construction of a Base Reporter Strain Expressing a Citrobacter braakii phyA-GFP Fusion

Strain 1 was transformed with SEQ ID NO: 235 and SEQ ID NO: 236. SEQ ID NO: 235 contains i) homology upstream of the FCY1 locus, ii) TDH3 promoter, iii) Citrobacter braakii phyA fused to Dasher-GFP, iv) CYC1 terminator, v) loxP recombination site, vi) URA3 promoter, and vii) partial URA3 gene. SEQ ID NO: 236 contains i) partial URA3 gene, ii) URA3 terminator, iii) homology downstream of the FCY1 locus. Transformants were selected on ScD-Ura agar plates. Resulting transformants are streaked to similar media for single colony isolation. Correct integration of SEQ ID NO: 235 and SEQ ID NO: 236 is verified by PCR. A PCR verified isolate was designated Strain 5. Strain 5 was transformed with SEQ ID NO: 237 and SEQ ID NO: 238. SEQ ID NO: 237 contains i) homology upstream of the FCY1 locus, ii) TDH3 promoter, iii) Citrobacter braakii phyA fused to Dasher-GFP, iv) CYC1 terminator, v) loxP recombination site, vi) TEF1 promoter, and vii) partial amdS gene from Aspergillus nidulans. SEQ ID NO: 238 contains i) partial amdS gene, ii) TEF1 terminator, iii) homology downstream of the FCY1 locus. Correct integration of SEQ ID NO: 237 and SEQ ID NO: 238 was verified by PCR. A PCR verified isolate was designated Strain 6.

### Working Example #11. Identifying AZC Concentration that Inhibits Growth in Saccharomyces cerevisiae

Ethanol Red™ was inoculated from a freshly grown YPD plate into 50 mL YPD (20 g/L yeast extract, 10 g/L yeast peptone, 20 g/L glucose) contained in a 250 mL baffled shake flask. The flask was incubated for 16 hours at 30° C. and 250 RPM. From this flask, the culture was diluted in 200 μL of YPD media having varying levels of AZC between 0-30 mM (azetidine-2-carboxylic acid), contained in a deep 96 well microtiter plate. The plate is incubated at 42° C. for 24 hours with shaking in a Molecular Devices Spectrophotometer, reading OD₆₀₀ every five minutes. The data shown in FIG. 1 shows the AZC dependent growth effect on Ethanol Red™.

### Working Example #12. 1^{st} Round of Mutagenesis and Selection with Strain 6 in Media Containing 20 mM AZC

Strain 6 was struck to a YPD plate and incubated at 30° C. until single colonies are visible (1-2 days). A slurry of OD₆₀₀ of 4 was made in Butterfields buffer and subjected to EMS mutagenesis. Following recovery, cells were transferred to a flask containing 50 mls of YPD in a 250 mL baffled shake flask and 20 mM AZC and incubated at 37° C. and 250 RPM. After 48 hours, the cells were centrifuged at 4000 RPM for 5 minutes and resuspended in 50 mL fresh YPD in a 250 mL baffled shake flask without AZC and grown for 16 hours at 37° C. and 250 RPM. The cell culture was harvested by centrifugation at 4000 RPM for 5 minutes and re-suspended in 10 mL Butterfields buffer prior to FACS analysis (A-pool).

### Working Example #13. Isolation of High Expressing CbphyA-Dasher Mutants from A-Pool Using FACS

Cells were sorted based on fluorescence using a BD FACSAria II flow cytometer. Gates were selected such that only the top 10% highest fluorescing cells were collected. The collected cells were centrifuged at 4000 RPM for 5 minutes and resuspended in 50 mL YPD and added to a 250 mL baffled shake flask. The flask was incubated at 30° C. for 16 hours. The cell culture if harvested by centrifugation at 4000 RPM, and frozen in 1 mL 15% glycerol at −80° C. (B-pool).

### Working Example #14. Screening High Expressing CbphyA-Dasher Mutants

From the frozen glycerol stock in Working Example 13 (B-pool), cells were diluted in water and plated on YPD to produce single colony isolates. Isolates were inoculated into 200 μL of PBS buffer in a 96 well plate to create a working stock. From this plate, 1.5 μL was used to inoculate a fresh deep well plate containing 300 μL of buffered ScD media (6.7 g/L yeast nitrogen base, 1.9 g/L ScD-Ura dropout mix, 20 g/L glucose with 0.1M MES pH 6.0) and grown for 24 hours at 30° C. and 1000 RPM. OD₆₀₀ was determined by diluting the culture 1:10 in water in a standard 96 well clear plate. RFU (relative fluorescent units) was determined by transferring 150 μL into a black 96 well plate and reading excitation/emission at 485/520 nm. Strain 6 and Ethanol Red™ were included as controls. RFU per OD₆₀₀ was compared to controls for each isolate, two of which are saved as Strain 6-1 and Strain 6-2. These strains show a 1.7 and 1.8-fold increase in RFU/OD compared to Strain 6.

### Working Example #15. Simultaneous Saccharification and Fermentation with Strains Having Improved RFU/OD

Strains 6, 6-1 and 6-2 were struck to a ScD-Ura plate and incubated at 30° C. until single colonies were visible (1-2 days). Cells from the ScD-Ura plate were scraped into sterile shake flask medium and the optical density (OD₆₀₀) is measured. Optical density was measured at wavelength of 600 nm with a 1 cm path length using a model Genesys20 spectrophotometer (Thermo Scientific). A shake flask was inoculated with the cell slurry to reach an initial OD600 of 0.1-0.3. Immediately prior to inoculating, 50 mL of shake flask medium was added to a 250 mL non-baffled shake flask (Corning 4995-250) fitted with a screw cap containing a gas-permeable seal (coming 1395-45LTMC). The shake flask medium includes of 725 g partially hydrolyzed corn starch, 150 g filtered light steep water, 50 g water, 25 g glucose, and 1 g urea. In addition, 25 μL of glucoamylase (Distillase, Dupont) was added to each flask. Duplicate flasks for each strain were incubated at 30° C. and 80% humidity with shaking in an orbital shaker at 100 rpm for 72 hours. Samples were taken at various intervals and analyzed for ethanol, growth, and RFU.

Table 3 shows the OD₆₀₀, RFU, RFU/OD₆₀₀, normalized RFU, and ethanol titers. Strain 6-1 and Strain 6-2 show higher RFU/OD ratios compared to Strain 6 throughout most of the experiment.

TABLE 3
Data from simultaneous saccharification and fermentation
with high expressing FACS sorted mutants

| | 0 h | 18 h | 24 h | 43 h | 72 h |
|---|---|---|---|---|---|
| OD600 | | | | | |
| Strain 6 | n.d. | 8.0 | 9.7 | 10.2 | 5.5 |
| Strain 6-1 | n.d. | 5.4 | 7.8 | 9.7 | 8.8 |
| Strain 6-2 | n.d. | 4.9 | 8.5 | 8.3 | 8.2 |
| RFU | | | | | |
| Strain 6 | n.d. | 1348.8 | 1070.6 | 952.2 | 336.6 |
| Strain 6-1 | n.d. | 1167.2 | 1607.4 | 1187.4 | 476.1 |
| Strain 6-2 | n.d. | 1166.0 | 1534.9 | 1025.6 | 408.8 |
| RFU/OD | | | | | |
| Strain 6 | n.d. | 169.0 | 110.3 | 93.6 | 61.3 |
| Strain 6-1 | n.d. | 218.2 | 206.3 | 122.9 | 54.3 |
| Strain 6-2 | n.d. | 238.4 | 179.7 | 123.0 | 50.2 |
| Normalized | | | | | |
| RFU/OD | | | | | |
| Strain 6 | n.d. | 1 | 1 | 1 | 1 |
| Strain 6-1 | n.d. | 1.3 | 1.9 | 1.3 | 0.9 |
| Strain 6-2 | n.d. | 1.4 | 1.6 | 1.3 | 0.8 |
| Ethanol (g/L) | | | | | |
| Strain 6 | 0.0 | 65.7 | 90.6 | 122.2 | 130.1 |
| Strain 6-1 | 0.0 | 57.1 | 85.0 | 132.3 | 136.2 |
| Strain 6-2 | 0.0 | 55.4 | 84.5 | 128.8 | 134.7 |

### Working Example #16. 2^{nd} Round of Mutagenesis and Selection with Previously Sorted Cells in Media Containing 20 mM AZC

Cells from the first round of FACS (B-pool) were struck to a YPD plate and incubated at 30° C. until single colonies were visible (1-2 days). A slurry of OD₆₀₀ of 4 was made in Butterfields buffer and subjected to EMS mutagenesis. Following recovery, cells were transferred to a flask containing 50 mls of YPD in a 250 mL baffled shake flask and 20 mM AZC and incubated at 38° C. and 250 RPM. After 48 hours, the cells were centrifuged at 4000 RPM for 5 minutes and resuspended in 50 mL fresh YPD in a 250 mL baffled shake flask without AZC and grown for 16 hours at 38° C. and 250 RPM. The cell culture was harvested by centrifugation at 4000 RPM for 5 minutes and re-suspended in 10 mL Butterfields buffer prior to FACS analysis (C-pool)

### Working Example #17. Isolation of High Expressing CbphyA-Dasher 2^{nd} Round Mutants from C-Pool Using FACS

Cells were sorted based on fluorescence using a BD FACSAria II flow cytometer. Gates were selected such that only the top 10% highest fluorescing cells were collected. The collected cells were centrifuged at 4000 RPM for 5 minutes and resuspended in 50 mL YPD and added to a 250 mL baffled shake flask. The flask was incubated at 30° C. for 16 hours. The cell culture was harvested by centrifugation at 4000 RPM, and frozen in 1 mL 15% glycerol at −80° C. (D-pool).

### Working Example #18. 3^{rd} Round of Mutagenesis and Selection with Previously Sorted Cells in Media Containing 20 mM AZC

Cells from the second round of FACS (D-pool) were struck to a YPD plate and incubated at 30° C. until single colonies are visible (1-2 days). A slurry of OD₆₀₀ of 4 was made in Butterfields buffer and subjected to EMS mutagenesis. Following recovery, cells were transferred to a flask containing 50 mls of YPD in a 250 mL baffled shake flask and 20 mM AZC and incubated at 39° C. and 250 RPM. After 48 hours, the cells were centrifuged at 4000 RPM for 5 minutes and resuspended in 50 mL fresh YPD in a 250 mL baffled shake flask without AZC and grown for 16 hours at 39° C. and 250 RPM. The cell culture was harvested by centrifugation at 4000 RPM for 5 minutes and re-suspended in 10 mL Butterfields buffer prior to FACS analysis (E-pool)

### Working Example #19. Isolation of High Expressing CbphyA-Dasher 3^{rd} Round Mutants from E-Pool Using FACS

Cells were sorted based on fluorescence using a BD FACSAria II flow cytometer. Gates were selected such that only the top 10% highest fluorescing cells were collected. The collected cells were centrifuged at 4000 RPM for 5 minutes and resuspended in 50 mL YPD and added to a 250 mL baffled shake flask. The flask was incubated at 30° C. for 16 hours. The cell culture was harvested by centrifugation at 4000 RPM, and frozen in 1 mL 15% glycerol at −80° C. (F-pool).

### Working Example #20. 4^{th} Round of Mutagenesis and Selection with Previously Sorted Cells in Media Containing 20 mM AZC

Cells from the second round of FACS (F-pool) were struck to a YPD plate and incubated at 30° C. until single colonies were visible (1-2 days). A slurry of OD₆₀₀ of 4 was made in Butterfields buffer and subjected to EMS mutagenesis. Following recovery, cells were transferred to a flask containing 50 mls of YPD in a 250 mL baffled shake flask and 20 mM AZC and incubated at 40° C. and 250 RPM. After 48 hours, the cells were centrifuged at 4000 RPM for 5 minutes and resuspended in 50 mL fresh YPD in a 250 mL baffled shake flask without AZC and grown for 16 hours at 40° C. and 250 RPM. The cell culture was harvested by centrifugation at 4000 RPM for 5 minutes and re-suspended in 10 mL Butterfields buffer prior to FACS analysis (G-pool).

### Working Example #21. Stabilization of G-Pool Mutants by Repeated Shake Flask Cultivation

Cells from G-pool were struck to a YPD plate and incubated at 30° C. until single colonies were visible (1-2 days). A patch of cells was inoculated into 50 mL YPD in a 250 mL baffled shake flask and incubated at 33.3° C. and 250 RPM for 24 hours. A 1 mL aliquot from this culture was used to seed a second identical culture. The process was repeated three times. Cells from the fourth flask were sorted similarly as above and a new high expressing population was saved (H-pool).

### Working Example #22. Screening High Expressing CbphyA-Dasher Mutants from H-Pool in High Throughput Microtiter Assay

From the frozen glycerol stock in Working Example 19 (H-pool), cells were diluted in water and plated on YPD to produce single colony isolates. Isolates were picked directly into 150 μL YPD contained in a black 96-well microtiter plate and grown for 24 hours at 37° C. and 400 RPM. OD₆₀₀ was determined by diluting the culture 1:10 in water in a standard 96 well clear plate. RFU was determined by assaying the remaining volume for excitation/emission (485/520 nm). Strain 6 and Ethanol Red™ were included as controls. RFU per OD₆₀₀ was compared to controls for each isolate. One isolate was saved as Strain 6-3. Strain 6-3 had an RFU/OD 1.125-fold higher compared to Strain 6.

### Working Example #23. Confirmation of High Expressing CbphyA-GFP Mutants Using LC/MS

Strains 6, 6-1, 6-2, 6-3 and Ethanol Red were struck to a ScD-Ura plate and incubated at 30° C. until single colonies were visible (1-2 days). Cells from the ScD-Ura plate were scraped into sterile shake flask medium and the optical density (OD₆₀₀) is measured. Optical density was measured at wavelength of 600 nm with a 1 cm path length using a model Genesys20 spectrophotometer (Thermo Scientific). A shake flask was inoculated with the cell slurry to reach an initial OD600 of 0.1-0.3. Immediately prior to inoculating, 50 mL of shake flask medium was added to a 250 mL non-baffled shake flask (Corning 4995-250) fitted with a screw cap containing a gas-permeable seal (coming 1395-45LTMC). The shake flask medium including 725 g partially hydrolyzed corn starch, 150 g filtered light steep water, 50 g water, 25 g glucose, and 1 g urea. In addition, 25 μL of glucoamylase (Distillase, Dupont) was added to each flask. Duplicate flasks for each strain were incubated at 30° C. and 80% humidity with shaking in an orbital shaker at 100 RPM for 72 hours. Samples were taken at various intervals and frozen at −20° C. for ethanol analysis or centrifuged at 4000 RPM for 5 minutes, washed with ice-cold water, centrifuged again at 4000 RPM for 5 minutes, and frozen at −80° C. for proteomics analysis. Ethanol and glucose concentrations were determined by high performance liquid chromatography with a refractive index detector. Proteomic analysis was determined using the following method. Cell pellets were thawed on ice and resuspended in 500 μL of 100 mM Tris HCl buffer pH 8. The suspension was then transferred to the 2 mL screw cap vial containing an equivalent volume of glass beads (500μ, Sigma G8772). Three 1 minute cell disruption cycles were carried out for mechanical lysis in the cold room, followed by centrifugation at 4° C., 14000 rpm for 10 minutes. The supernatant was transferred to 1.5 mL microcentrifuge tubes and placed on ice. Protein quantification in the cell free lysate (soluble fraction) was carried out using the PIERCE®660 nm protein assay reagent (Thermo Scientific Product #22660). The absorbance was read using SpectraMax (Molecular Devices) plate reader at 660 nm and BSA standards were used for determining the protein concentration. For LC/MS analysis, 100 μg of protein was digested with 5 μg of trypsin (Pierce, Product #90058) in a total volume of 100 μL of 100 mM Tris-HCl pH 8.0 at 37° C. for 24 hours. A Citrobacter braakii protein standard was synthesized in vitro using a PURExpress kit (NEB Catalog E6800) using SEQ ID NO: 239 as a template. Trypsin digested samples were injected onto an HPLC column (Agilent AdvanceBio Peptide Mapping) using an Agilent 1290 Infinity II autosampler. The HPLC column was coupled to a Thermo Q Exactive Plus mass spectrometer set to collect Top 10 data-dependent MS/MS fragmentation data at 35,000 resolution. The Citrobacter braakii phytase concentration for each replicate was averaged and divided by the total digested protein to calculate the average % phytase for each strain.

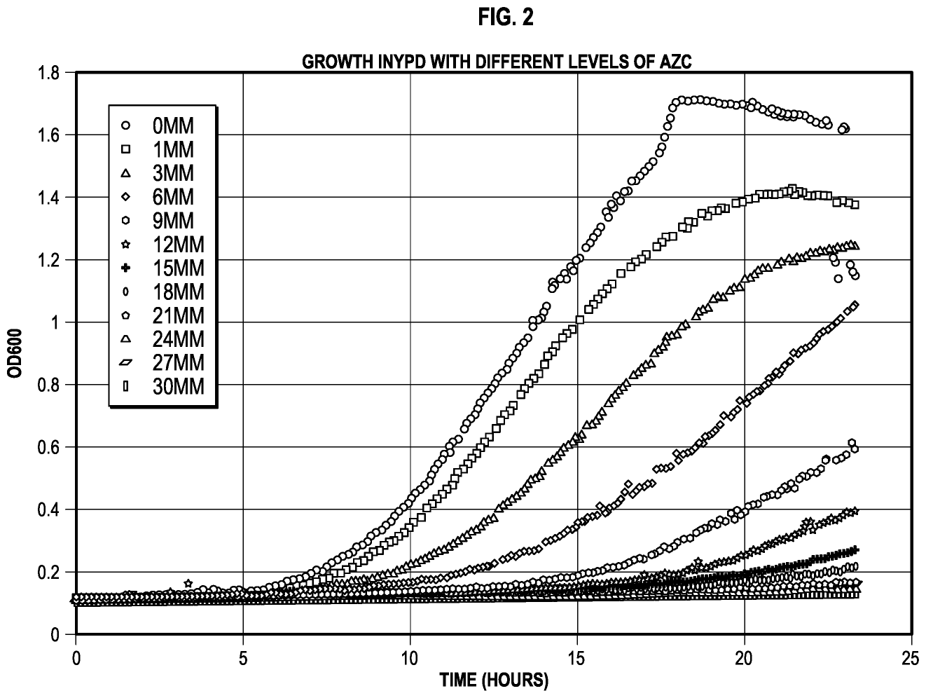

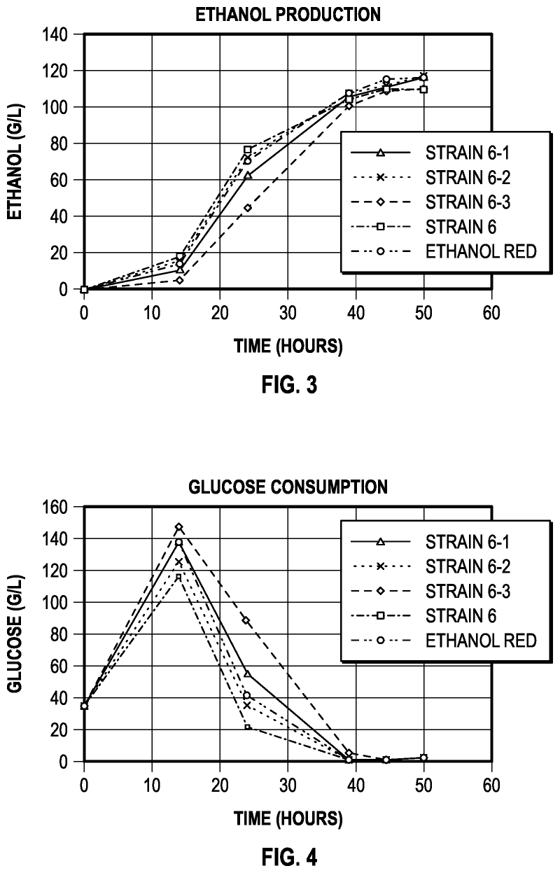

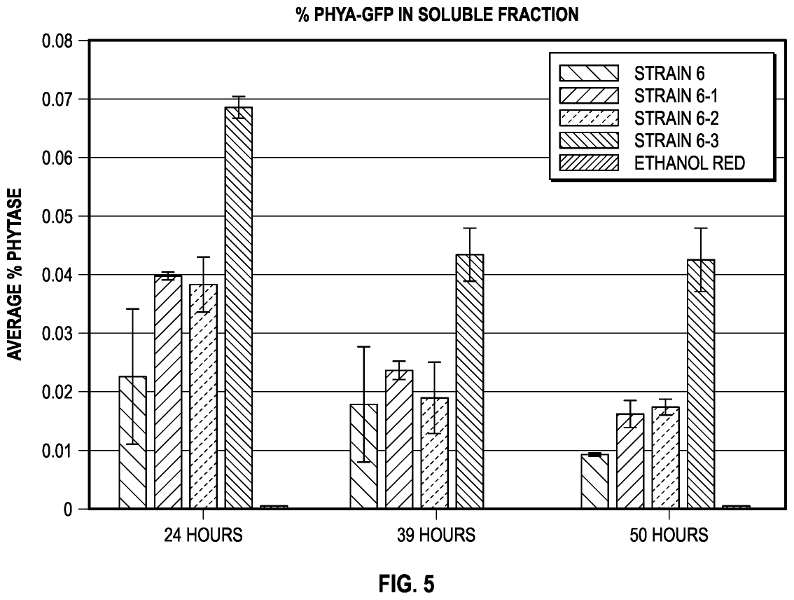

FIG. 2 shows the ethanol production profiles for the AZC+temperature mutant Strain 6-1, Strain 6-2 and Strain 6-3 relative to the mutagenesis and selection starting point Strain 6. Also included is the Wild Type control Ethanol Red™. At the end of fermentation (50 hours), Ethanol Red produces 115.8 g/L ethanol, whereas Strain 6 produces 109.5 g/L. The improved mutant Strain 6-1, Strain 6-2, and Strain 6-3 produce 116.1 g/L, 117.2 g/L, and 110.3 g/L respectively. FIG. 3 shows the glucose consumption profiles for the AZC+temperature mutant Strain 6-1, Strain 6-2 and Strain 6-3 relative to the mutagenesis and selection starting point Strain 6. At 50 hours the residual glucose for each of the strains is between 2.4-2.8 g/L. FIG. 4 shows the percent Citrobacter braakii phyA-GFP fusion as a function of the total intracellular protein (soluble fraction). At the 50 hour time point, Strain 6-1 shows a 75% improvement over Strain 6, Strain 6-2 shows a 83% improvement over Strain 6, and Strain 6-3 shows a 357% improvement in phyA-GFP concentration. FIG. 5 shows LC/MS data obtained.

### Working Example #24. Construction of Strains Harboring Shortlisted Phytases on CEN Plasmid

Strain 1 was transformed with SEQ ID NO 415 through SEQ ID NO 425. SEQ ID NO 415 through SEQ ID NO 425 contain i) a ScTDH3 promoter; ii) a codon optimized shortlisted phytase; iii) a ScCYC1 terminator; iv) a ScURA3 expression cassette; v) the Saccharomyces cerevisiae CEN6 centromere for stable replication; and vi) a beta-lactamase expression cassette. Transformants were selected on synthetic dropout media lacking uracil (ScD-Ura). Three representative strains from each transformation were saved as Strain 1-176 through Strain 1-208 and evaluated in ethanol fermentation studies.

### Working Example #25. Phytase Activity in Strains Harboring Shortlisted Phytases During Ethanol Fermentation

The wild type Ethanol Red strain and Strains 1-176 through 1-208 were struck to a ScD-Ura plate and incubated at 30° C. until single colonies were visible (1-2 days). Cells from the ScD-Ura plate were scraped into sterile shake flask medium containing 50 mL of shake flask medium targeting an initial OD600 of 0.2. The media was contained in a 250 mL non-baffled shake flask (Corning 4995-250) fitted with a screw cap containing a gas-permeable seal (coming 1395-45LTMC). The shake flask medium includes of 250 g Maltodextrin (Cargill Inc., Dayton Ohio), 6.7 g/L YNB w/o amino acids, and 1.9 g/L ScD-Ura amino acid mix. In addition, 20 μL of glucoamylase (Distillase, Dupont) was added to each flask. Singlet flasks for each strain were incubated at 30° C. and 80% humidity with shaking in an orbital shaker at 130 rpm for 67 hours. Two 1 mL samples were harvested 48 hours. The first was analyzed for ethanol by HPLC. The second was centrifuged at 10,000 RPM for 1 minute at 4° C. The supernatant was then removed (Broth), placed in a fresh tube and frozen at −80 C. The cell pellet was frozen at −80 C, then mechanically lysed similar as described above with the exception that no protease inhibitor cocktail added. Phytase activity was determined at pH 5.5 in both the Broth sample and the cell free extract (CFE) sample for each strain. The phytase assay was carried out for 44 minutes. The amount of phosphate released per minute in each assay was used to calculate the percentage of intracellular phytase activity for a sample (expressed as a percentage of total). To account for the differences in volume between the two samples for each strain (1 mL of broth and 0.5 mL of cell free extract, the cell free extract activity values are divided by 2). Where no detectable phosphate was detected, the minimum level of detection based on the potassium phosphate standard curve Pi was substituted (0.011 μmoles in 44 minutes). Where no detectable phosphate was detected in either sample, no value is given. The sample for Strain 1-185 was not determined (n.d).

Table 4 shows high levels of ethanol production for strains harbouring the shortlisted phytases, however only five retain activity under the conditions tested. At least 57% of the detectable phytase activity from Citrobacter amalonaticus is retained in the cells, whereas at least 13% of the detectable phytase from Citrobacter freundii is retained in the cells.

TABLE 4
Ethanol production and phytase activity in shake flasks with Maltodextrin as the sole carbon source.

| | | | 48 hr | Amount of Pi | Amount of Pi | | % |
|---|---|---|---|---|---|---|---|
| | | | EtOH | released in | released in | Limit of | intracellular |
| Strain ID | SEQ ID NO | Phytase present (protein sequence) | (g/L) | CFE (μmoles) | Broth (μmoles) | detection | Activity |
| Strain 1-176 | SEQ ID NO 415 | Citrobacter braakii (SEQ ID NO 326), | 61.4 | 0.000344 | 0.001638 | 0.000250 | 17% |
| Strain 1-177 | SEQ ID NO 415 | Citrobacter braakii (SEQ ID NO 326), | 62.9 | 0.000337 | 0.001432 | 0.000250 | 19% |
| Strain 1-178 | SEQ ID NO 415 | Citrobacter braakii (SEQ ID NO 326), | 63.1 | 0.000361 | 0.001505 | 0.000250 | 19% |
| Strain 1-179 | SEQ ID NO 416 | Citrobacter freundii (SEQ ID NO 267), | 63.6 | 0.000177 | 0.001137 | 0.000250 | 13% |
| Strain 1-180 | SEQ ID NO 416 | Citrobacter freundii (SEQ ID NO 267), | 65.6 | 0.000218 | 0.001119 | 0.000250 | 16% |
| Strain 1-181 | SEQ ID NO 416 | Citrobacter freundii (SEQ ID NO 267), | 65.1 | 0.000157 | 0.001034 | 0.000250 | 13% |
| Strain 1-182 | SEQ ID NO 417 | Citrobacter werkmanii (SEQ ID NO 268), | 64.3 | 0.000586 | 0.001144 | 0.000250 | 34% |
| Strain 1-183 | SEQ ID NO 417 | Citrobacter werkmanii (SEQ ID NO 268), | 68.0 | 0.000572 | 0.001108 | 0.000250 | 34% |
| Strain 1-184 | SEQ ID NO 417 | Citrobacter werkmanii (SEQ ID NO 268), | 65.9 | 0.000657 | 0.001092 | 0.000250 | 38% |
| Strain 1-185 | SEQ ID NO 418 | Citrobacter amalonaticus (SEQ ID NO 270), | 63.0 | n.d. | n.d. | 0.000250 | — |
| Strain 1-186 | SEQ ID NO 418 | Citrobacter amalonaticus (SEQ ID NO 270), | 61.6 | 0.000337 | b.d. | 0.000250 | 57% |
| Strain 1-187 | SEQ ID NO 418 | Citrobacter amalonaticus (SEQ ID NO 270), | 63.6 | 0.000393 | b.d. | 0.000250 | 61% |
| Strain 1-188 | SEQ ID NO 419 | Cronobacter sakazakii (SEQ ID NO 271), | 69.0 | b.d. | b.d. | 0.000250 | — |
| Strain 1-189 | SEQ ID NO 419 | Cronobacter sakazakii (SEQ ID NO 271), | 68.6 | b.d. | b.d. | 0.000250 | — |
| Strain 1-190 | SEQ ID NO 419 | Cronobacter sakazakii (SEQ ID NO 271), | 68.1 | b.d. | b.d. | 0.000250 | — |
| Strain 1-191 | SEQ ID NO 420 | Aspergillus japonicus (SEQ ID NO 311). | 72.5 | 0.000369 | 0.001461 | 0.000250 | 20% |
| Strain 1-192 | SEQ ID NO 420 | Aspergillus japonicus (SEQ ID NO 311). | 71.0 | 0.000438 | 0.001605 | 0.000250 | 21% |
| Strain 1-193 | SEQ ID NO 420 | Aspergillus japonicus (SEQ ID NO 311). | 70.1 | 0.000592 | 0.001590 | 0.000250 | 27% |
| Strain 1-194 | SEQ ID NO 421 | Azorhizobium caulinodans (SEQ ID NO | 68.0 | b.d. | b.d. | 0.000250 | — |
| | | 273), | | | | | |
| Strain 1-195 | SEQ ID NO 421 | Azorhizobium caulinodans (SEQ ID NO | 70.4 | b.d. | b.d. | 0.000250 | — |
| | | 273), | | | | | |
| Strain 1-196 | SEQ ID NO 421 | Azorhizobium caulinodans (SEQ ID NO | 70.8 | b.d. | b.d. | 0.000250 | — |
| | | 273), | | | | | |
| Strain 1-197 | SEQ ID NO 422 | Thermomyces lanuginosus(SEQ ID NO | 71.9 | b.d. | b.d. | 0.000250 | — |
| | | 244), | | | | | |
| Strain 1-198 | SEQ ID NO 422 | Thermomyces lanuginosus(SEQ ID NO | 71.4 | b.d. | b.d. | 0.000250 | — |
| | | 244), | | | | | |
| Strain 1-199 | SEQ ID NO 422 | Thermomyces lanuginosus(SEQ ID NO | 72.3 | b.d. | b.d. | 0.000250 | |
| | | 244), | | | | | |
| Strain 1-200 | SEQ ID NO 423 | Aspergillus awamori (SEQ ID NO 252), | 70.1 | 0.000625 | 0.002704 | 0.000250 | 19% |
| Strain 1-201 | SEQ ID NO 423 | Aspergillus awamori (SEQ ID NO 252), | 70.7 | 0.000697 | 0.002543 | 0.000250 | 22% |
| Strain 1-202 | SEQ ID NO 423 | Aspergillus awamori (SEQ ID NO 252), | 69.1 | 0.000612 | 0.002545 | 0.000250 | 19% |
| Strain 1-203 | SEQ ID NO 424 | Kosakonia sacchari (SEQ ID NO 269), | 71.6 | b.d. | b.d. | 0.000250 | — |
| Strain 1-204 | SEQ ID NO 424 | Kosakonia sacchari (SEQ ID NO 269), | 71.4 | b.d. | b.d. | 0.000250 | — |
| Strain 1-205 | SEQ ID NO 424 | Kosakonia sacchari (SEQ ID NO 269), | 69.2 | b.d. | b.d. | 0.000250 | — |
| Strain 1-206 | SEQ ID NO 425 | Peniophora lycii (SEQ ID NO 246), | 69.7 | b.d. | b.d. | 0.000250 | — |
| Strain 1-207 | SEQ ID NO 425 | Peniophora lycii (SEQ ID NO 246), | 72.5 | b.d. | b.d. | 0.000250 | — |
| Strain 1-208 | SEQ ID NO 425 | Peniophora lycii (SEQ ID NO 246), | 72.8 | b.d. | b.d. | 0.000250 | — |
| WT Ehanol | na | na | 83.3 | b.d. | b.d. | 0.000250 | — |
| Red | | | | | | | |

### Working Example #26. Evaluation of Shortlisted Phytases with N-Terminal Secretion Signals Removed in Shake Flasks

Strain 1 was transformed with SEQ ID NO 2: by itself or in combination with one of SEQ ID NO: 426 through SEQ ID NO: 436. SEQ ID NO: 2 contains i) an empty expression cassette containing the ScTDH3 promoter and ScCYC1 terminator; ii) a ScURA3 expression cassette; iii) the Saccharomyces cerevisiae CEN6 centromere for stable replication; and iv) a beta-lactamase expression cassette. SEQ ID NO: 426 through SEQ ID NO: 436 contain codon optimized shortlisted phytases that lack the predicted N-terminal secretion signal plus an additional 45 bp on the 5′ and 3′ ends that are homologous to the 5′ and 3′ ends on SEQ ID NO: 2 to facilitate shuttle vector cloning. Transformants were selected on synthetic dropout media lacking uracil (ScD-Ura). Three representative strains from each transformation were saved as Strain 1-209 through Strain 1-244 and evaluated in ethanol fermentation studies.

### Working Example #26. Evaluation of Shortlisted Phytases with N-Terminal Secretion Signals Removed in Shake Flasks

The wild type Ethanol Red strain and Strains 1-209 through 1-244 were struck to a ScD-Ura plate and incubated at 30° C. until single colonies were visible (1-2 days). Cells from the ScD-Ura plate were scraped into sterile shake flask medium containing 50 mL of shake flask medium targeting an initial OD600 of 0.2. The media was contained in a 250 mL non-baffled shake flask (Corning 4995-250) fitted with a screw cap containing a gas-permeable seal (coming 1395-45LTMC). The shake flask medium includes of 225 g Maltodextrin (Cargill Inc., Dayton Ohio), 6.7 g/L YNB w/o amino acids, and 1.9 g/L ScD-Ura amino acid mix. In addition, 20 μL of glucoamylase (Distillase, Dupont) was added to each flask. Singlet flasks for each strain were incubated at 30° C. and 80% humidity with shaking in an orbital shaker at 130 rpm for 48 hours. Two 1 mL samples were harvested 48 hours. The first was analyzed for ethanol by HPLC. The second was centrifuged at 10,000 RPM for 1 minute at 4° C. The supernatant was removed (Broth), placed in a fresh tube and frozen at −80 C. The cell pellet was frozen at −80 C, then mechanically lysed similar as described above with the exception that no protease inhibitor cocktail added. Phytase activity was determined at pH 5.5 in both the Broth samples and the Cell free extract samples for each strain. The phytase assay was carried out for 180 minutes. The amount of phosphate released per minute in each assay was used to calculate the percentage of intracellular phytase activity for a sample (expressed as a percentage of total). To account for the differences in volume between the two samples for each strain (1 mL of broth and 0.5 mL of cell free extract, the cell free extract activity values are divided by 2). Where no detectable phosphate was detected, the minimum level of detection based on the potassium phosphate standard curve Pi was substituted (0.5 mM, or 0.011 μmoles). Where no detectable phosphate was detected in either sample, no value is given.

Table 5 shows high levels of ethanol production while also maintaining high levels of encapsulated phytase for three of the shortlisted phytases. At least 83% of the detectable phytase activity from Citrobacter werkmanii is retained in the cells, whereas at least 44% of the detectable phytase from Kosakonia sacchari is retained in the cells. Phytase activity was not detectable in any of the broth samples.

TABLE 5
Ethanol production and phytase activity in shake flasks with Maltodextrin as the sole carbon source.

| | | | 48 hr | Amount of Pi | Amount of Pi | Limit of | % |
|---|---|---|---|---|---|---|---|
| | | | EtOH | released in | released in | detection | intracellular |
| Strain ID | SEQ ID NO | Phytase present | (g/L) | CFE (μmoles) | Broth (μmoles) | (μmoles) | Activity |
| Strain 1-209 | SEQ ID NO 426 | truncated Citrobacter braakii | 56.4 | b.d. | b.d. | 0.00006 | — |
| | | (SEQ ID NO 437) | | | | | |
| Strain 1-210 | SEQ ID NO 426 | truncated Citrobacter braakii | 58.6 | b.d. | b.d. | 0.00006 | — |
| | | (SEQ ID NO 437) | | | | | |
| Strain 1-211 | SEQ ID NO 426 | truncated Citrobacter braakii | 56.7 | b.d. | b.d. | 0.00006 | — |
| | | (SEQ ID NO 437) | | | | | |
| Strain 1-212 | SEQ ID NO 427 | truncated Citrobacter freundii | 57.4 | b.d. | b.d. | 0.00006 | — |
| | | (SEQ ID NO 438) | | | | | |
| Strain 1-213 | SEQ ID NO 427 | truncated Citrobacter freundii | 57.7 | b.d. | b.d. | 0.00006 | — |
| | | (SEQ ID NO 438) | | | | | |
| Strain 1-214 | SEQ ID NO 427 | truncated Citrobacter freundii | 58.8 | b.d. | b.d. | 0.00006 | — |
| | | (SEQ ID NO 438) | | | | | |
| Strain 1-215 | SEQ ID NO 428 | truncated Citrobacter werkmanii | 56.7 | 0.000298515 | b.d. | 0.00006 | 83% |
| | | (SEQ ID NO 439) | | | | | |
| Strain 1-216 | SEQ ID NO 428 | truncated Citrobacter werkmanii | 55.7 | 0.000285853 | b.d. | 0.00006 | 83% |
| | | (SEQ ID NO 439) | | | | | |
| Strain 1-217 | SEQ ID NO 428 | truncated Citrobacter werkmanii | 56.2 | 0.000333474 | b.d. | 0.00006 | 85% |
| | | (SEQ ID NO 439) | | | | | |
| Strain 1-218 | SEQ ID NO 429 | truncated Citrobacter amalonaticus | 52.7 | 0.000244931 | b.d. | 0.00006 | 80% |
| | | (SEQ ID NO 440) | | | | | |
| Strain 1-219 | SEQ ID NO 429 | truncated Citrobacter amalonaticus | 52.8 | 0.000284341 | b.d. | 0.00006 | 83% |
| | | (SEQ ID NO 440) | | | | | |
| Strain 1-220 | SEQ ID NO 429 | truncated Citrobacter amalonaticus | 52.2 | 0.000286199 | b.d. | 0.00006 | 83% |
| | | (SEQ ID NO 440) | | | | | |
| Strain 1-221 | SEQ ID NO 430 | truncated Cronobacter sakazakii | 58.9 | b.d. | b.d. | 0.00006 | — |
| | | (SEQ ID NO 441) | | | | | |
| Strain 1-222 | SEQ ID NO 430 | truncated Cronobacter sakazakii | 58.8 | b.d. | b.d. | 0.00006 | — |
| | | (SEQ ID NO 441) | | | | | |
| Strain 1-223 | SEQ ID NO 430 | truncated Cronobacter sakazakii | 58.5 | b.d. | b.d. | 0.00006 | — |
| | | (SEQ ID NO 441) | | | | | |
| Strain 1-224 | SEQ ID NO 431 | truncated Aspergillus japonicus | 56.7 | b.d. | b.d. | 0.00006 | — |
| | | (SEQ ID NO 442) | | | | | |
| Strain 1-225 | SEQ ID NO 431 | truncated Aspergillus japonicus | 55.9 | b.d. | b.d. | 0.00006 | — |
| | | (SEQ ID NO 442) | | | | | |
| Strain 1-226 | SEQ ID NO 431 | truncated Aspergillus japonicus | 58.0 | b.d. | b.d. | 0.00006 | — |
| | | (SEQ ID NO 442) | | | | | |
| Strain 1-227 | SEQ ID NO 432 | truncated Azorhizobium caulinodans | 58.0 | b.d. | b.d. | 0.00006 | — |
| | | (SEQ ID NO 443) | | | | | |
| Strain 1-228 | SEQ ID NO 432 | truncated Azorhizobium caulinodans | 58.3 | b.d. | b.d. | 0.00006 | — |
| | | (SEQ ID NO 443) | | | | | |
| Strain 1-229 | SEQ ID NO 432 | truncated Azorhizobium caulinodans | 58.5 | b.d. | b.d. | 0.00006 | — |
| | | (SEQ ID NO 443) | | | | | |
| Strain 1-230 | SEQ ID NO 433 | truncated Thermomyces | 58.1 | b.d. | b.d. | 0.00006 | — |
| | | lanuginosus (SEQ ID NO 444) | | | | | |
| Strain 1-231 | SEQ ID NO 433 | truncated Thermomyces | 59.6 | b.d. | b.d. | 0.00006 | — |
| | | lanuginosus (SEQ ID NO 444) | | | | | |
| Strain 1-232 | SEQ ID NO 433 | truncated Thermomyces | 57.9 | b.d. | b.d. | 0.00006 | — |
| | | lanuginosus (SEQ ID NO 444) | | | | | |
| Strain 1-233 | SEQ ID NO 434 | truncated Aspergillus awamori | 56.0 | b.d. | b.d. | 0.00006 | — |
| | | (SEQ ID NO 445) | | | | | |
| Strain 1-234 | SEQ ID NO 434 | truncated Aspergillus awamori | 55.8 | b.d. | b.d. | 0.00006 | — |
| | | (SEQ ID NO 445) | | | | | |
| Strain 1-235 | SEQ ID NO 434 | truncated Aspergillus awamori | 54.1 | b.d. | b.d. | 0.00006 | — |
| | | (SEQ ID NO 445) | | | | | |
| Strain 1-236 | SEQ ID NO 435 | truncated Kosakonia sacchari | 58.8 | 0.00004658 | b.d. | 0.00006 | 44% |
| | | (SEQ ID NO 446) | | | | | |
| Strain 1-237 | SEQ ID NO 435 | truncated Kosakonia sacchari | 59.3 | 0.00007817 | b.d. | 0.00006 | 57% |
| | | (SEQ ID NO 446) | | | | | |
| Strain 1-238 | SEQ ID NO 435 | truncated Kosakonia sacchari | 65.9 | b.d. | b.d. | 0.00006 | — |
| | | (SEQ ID NO 446) | | | | | |
| Strain 1-239 | SEQ ID NO 436 | truncated Peniophora lycii | 49.7 | b.d. | b.d. | 0.00006 | — |
| | | (SEQ ID NO 447) | | | | | |
| Strain 1-240 | SEQ ID NO 436 | truncated Peniophora lycii | 47.9 | b.d. | b.d. | 0.00006 | — |
| | | (SEQ ID NO 447) | | | | | |
| Strain 1-241 | SEQ ID NO 436 | truncated Peniophora lycii | 47.6 | b.d. | b.d. | 0.00006 | — |
| | | (SEQ ID NO 447) | | | | | |
| Strain 1-242 | SEQ ID NO 2 | Empty vector | 60.4 | b.d. | b.d. | 0.00006 | — |
| Strain 1-243 | SEQ ID NO 2 | Empty vector | 59.0 | b.d. | b.d. | 0.00006 | — |
| Strain 1-244 | SEQ ID NO 2 | Empty vector | 61.8 | b.d. | b.d. | 0.00006 | — |
| WT Ethanol | na | na | 65.7 | b.d. | b.d. | 0.00006 | — |
| Red | | | | | | | |

### Working Example #28. LC/MS Analysis to Confirm Phytase Encapsulation

To confirm the location of the truncated shortlisted phytases in both the CFE and Broth samples from the Working Example each was analyzed by LC/MS. To enable quantification, 11 tryptic peptides for each shortlisted phytase were directly synthesized (New England Peptides, USA). Prior to digestion, the 100 ul samples were reduced with the addition of 10 ul of a 500 mM solution of TCEP (Tris(2-carboxyethyl)phosphine hydrochloride) for 10 min at 60 C, and were then alkylated through the addition of 10 ul of a 200 mM of Indole Acetic Acid solution, incubated at room temperature for 30 minutes in the dark. Each sample was digested with a w/w ratio of 1 ug Trypsin (Pierce, Product 90058) to 20 ug of protein present at 37° C. for 24 hours. Post digestion, samples were subject to clean-up via Pierce Peptide Desalting Spin Columns (catalog #89851), before being lyophilized to dryness. Samples were then solubilized with 30 ul of a 95% H2O, 5% ACN, and 0.1% formic acid solution. Trypsin digested samples were injected onto an HPLC column (Acclaim Vanquish C18 Column, 250×2.1, 2.2 um part #0748125-V) using Thermo Vanquish autosampler. The HPLC column was coupled to a Thermo Fusion Lumos mass spectrometer set to MIPS (monoisotopic precursor selection) peptide mode at 120,000 resolution. A linear regression based on the standard curve for each shortlisted phytase was used to calculate the phytase concentration in the samples, as well as the limit of detection. To calculate the percent encapsulation for an individual phytase, the intracellular concentration was divided by the total. In samples that had no detectable phytase the limit of detection was substituted.

Table 6 shows seven out of the eleven truncated phytases tested were detectable in the intracellular cell lysate. Of those seven, five of the truncated phytases had on average greater than 50% encapsulation; truncated Citrobacter braakii, truncated Citrobacter freundii, truncated Thermomyces lanuginosus, truncated Kosakonia sacchari, and truncated Peniophora lycii.

TABLE 6
Quantification of intracellular and extracellular shortlisted phytases by LC/MS.

| | | Intracellular | Extracellular | Limit of | |
|---|---|---|---|---|---|
| | | concentration | concentration | detection | % |
| Strain ID | Phytase present | (μg/μL) | (μg/μL) | (μg/μL) | encapsulation |
| Strain 1-209 | truncated Citrobacter braakii (SEQ ID NO 437) | 0.000260335 | b.d. | 5.50432E−05 | 83% |
| Strain 1-210 | truncated Citrobacter braakii (SEQ ID NO 437) | 0.000145888 | b.d. | 5.50432E−05 | 73% |
| Strain 1-211 | truncated Citrobacter braakii (SEQ ID NO 437) | 6.15575E−05 | b.d. | 5.50432E−05 | 53% |
| Strain 1-212 | truncated Citrobacter freundii (SEQ ID NO 438) | 0.000146838 | b.d. | 7.50168E−05 | 66% |
| Strain 1-213 | truncated Citrobacter freundii (SEQ ID NO 438) | 0.000103891 | b.d. | 7.50168E−05 | 58% |
| Strain 1-214 | truncated Citrobacter freundii (SEQ ID NO 438) | 0.000148706 | b.d. | 7.50168E−05 | 66% |
| Strain 1-215 | truncated Citrobacter werkmanii (SEQ ID NO 439) | 2.82135E−06 | b.d. | 3.59193E−05 | — |
| Strain 1-216 | truncated Citrobacter werkmanii (SEQ ID NO 439) | 7.39526E−06 | b.d. | 3.59193E−05 | — |
| Strain 1-217 | truncated Citrobacter werkmanii (SEQ ID NO 439) | b.d. | b.d. | 3.59193E−05 | — |
| Strain 1-218 | truncated Citrobacter amalonaticus (SEQ ID NO 440) | 4.44993E−05 | 6.44515E−05 | 6.28994E−05 | 41% |
| Strain 1-219 | truncated Citrobacter amalonaticus (SEQ ID NO 440) | 4.3419E−05 | b.d. | 6.28994E−05 | 41% |
| Strain 1-220 | truncated Citrobacter amalonaticus (SEQ ID NO 440) | 4.63435E−05 | 6.91236E−05 | 6.28994E−05 | 40% |
| Strain 1-221 | truncated Cronobacter sakazakii (SEQ ID NO 441) | 2.57921E−05 | b.d. | 6.31503E−05 | — |
| Strain 1-222 | truncated Cronobacter sakazakii (SEQ ID NO 441) | b.d. | b.d. | 6.31503E−05 | — |
| Strain 1-223 | truncated Cronobacter sakazakii (SEQ ID NO 441) | b.d. | b.d. | 6.31503E−05 | — |
| Strain 1-224 | truncated Aspergillus japonicus (SEQ ID NO 442) | b.d. | b.d. | 0.000233906 | — |
| Strain 1-225 | truncated Aspergillus japonicus (SEQ ID NO 442) | b.d. | b.d. | 0.000233906 | — |
| Strain 1-226 | truncated Aspergillus japonicus (SEQ ID NO 442) | b.d. | b.d. | 0.000233906 | — |
| Strain 1-227 | truncated Azorhizobium caulinodans (SEQ ID NO 443) | 0.000108875 | 0.000202369 | 0.000194103 | 35% |
| Strain 1-228 | truncated Azorhizobium caulinodans (SEQ ID NO 443) | 0.000115044 | 0.00020055 | 0.000194103 | 36% |
| Strain 1-229 | truncated Azorhizobium caulinodans (SEQ ID NO 443) | 0.000123466 | b.d. | 0.000194103 | 39% |
| Strain 1-230 | truncated Thermomyces lanuginosus (SEQ ID NO 444) | 1.81079E−05 | b.d. | 1.67004E−05 | 52% |
| Strain 1-231 | truncated Thermomyces lanuginosus (SEQ ID NO 444) | 6.7351E−05 | b.d. | 1.67004E−05 | 80% |
| Strain 1-232 | truncated Thermomyces lanuginosus (SEQ ID NO 444) | 7.14828E−05 | b.d. | 1.67004E−05 | 81% |
| Strain 1-233 | truncated Aspergillus awamori (SEQ ID NO 445) | b.d. | b.d. | 0.000234455 | — |
| Strain 1-234 | truncated Aspergillus awamori (SEQ ID NO 445) | b.d. | b.d. | 0.000234455 | — |
| Strain 1-235 | truncated Aspergillus awamori (SEQ ID NO 445) | b.d. | b.d. | 0.000234455 | — |
| Strain 1-236 | truncated Kosakonia sacchari (SEQ ID NO 446) | 0.000121127 | b.d. | 4.83082E−05 | 71% |
| Strain 1-237 | truncated Kosakonia sacchari (SEQ ID NO 446) | 0.000260901 | b.d. | 4.83082E−05 | 84% |
| Strain 1-238 | truncated Kosakonia sacchari (SEQ ID NO 446) | 0.000131192 | b.d. | 4.83082E−05 | 73% |
| Strain 1-239 | truncated Peniophora lycii (SEQ ID NO 447) | 4.7883E−05 | b.d. | 4.11568E−05 | 54% |
| Strain 1-240 | truncated Peniophora lycii (SEQ ID NO 447) | 4.71217E−05 | b.d. | 4.11568E−05 | 53% |
| Strain 1-241 | truncated Peniophora lycii (SEQ ID NO 447) | 8.43785E−05 | 4.13682E−05 | 4.11568E−05 | 67% |

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the numbering of which is not to be construed as designating levels of importance:

Embodiment 1 provides a fermentation method, the method comprising:

producing at least about 10 g/L of a bioproduct and one or more heterologous polypeptides by fermenting a medium using an engineered microorganism, wherein about 2 wt % to about 100 wt % of the one or more heterologous polypeptides are encapsulated intercellularly in the engineered microorganism; and

isolating the engineered microorganism comprising the encapsulated one or more heterologous polypeptides, wherein about 50 wt % to about 100 wt % of the one or more heterologous polypeptides retain functionality following isolation of the engineered microorganism.

Embodiment 2 provides the fermentation method of Embodiment 1, wherein the engineered microorganism comprises a eukaryotic cell or a prokaryotic cell.

Embodiment 3 provides the fermentation method of any one of Embodiments 1 or 2, wherein the engineered microorganism comprises a bacteria or a fungus.

Embodiment 4 provides the fermentation method of any one of Embodiments 1-3, wherein the engineered microorganism comprises Streptococcus, Lactobacillus, Bacillus, Escherichia, Salmonella, Neisseria, Acetobactor, Arthrobacter, Aspergillus, Bifdobacterium, Corynebacterium, Pseudomonas fluorescens, or a mixture thereof.

Embodiment 5 provides the fermentation method of any one of Embodiments 1-4, wherein the engineered microorganism comprises Escherichia coli.

Embodiment 6 provides the fermentation method of any one of Embodiments 1-5, wherein the engineered microorganism comprises a yeast, a filamentous fungi, or a mixture thereof.

Embodiment 7 provides the fermentation method of Embodiment 6, wherein the yeast comprises Saccharomyces cerevisiae, Komagataella sp., Kluyveromyces lactis, Yarrowia lipolytica, Issatchenkia orientalis or a mixture thereof.

Embodiment 8 provides the fermentation method of any one of Embodiments 6 or 7 wherein the filamentous fungi comprises Aspergillus, Trichoderma, Myceliophthora thermophila, or a mixture thereof.

Embodiment 9 provides the fermentation method of any one of claims 1-8, wherein the engineered microorganism comprises an expression vector, an edited genome, or a combination thereof.

Embodiment 10 provides the fermentation method of Embodiment 9, wherein the vector comprises an integration plasmid, episomal plasmid, a centromeric plasmid, or a combination thereof.

Embodiment 11 provides the fermentation method of any one of Embodiments 1-10, wherein a greater amount of the one or more heterologous polypeptides are encapsulated intercellularly in the engineered microorganism than is secreted from the engineered microorganism during fermentation.

Embodiment 12 provides the fermentation method of any one of Embodiments 1-11, wherein the bioproduct comprises a (C₁-C₂₀)hydrocarbyl.

Embodiment 13 provides the fermentation method of any one of Embodiments 1-12, wherein the bioproduct comprises ethanol, an organic acid, or a combination thereof.

Embodiment 14 provides the fermentation method of any one of Embodiments 1-13, wherein the bioproduct comprises ethanol.

Embodiment 15 provides the fermentation method of any one of Embodiments 1-14, wherein at least 25 g/L bioproduct is produced.

Embodiment 16 provides the fermentation method of any one of Embodiments 1-15, wherein at least 50 g/L bioproduct is produced.

Embodiment 17 provides the fermentation method of any one of Embodiments 1-16, wherein about 10 g/L to about 200 g/L bioproduct is produced.

Embodiment 18 provides the fermentation method of any one of Embodiments 1-17, wherein about 20 g/L to about 80 g/L bioproduct is produced.

Embodiment 19 provides the fermentation method of any one of Embodiments 1-18, wherein the encapsulated one or more heterologous polypeptides are at least about 10% of a total cellular polypeptide level in the engineered microorganism.

Embodiment 20 provides the fermentation method of any one of Embodiments 1-19, wherein the encapsulated one or more heterologous polypeptides are at least about 30% of a total cellular polypeptide level in the engineered microorganism.

Embodiment 21 provides the fermentation method of any one of Embodiments 1-20, wherein the encapsulated one or more heterologous polypeptides are in a range of from about 5% to about 90% of a total cellular polypeptide level in the engineered microorganism.

Embodiment 22 provides the fermentation method of any one of Embodiments 1-21, wherein the encapsulated one or more heterologous polypeptides are in a range of from about 10% to about 70% of a total cellular polypeptide level in the engineered microorganism.

Embodiment 23 provides the fermentation method of any one of Embodiments 1-22, wherein the encapsulated one or more heterologous polypeptides are in a range of from about 15% to about 30% of a total cellular polypeptide level in the engineered microorganism.

Embodiment 24 provides the fermentation method of any one of Embodiments 1-23, wherein about 70 wt % to about 95 wt % of the of the one or more heterologous polypeptides retain functionality following isolation of the engineered microorganism.

Embodiment 25 provides the fermentation method of any one of Embodiments 1-24, wherein the functionality of the one or more enzymes comprises enzymatic activity.

Embodiment 26 provides the fermentation method of any one of Embodiments 1-25, wherein the encapsulated one or more heterologous polypeptides comprise one or more enzyme.

Embodiment 27 provides the fermentation method of any one of Embodiments 1-26, wherein the encapsulated one or more heterologous polypeptides comprise one or more enzymes chosen from oxidoreductases, transferases, hydrolases, lyases, isomerases, ligases, and translocases.

Embodiment 28 provides the fermentation method of any one of Embodiments 1-27, wherein the one or more enzymes comprise a transferase.

Embodiment 29 provides the fermentation method of Embodiment 28, wherein the transference is a glycosidase.

Embodiment 30 provides the fermentation method of any one of Embodiments 1-29, wherein the encapsulated one or more heterologous polypeptides comprise a phytase, an alpha-amylase, a glucoamylase, a glucanase, a lipase, an alkaline extracellular protease, an invertase, a galactanase I, an α-amylase, an α-galactosidase, a peroxidase, an aspartic proteinase II, or a mixture thereof.

Embodiment 31 provides the fermentation method of any one of Embodiments 1-30, further comprising distilling the bioproduct.

Embodiment 32 provides the fermentation method of Embodiment 31, wherein a majority of the one or more heterologous polypeptides further exhibit enzymatic activity following distillation of the bioproduct.

Embodiment 33 provides the fermentation method of Embodiment 32, wherein the enzymatic activity of the heterologous polypeptide is in a range of from about 0.1 U/mg to about 5 U/mg.

Embodiment 34 provides the fermentation method of any one of Embodiments 32 or 33, wherein the enzymatic activity of the heterologous polypeptide is in a range of from about 0.2 U/mg to about 1 U/mg.

Embodiment 35 provides the fermentation method of any one of Embodiments 1-34, wherein at least a portion of the one or more heterologous polypeptides are post-translationally modified.

Embodiment 36 provides the fermentation method of Embodiment 35, wherein the post-translation modification comprises acetylation, amidation, hydroxylation, methylation, N-linked glycosylation, O-linked glycosylation, phosphorylation, pyrrolidone carboxylic acid, sulfation, ubiquitylation, or a combination thereof.

Embodiment 37 provides the fermentation method of any one of Embodiments 1-36, further comprising screening a strain of the one or more heterologous polypeptides for enzymatic activity.

Embodiment 38 provides the fermentation method of Embodiment 37, wherein the one or more heterologous polypeptide is screened for intracellular enzymatic activity.

Embodiment 39 provides the fermentation method of any one of Embodiments 37 or 38, further comprising determining whether the one or more heterologous polypeptides meets or exceeds a threshold enzymatic activity value.

Embodiment 40 provides the fermentation method of Embodiment 39, wherein the encapsulated one or more heterologous polypeptides meets or exceeds the threshold enzymatic activity value.

Embodiment 41 provides the fermentation method of any one of Embodiments 1-40, further comprising screening a strain of the one or more heterologous polypeptides for the ability be substantially retained intercellularly.

Embodiment 42 provides the fermentation method of any one of Embodiments 1-41, wherein the medium comprises a carbohydrate.

Embodiment 43 provides the fermentation method of Embodiment 42, wherein the carbohydrate comprises a glucose, a glucose oligomer, or a mixture thereof.

Embodiment 44 provides the fermentation method of any one of Embodiments 1-43, wherein the one or more heterologous polypeptides are a first class of heterologous polypeptides and the method further produces a second class of heterologous polypeptides.

Embodiment 45 provides the fermentation method of Embodiment 44, wherein the first class of heterologous polypeptides and the second class of heterologous polypeptides are different.

Embodiment 46 provides the fermentation method of any one of Embodiments 44 or 45, wherein the second heterologous polypeptide class comprises a non-enzymatic heterologous polypeptide.

Embodiment 47 provides the fermentation method of Embodiment 46, wherein the non-enzymatic heterologous polypeptide comprises an al-antitrypsin, a hiruidin, a transferrin, an insulin, a serum albumin, collagen, an interferon-alpha 2b, or mixtures thereof.

Embodiment 48 provides the fermentation method of any one of Embodiments 1-47, wherein the engineered microorganism further produces a heterologous amino acid, a heterologous antigen, a heterologous cofactor, a heterologous hormone, a heterologous vitamin, a heterologous lipid, a heterologous pharmaceutical, or a mixture thereof.

Embodiment 49 provides the fermentation method of Embodiment 48, wherein the amino acid is an essential amino acid to an animal.

Embodiment 50 provides the fermentation method of Embodiment 49, wherein the essential amino acid comprises lysine, methionine, phenylalanine, threonine, isoleucine, tryptophan, valine, leucine, arginine, taurine, histidine, or mixtures thereof.

Embodiment 51 provides the fermentation method of any one of Embodiments 48-50, wherein the vitamin comprises vitamin A, vitamin B1, vitamin B2, vitamin B3, vitamin B5, vitamin B6, vitamin B7, vitamin B9, vitamin B12, vitamin C, vitamin D1, vitamin D2, vitamin D3, vitamin D4, a tocopherol, vitamin K, or mixtures thereof.

Embodiment 52 provides the fermentation method of any one of Embodiments 48-51, wherein the hormone comprises an insulin precursor, a glucagon, or a mixture thereof.

Embodiment 53 provides the fermentation method of any one of Embodiments 48-52, wherein the antigen comprises a hepatitis surface antigen.

Embodiment 54 provides the fermentation method of any one of Embodiments 1-53, further comprising mixing the isolated microorganism with a substrate.

Embodiment 55 provides the fermentation method of Embodiment 54, wherein the substrate comprises cellulose, a wood chip, a vegetable, a biomass, animal waste, oat, wheat, corn, barley, milo, millet, rice, rye, sorghum, potato, sugar beets, taro, cassaya, a fruit, a fruit juice, a sugar cane, or mixtures thereof.

Embodiment 56 provides the fermentation method of any one of Embodiments 54 or 55, wherein the substrate comprises animal feed.

Embodiment 57 provides the fermentation method of any one of Embodiments 1-56, further comprising mixing the bioproduct with gasoline.

Embodiment 58 provides the fermentation method of any one of Embodiments 1-57, further comprising isolating the one or more heterologous polypeptides from the engineered microorganism.

Embodiment 59 provides the fermentation method of Embodiment 58, wherein removing the one or more heterologous polypeptides comprises lysing the engineered microorganism, secreting the one or more heterologous polypeptides from the engineered microorganism, or both.

Embodiment 60 provides the fermentation method of any one of Embodiments 1-59, wherein the engineered microorganism secretes comparatively less of the one or more heterologous polypeptides than a corresponding naturally occurring microorganism.

Embodiment 61 provides the fermentation method of any one of Embodiments 1-60, wherein the fermentation method is run for a period of time in a range of from about 0.5 hours to about 40 hours.

Embodiment 62 provides the fermentation method of any one of Embodiments 1-61, wherein the fermentation method is run for a period of time in a range of from about 2 hours to about 10 hours.

Embodiment 63 provides the fermentation method of any one of Embodiments 1-62, wherein the engineered microorganism further comprises a nucleic acid encoding the one or more heterologous polypeptides.

Embodiment 64 provides the fermentation method of Embodiment 63, wherein the nucleic acid encoding the one or more heterologous polypeptides is part of a vector.

Embodiment 65 provides the fermentation method of any one of Embodiments 1-64, further comprising introducing an amino acid analogue to the engineered microorganism to result in overexpression of the heterologous polypeptide relative to an engineered microorganism that is free of the amino acid analogue.

Embodiment 66 provides the fermentation method of any one of Embodiments 1-65, wherein the amino acid analogue comprises azetidine-2-carboxylic acid.

Embodiment 67 provides a fermentation method, the method comprising:

producing at least about 10 g/L ethanol and one or more heterologous polypeptides by fermenting a medium using an engineered yeast comprising an expression vector, an edited genome, or a combination thereof, wherein about 20 wt % to about 100 wt % of the one or more heterologous polypeptides are encapsulated intercellularly in the engineered yeast; and

isolating the engineered yeast comprising the encapsulated one or more heterologous polypeptides, wherein the one or more heterologous polypeptides exhibit enzymatic activity following isolation of the engineered yeast.

Embodiment 68 provides an engineered microorganism adapted to ferment a medium and produce at least about 10 g/L of a bioproduct, the engineered microorganism comprising:

one or more exogenous polynucleotides encoding one or more heterologous polypeptides, wherein when expressed from the exogenous polynucleotide the heterologous polypeptide is encapsulated in the engineered microorganism, the one or more heterologous polypeptides comprising at least about 5% of a total cellular polypeptide level in the engineered microorganism, wherein about 50 wt % to about 100 wt % of the one or more heterologous polypeptides retain their functionality following isolation of the engineered microorganism.

Embodiment 69 provides the engineered microorganism of Embodiment 68, wherein about 50 wt % to about 100 wt % of the one or more heterologous polypeptides retain their functionality following a distillation process to which the engineered microorganism is exposed.

Embodiment 70 provides the engineered microorganism of any one of Embodiments 68 or 69, wherein the engineered microorganism comprises a eukaryotic cell or a prokaryotic cell.

Embodiment 71 provides the engineered microorganism of any one of Embodiments 68-70, wherein the engineered microorganism comprises a bacteria or a fungus.

Embodiment 72 provides the engineered microorganism of any one of Embodiments 68-71, wherein the engineered microorganism comprises Streptococcus, Lactobacillus, Bacillus, Escherichia, Salmonella, Neisseria, Acetobactor, Arthrobacter, Aspergillus, Bifdobacterium, Corynebacterium, Pseudomonas fluorescens, or a mixture thereof.

Embodiment 73 provides the engineered microorganism of any one of Embodiments 68-72, wherein the engineered microorganism comprises Escherichia coli.

Embodiment 74 provides the engineered microorganism of any one of Embodiments 68-73, wherein the engineered microorganism comprises a yeast, a filamentous fungi, or a mixture thereof.

Embodiment 75 provides the engineered microorganism of Embodiment 74, wherein the yeast comprises Saccharomyces cerevisiae, Komagataella sp., Kluyveromyces lactis, Yarrowia lipolytica, Issatchenkia orientalis, or a mixture thereof.

Embodiment 76 provides the engineered microorganism of any one of Embodiments 74 or 75 wherein the filamentous fungi comprises Aspergillus, Trichoderma, Myceliophthora thermophila, or a mixture thereof.

Embodiment 77 provides the engineered microorganism of any one of Embodiments 68-76, wherein the engineered microorganism comprises an expression vector, a genomically integrated polynucleotide sequence, or a combination thereof.

Embodiment 78 provides the engineered microorganism of Embodiment 77, wherein the vector comprises an integration plasmid, episomal plasmid, a centromeric plasmid, or a combination thereof.

Embodiment 79 provides the engineered microorganism of any one of Embodiments 68-78, wherein a greater amount of the one or more heterologous polypeptides are encapsulated intercellularly in the engineered microorganism than is secreted from the engineered microorganism during fermentation of the medium.

Embodiment 80 provides the engineered microorganism of any one of Embodiments 68-79, wherein the bioproduct comprises a (C₁-C₂₀)hydrocarbyl.

Embodiment 81 provides the engineered microorganism of any one of Embodiments 68-80, wherein bioproduct comprises ethanol, an organic acid, or a combination thereof.

Embodiment 82 provides the engineered microorganism of any one of Embodiments 68-81, wherein the engineered microorganism is adapted to produce a bioproduct during fermentation comprising ethanol.

Embodiment 83 provides the engineered microorganism of any one of Embodiments 68-82, wherein the engineered microorganism is adapted to produce at least 25 g/L bioproduct during fermentation.

Embodiment 84 provides the engineered microorganism of any one of Embodiments 68-83, wherein the engineered microorganism is adapted to produce at least 50 g/L bioproduct during fermentation.

Embodiment 85 provides the engineered microorganism of any one of Embodiments 68-84, wherein the engineered microorganism is adapted to produce about 10 g/L to about 200 g/L bioproduct during fermentation.

Embodiment 86 provides the engineered microorganism of any one of Embodiments 68-85, wherein the enzymatic activity of the heterologous polypeptide is in a range of from about 0.1 U/mg to about 5 U/mg.

Embodiment 87 provides the engineered microorganism of any one of Embodiments 68-86, wherein the enzymatic activity of the heterologous polypeptide is in a range of from about 0.2 U/mg to about 1 U/mg.

Embodiment 88 provides the engineered microorganism of any one of Embodiments 68-87, wherein the engineered microorganism is adapted to produce about 20 g/L to about 80 g/L bioproduct during fermentation.

Embodiment 89 provides the engineered microorganism of any one of Embodiments 68-88, wherein the encapsulated one or more heterologous polypeptides are at least about 30% of a total cellular polypeptide level in the engineered microorganism.

Embodiment 90 provides the engineered microorganism of any one of Embodiments 68-89, wherein the encapsulated one or more heterologous polypeptides are in a range of from about 5% to about 90% of a total cellular polypeptide level in the engineered microorganism.

Embodiment 91 provides the engineered microorganism of any one of Embodiments 68-90, wherein the encapsulated one or more heterologous polypeptides are in a range of from about 10% to about 70% of a total cellular polypeptide level in the engineered microorganism.

Embodiment 92 provides the engineered microorganism of any one of Embodiments 68-91, wherein about 70 wt % to about 95 wt % of the of the one or more heterologous polypeptides retain functionality following isolation of the engineered microorganism.

Embodiment 93 provides the engineered microorganism of any one of Embodiments 68-92, wherein the encapsulated one or more heterologous polypeptides comprise one or more enzymes chosen from oxidoreductases, transferases, hydrolases, lyases, isomerases, ligases, and translocases.

Embodiment 94 provides the engineered microorganism of any one of Embodiments 68-93, wherein the one or more enzymes comprise a transferase.

Embodiment 95 provides the engineered microorganism of any one of Embodiments 68-94, wherein the transference is a glycosidase.

Embodiment 96 provides the engineered microorganism of any one of Embodiments 68-95, wherein the encapsulated one or more heterologous polypeptides comprise a phytase, an alpha-amylase, a glucoamylase, a glucanase, a lipase, an alkaline extracellular protease, an erythropoietin, an invertase, a galactanase I, an α-amylase, an α-galactosidase, a peroxidase, an aspartic proteinase II, or mixtures thereof.

Embodiment 97 provides the engineered microorganism of any one of Embodiments 68-96, wherein the exogenous polynucleotide encodes a phytase (EC 3.1.3.8, 3.1.3.26, or 3.1.3.72) at least 80%, at least 85%, at least 90%, or at least 95% identical to at least one of SEQ ID NOs: 240-326 and 437-447.

Embodiment 98a provides the engineered microorganism of any one of Embodiments 68-97, wherein the exogenous polynucleotide encodes a phytase (EC 3.1.3.8, 3.1.3.26, or 3.1.3.72) at least 80%, at least 85%, at least 90%, or at least 95% identical to at least one of SEQ ID NOs: 273, 267, 244, 252, 268, 269, 246, 270, 326, 271, 311, and 437-447.

Embodiment 98b provides the engineered microorganism of any one of Embodiments 68-97, wherein the exogenous polynucleotide encodes a phytase (EC 3.1.3.8, 3.1.3.26, or 3.1.3.72) at least 80%, at least 85%, at least 90%, or at least 95% identical to at least one of SEQ ID NOs: 267, 252, 268, 270, 326, 311, 439, 440, and 446.

Embodiment 98c provides the engineered microorganism of any one of Embodiments 68-97, wherein the exogenous polynucleotide encodes a phytase (EC 3.1.3.8, 3.1.3.26, or 3.1.3.72) at least 80%, at least 85%, at least 90%, or at least 95% identical to at least one of SEQ ID NOs: 267, 269, 268, 270, 326, 439, 440, and 446.

Embodiment 98d provides the engineered microorganism of any one of Embodiments 68-97, wherein the exogenous polynucleotide encodes a phytase (EC 3.1.3.8, 3.1.3.26, or 3.1.3.72) at least 80%, at least 85%, at least 90%, or at least 95% identical to at least one of SEQ ID NOs: 252 and 311.

Embodiment 99 provides the engineered microorganism of any one of Embodiments 68-96, wherein the exogenous polynucleotide encodes an alpha-amylase (EC 3.2.1.1) at least 80%, at least 85%, at least 90%, or at least 95% identical to at least one of SEQ ID NOs: 327-414.

Embodiment 100a provides the engineered microorganism of any one of Embodiments 68-99, wherein the exogenous polynucleotide encodes an alpha-amylase (EC 3.2.1.1) at least 80%, at least 85%, at least 90%, or at least 95% identical to at least one of SEQ ID NOs: 379, 375, 336, 373, 393, 378, 362, 348, 356, and 403.

Embodiment 100b provides the engineered microorganism of any one of Embodiments 68-99, wherein the exogenous polynucleotide encodes an alpha-amylase (EC 3.2.1.1) at least 80%, at least 85%, at least 90%, or at least 95% identical to at least one of SEQ ID NOs: 373, 379, 348, 393, 362, 336, 356, and 403.

Embodiment 100c provides the engineered microorganism of any one of Embodiments 68-99, wherein the exogenous polynucleotide encodes an alpha-amylase (EC 3.2.1.1) at least 80%, at least 85%, at least 90%, or at least 95% identical to at least one of SEQ ID NOs: 375 and 378.

Embodiment 100d provides the engineered microorganism of any one of Embodiments 68-99, wherein the exogenous polynucleotide encodes an alpha-amylase (EC 3.2.1.1) at least 80%, at least 85%, at least 90%, or at least 95% identical to at least one of SEQ ID NOs: 348 and 393.

Embodiment 100e provides the engineered microorganism of any one of Embodiments 68-99, wherein the exogenous polynucleotide encodes an alpha-amylase (EC 3.2.1.1) at least 80%, at least 85%, at least 90%, or at least 95% identical to at least one of SEQ ID NOs: 379 and 373.

Embodiment 101 provides the engineered microorganism of any one of Embodiments 68-100e, wherein at least a portion of the one or more heterologous polypeptides are post-translationally modified and the post-translation modification comprises at least one of acetylation, amidation, hydroxylation, methylation, N-linked glycosylation, O-linked glycosylation, phosphorylation, pyrrolidone carboxylic acid, sulfation, ubiquitylation, or a combination thereof.

Embodiment 102 provides the engineered microorganism of any one of Embodiments 68-101, wherein the medium comprises a carbohydrate.

Embodiment 103 provides the engineered microorganism of Embodiment 102, wherein the carbohydrate comprises a glucose, a glucose oligomer, or a mixture thereof.

Embodiment 104 provides the engineered microorganism of any one of Embodiments 68-103, wherein the one or more heterologous polypeptides are a first class of heterologous polypeptide and the engineered microorganism further comprises a second class of heterologous polypeptide.

Embodiment 105 provides the engineered microorganism of Embodiment 104, wherein the first class of heterologous polypeptide and the second class of heterologous polypeptide are different.

Embodiment 106 provides the engineered microorganism of any one of Embodiments 104 or 105, wherein the second heterologous polypeptide comprises a non-enzymatic heterologous polypeptide.

Embodiment 107 provides the engineered microorganism of Embodiment 106, wherein the non-enzymatic heterologous polypeptide comprises an al-antitrypsin, a hiruidin, a transferrin, an insulin, a serum albumin, collagen, an interferon-alpha 2b, or mixtures thereof.

Embodiment 108 provides the engineered microorganism of any one of Embodiments 68-107, wherein the engineered microorganism further produces a heterologous amino acid, a heterologous antigen, a heterologous cofactor, a heterologous hormone, a heterologous vitamin, a heterologous lipid, a heterologous pharmaceutical, or a mixture thereof.

Embodiment 109 provides the engineered microorganism of Embodiment 108, wherein the amino acid is an essential amino acid to an animal.

Embodiment 110 provides the engineered microorganism of Embodiment 109, wherein the essential amino acid comprises lysine, methionine, phenylalanine, threonine, isoleucine, tryptophan, valine, leucine, arginine, taurine, histidine, or mixtures thereof.

Embodiment 111 provides the engineered microorganism of any one of Embodiments 108-110, wherein the vitamin comprises vitamin A, vitamin B1, vitamin B2, vitamin B3, vitamin B5, vitamin B6, vitamin B7, vitamin B9, vitamin B12, vitamin C, vitamin D1, vitamin D2, vitamin D3, vitamin D4, a tocopherol, vitamin K, or mixtures thereof.

Embodiment 112 provides the engineered microorganism of any one of Embodiments 108-111, wherein the hormone comprises an insulin precursor, a glucagon, or a mixture thereof.

Embodiment 113 provides the engineered microorganism of any one of Embodiments 108-112, wherein the antigen comprises a hepatitis surface antigen.

Embodiment 114 provides the engineered microorganism of any one of Embodiments 68-113, wherein the engineered microorganism secretes comparatively less of the one or more heterologous polypeptides than a corresponding naturally occurring microorganism.

Embodiment 115 provides an engineered yeast adapted to ferment a medium, the engineered yeast comprising:

one or more exogenous polynucleotides encoding one or more heterologous polypeptides that, when expressed from the exogenous polynucleotide are encapsulated in the yeast, the one or more heterologous polypeptides comprising at least about 30% of a total cellular polypeptide level in the yeast, wherein a majority of the one or more heterologous polypeptides exhibit enzymatic activity following a fermentation process to which the yeast is subjected.

Embodiment 116 provides a mixture comprising:

the engineered microorganism of any one of Embodiments 68-115; and

a substrate.

Embodiment 117 provides the mixture of Embodiment 116, wherein the substrate comprises cellulose, a wood chip, a vegetable, a biomass, animal waste, oat, wheat, corn, barley, milo, millet, rice, rye, sorghum, potato, sugar beets, taro, cassaya, a fruit, a fruit juice, a sugar cane, or mixtures thereof.

Embodiment 118 provides the mixture of any one of Embodiments 116 or 117, wherein the substrate comprises animal feed.

Embodiment 119 provides a pharmaceutical comprising:

a heterologous polypeptide isolated from the engineered microorganism of any one of Embodiments 116-118; and

a carrier.

Embodiment 120 provides a composition comprising:

a heterologous polypeptide isolated from the engineered microorganism of any one of Embodiments 116-118; and

a carrier.

Embodiment 121 provides a fermentation method for producing at least about 10 g/L ethanol and one or more heterologous polypeptides, the method comprising:

fermenting a medium using an engineered yeast comprising an expression vector, an edited genome, or a combination thereof, wherein about 20 wt % to about 100 wt % of the one or more heterologous polypeptides are encapsulated intercellularly in the engineered yeast;

introducing an amino acid analogue to the engineered microorganism to result in overexpression of the one or more heterologous polypeptides relative to an engineered microorganism that is free of the amino acid analogue;

isolating the engineered yeast comprising the encapsulated one or more heterologous polypeptides, wherein the one or more heterologous polypeptides exhibit enzymatic activity following isolation of the engineered yeast;

isolating the one or more heterologous polypeptides from the isolated engineered yeast; and

distilling a liquid biproduct produced during fermentation to obtain ethanol.

1. A fermentation method, the method comprising:
contacting a medium with an engineered microorganism comprising an exogenous polynucleotide encoding one or more heterologous polypeptides under fermentation conditions suitable to produce at least 10 g/L of a bioproduct and the one or more heterologous polypeptides, wherein about 2 wt % to about 100 wt % of the one or more heterologous polypeptides are encapsulated intercellularly in the engineered microorganism; and
isolating the engineered microorganism comprising the encapsulated one or more heterologous polypeptides, wherein about 50 wt % to about 100 wt % of the one or more heterologous polypeptides retain functionality following isolation of the engineered microorganism.

2. (canceled)

3. The fermentation method of claim 1, wherein the engineered microorganism comprises Streptococcus, Lactobacillus, Bacillus, Escherichia, Salmonella, Neisseria, Acetobactor, Arthrobacter, Aspergillus, Bifdobacterium, Corynebacterium, Pseudomonas fluorescens, Saccharomyces cerevisiae, Komagataella sp., Kluyveromyces lactis, Yarrowia lipolytica, Issatchenkia orientalis, or a mixture thereof.

4. The fermentation method of claim 1, wherein the engineered microorganism comprises (i) an expression vector comprising or encoding the exogenous polynucleotide; (ii) a genomic modification comprising an insertion of the sequence of the exogenous polynucleotide at a target site; or (iii) a combination thereof.

5. The fermentation method of claim 1, wherein the exogenous polynucleotide is operably linked to a promoter selected from the group consisting of TDH3, GPD1, URAS, TEF1, PDC1, ADH1, PGK1, HXK2, EFB1, SAM2, TPS1, CTT1, HXK5, TPI1, GPD2.

6. (canceled)

7. The fermentation method of claim 1, wherein the bioproduct comprises ethanol, an organic acid, or a combination thereof.

8. The fermentation method of claim 1, wherein at least 25 g/L bioproduct is produced.

9. The fermentation method of claim 1, wherein the encapsulated one or more heterologous polypeptides are at least about 10% of a total cellular polypeptide level in the engineered microorganism.

10. The fermentation method of claim 1, wherein about 70 wt % to about 95 wt % of the of the one or more heterologous polypeptides retain functionality following isolation of the engineered microorganism.

11. The fermentation method of claim 1, wherein the encapsulated one or more heterologous polypeptides comprise a phytase, an alpha-amylase, a glucoamylase, a glucanase, a lipase, an alkaline extracellular protease, an invertase, a galactanase I, an α-amylase, an α-galactosidase, a peroxidase, an aspartic proteinase II, or a mixture thereof.

12. (canceled)

13. The fermentation method of claim 1, wherein the one or more heterologous polypeptides is a phytase and comprises a sequence at least 80%, at least 85%, at least 90%, or at least 95% identical to any one of SEQ ID NOs: 267, 252, 268, 269, 270, 326, 311, 439, 440, and 446.

14. The fermentation method of claim 1, wherein the one or more heterologous polypeptides is an alpha-amylase and comprises a sequence at least 80%, at least 85%, at least 90%, or at least 95% identical to SEQ ID NOs: 379, 375, 336, 373, 393, 378, 362, 348, 356, and 403.

15. The fermentation method of claim 1, wherein the one or more heterologous polypeptides is a pullulanase and comprises a sequence at least 80%, at least 85%, at least 90%, or at least 95% identical to SEQ ID NO:456.

16. The fermentation method of claim 1, further comprising mixing the isolated microorganism with a substrate comprising cellulose, a wood chip, a vegetable, a biomass, animal waste, animal feed, oat, wheat, corn, barley, milo, millet, rice, rye, sorghum, potato, sugar beets, taro, cassaya, a fruit, a fruit juice, a sugar cane, or mixtures thereof.

17. The fermentation method of claim 16, wherein the substrate comprises animal feed.

18. (canceled)

19. An engineered microorganism capable of fermenting a medium to produce at least 25 g/L of a bioproduct, the engineered microorganism comprising:
one or more exogenous polynucleotides encoding one or more heterologous polypeptides that, when expressed, are encapsulated in the engineered microorganism, the one or more heterologous polypeptides comprising at least about 5% of a total cellular polypeptide level in the engineered microorganism, wherein about 50 wt % to about 100 wt % of the one or more heterologous polypeptides retain their functionality following isolation of the engineered microorganism.

20. The engineered microorganism of claim 19, wherein the engineered microorganism comprises Streptococcus, Lactobacillus, Bacillus, Escherichia, Salmonella, Neisseria, Acetobactor, Arthrobacter, Aspergillus, Bifdobacterium, Corynebacterium, Pseudomonas fluorescens, Saccharomyces cerevisiae, Komagataella sp., Kluyveromyces lactis, Yarrowia lipolytica, Issatchenkia orientalis, or a mixture thereof.

21. The engineered microorganism of claim 19, wherein the engineered microorganism comprises (i) an expression vector comprising or encoding the exogenous polynucleotide; (ii) a genomic modification comprising an insertion of the sequence of the exogenous polynucleotide at a target site; or (iii) a combination thereof.

22. The engineered microorganism of claim 19, wherein the bioproduct is ethanol.

23.-26. (canceled)

27. The engineered microorganism of claim 19, wherein the one or more heterologous polypeptides is a phytase and comprises a sequence at least 80% identical to any one of SEQ ID NOs: 267, 252, 268, 269, 270, 326, 311, 439, 440, and 446.

28. The engineered microorganism of claim 19, wherein the one or more heterologous polypeptides is an alpha-amylase and comprises a sequence at least 80% identical to SEQ ID NOs: 379, 375, 336, 373, 393, 378, 362, 348, 356, and 403.

29. The engineered microorganism of claim 19, wherein the one or more heterologous polypeptides is a pullulanase and comprises a sequence at least 80% identical to SEQ ID NO: 456.

30. A mixture comprising:
the engineered microorganism of claim 19; and
a substrate comprising cellulose, a wood chip, a vegetable, a biomass, animal waste, animal feed, oat, wheat, corn, barley, milo, millet, rice, rye, sorghum, potato, sugar beets, taro, cassava, a fruit, a fruit juice, a sugar cane, or mixtures thereof.

31. (canceled)

32. The mixture of claim 30, wherein the substrate is an animal feed.

33. The fermentation method of claim 1, wherein the bioproduct is ethanol and the method produces at least about 10 g/L ethanol and one or more heterologous polypeptides.

34. The method of claim 33, wherein the one or more heterologous polypeptides retains 0.1 U/mg to 5 U/mg activity following isolation of the yeast.

35.-38. (canceled)

39. The method of claim 33, wherein the exogenous polynucleotide encodes a phytase at least 80% identical to at least one of SEQ ID NOs: 273, 267, 244, 252, 268, 269, 246, 270, 326, 271, 311, and 437-447.

40. The method of claim 33, wherein the exogenous polynucleotide encodes a phytase at least 80% identical to at least one of SEQ ID NOs: 267, 252, 268, 270, 326, 311, 439, 440, and 446.

41. The method of claim 33, wherein the exogenous polynucleotide encodes an alpha-amylase (EC 3.2.1.1) at least 80% identical to at least one of SEQ ID NOs: 379, 375, 336, 373, 393, 378, 362, 348, 356, and 403.

42. The method of claim 33, wherein the method produces at least 50 g/L ethanol.
